(19) United States
(12) Patent Application Publication
(10) Pub. No.: US 2011/0039732 A1
(43) Pub. Date: Feb. 17, 2011

(54) cDNA Synthesis Using Non-Random Primers

(75) Inventors: Christopher Raymond, Seattle, WA (US); Christopher Armour, Kirkland, WA (US); John Castle, Mainz (DE)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/767,542

(22) Filed: Apr. 26, 2010

Related U.S. Application Data
(63) Continuation of application No. PCT/US2008/081206, filed on Oct. 24, 2008.
(60) Provisional application No. 60/983,085, filed on Oct. 26, 2007.

(51) Int. Cl. C40B 40/08; C12P 19/34; C40B 50/06; C12Q 1/68; C12N 9/10; C12N 9/00; C12N 9/22

(52) U.S. Cl. 506/16; 435/91.2; 506/26; 435/6; 435/193; 435/183; 435/199

(57) ABSTRACT

The present invention provides methods for selectively amplifying a target population of nucleic acid molecules in a population of RNA template molecules (e.g., all mRNA molecules expressed in a cell type except for the most highly expressed mRNA species). The present invention also provides a first population of oligonucleotides including the nucleic acid sequences set forth in SEQ ID NOS:1-749 and a second population of oligonucleotides including the nucleic acid sequences set forth in SEQ ID NOS:750-1498. The first population of oligonucleotides can be used, for example, to prime the synthesis of first strand cDNA molecules complementary to mRNA molecules isolated from mammalian cells without priming the synthesis of cDNA molecules complementary to ribosomal RNA molecules. The second population of oligonucleotides can be used, for example, to prime the second strand synthesis of primer extension products (first strand cDNA) complementary to mRNA molecules isolated from mammalian cells without priming the second strand synthesis of primer extension products synthesized from ribosomal RNA molecules.

### CROSS-REFERENCE(S) TO RELATED APPLICATION(S)

This application is a continuation of PCT/US2008/081206, filed on Oct. 24, 2008, which application claims the benefit of U.S. Provisional Application No. 60/983,085, filed on Oct. 26, 2007. Said applications are incorporated by reference herein in their entirety.

### FIELD OF THE INVENTION

The present invention relates to methods of selectively amplifying target nucleic acid molecules and oligonucleotides useful for priming the amplification of target nucleic acid molecules.

### BACKGROUND

Gene expression analysis often involves amplification of starting nucleic acid molecules. Amplification of nucleic acid molecules may be accomplished by reverse transcription (RT), in vitro transcription (IVT) or the polymerase chain reaction (PCR), either individually or in combination. The starting nucleic acid molecules may be mRNA molecules, which are amplified by first synthesizing complementary cDNA molecules, then synthesizing second cDNA molecules that are complementary to the first cDNA molecules, thereby producing double stranded cDNA molecules. The synthesis of first strand cDNA is typically accomplished using a reverse transcriptase and the synthesis of second strand cDNA is typically accomplished using a DNA polymerase. The double stranded cDNA molecules may be used to make complementary RNA molecules using an RNA polymerase, resulting in amplification of the original starting mRNA molecules. The RNA polymerase requires a promoter sequence to direct initiation of RNA synthesis. Complementary RNA molecules may, for example, be used as a template to make additional complementary DNA molecules. Alternatively, the double stranded cDNA molecules may be amplified, for example, by PCR and the amplified PCR products may be used as sequencing templates or in microarray analysis.

Amplification of nucleic acid molecules requires the use of oligonucleotide primers that specifically hybridize to one or more target nucleic acid molecules in the starting material. Each oligonucleotide primer may include a promoter sequence that is located 5′ to the hybridizing portion of the oligonucleotide that hybridizes to the target nucleic acid molecule(s). If the hybridizing portion of an oligonucleotide is too short, then the oligonucleotide does not stably hybridize to a target nucleic acid molecule and priming and subsequent amplification does not occur. Also, if the hybridizing portion of an oligonucleotide is too short, then the oligonucleotide does not specifically hybridize to one or a small number of target nucleic acid molecules, but nonspecifically hybridizes to numerous target nucleic acid molecules.

Amplification of a complex mixture of different target nucleic acid molecules (e.g., RNA molecules) typically requires the use of a population of numerous oligonucleotides having different nucleic acid sequences. The cost of the oligonucleotides increases with the length of the oligonucleotides. In order to control costs, it is preferable to make oligonucleotide primers that are no longer than the minimum length required to ensure specific hybridization of an oligonucleotide to a target sequence.

It is often undesirable to amplify highly expressed RNAs (e.g., ribosomal RNAs). For example, in gene expression experiments that analyze expression of genes in blood cells, amplification of numerous copies of abundant globin mRNAs, or ribosomal RNAs, may obscure subtle changes in the levels of rare mRNAs. Thus, there is a need for populations of oligonucleotide primers that selectively amplify desired nucleic acid molecules within a population of nucleic acid molecules (e.g., oligonucleotide primers that selectively amplify all mRNAs that are expressed in a cell except for the most highly expressed RNAs). In order to reduce the cost of synthesizing the population of oligonucleotides, the hybridizing portion of each oligonucleotide should be no longer than necessary to ensure specific hybridization to a desired target sequence under defined conditions.

### SUMMARY

In one aspect, the present invention provides methods for selectively amplifying a target population of nucleic acid molecules within a larger non-target population of nucleic acid molecules (e.g., all RNA molecules expressed in a cell type except for the most highly expressed RNA species). The methods of this aspect of the invention each include the steps of (a) providing a population of single-stranded primer extension products synthesized from a population of RNA template molecules in a sample isolated from a mammalian subject using reverse transcriptase enzyme and a first population of oligonucleotide primers, wherein each oligonucleotide in the first population of oligonucleotide primers comprises a hybridizing portion and a defined sequence portion located 5′ to the hybridizing portion, wherein the population of RNA template molecules comprises a target population of nucleic acid molecules and a non-target population of nucleic acid molecules; (b) synthesizing double-stranded cDNA from the population of single-stranded primer extension products according to step (a) using a DNA polymerase and a second population of oligonucleotide primers, wherein each oligonucleotide in the second population of oligonucleotides comprises a hybridizing portion, wherein the hybridizing portion consists of one of 6, 7, or 8 nucleotides and a defined sequence located 5′ to the hybridizing portion wherein the hybridizing portion is selected from all possible oligonucleotides having a length of 6, 7, or 8 nucleotides that do not hybridize under the defined conditions to the non-target population of nucleic acid molecules in the synthesized single-stranded cDNA. In some embodiments, each oligonucleotide in the first population of oligonucleotide comprises a random hybridizing portion and a defined sequence located 5′ to the hybridizing portion.

In another aspect, the present invention provides methods of selectively amplifying a target population of nucleic acid molecules within a larger non-target population of nucleic acid molecules. The methods of this aspect of the invention comprise the steps of (a) synthesizing single-stranded cDNA from a sample comprising total RNA isolated from a mammalian subject using reverse transcriptase enzyme and a first population of oligonucleotide primers, wherein each oligonucleotide within the first population of oligonucleotide primers comprises a hybridizing portion and a defined sequence portion located 5′ to the hybridizing portion, wherein the hybridizing portion is a member of the population of oligonucleotides comprising SEQ ID NOS:1-749; and (b) synthesizing double-stranded cDNA from the single-stranded cDNA synthesized according to step (a) using a DNA polymerase and a second population of oligonucleotide primers, wherein each oligonucleotide within the second population of oligonucleotide primers comprises a hybridizing portion and a defined sequence portion located 5′ to the hybridizing portion, wherein the hybridizing portion is a member of the population of oligonucleotides comprising SEQ ID NOS:750-1498.

In another aspect, the present invention provides methods for transcriptome profiling. The methods of this aspect of the invention comprise (a) synthesizing a population of single stranded primer extension products from a target population of nucleic acid molecules within a population of RNA template molecules in a sample isolated from a subject using reverse transcriptase enzyme and a first population of oligonucleotide primers comprising a hybridizing portion and a first PCR primer binding site located 5′ to the hybridizing portion; (b) synthesizing double-stranded cDNA from the population of single-stranded primer extension products generated according to step (a) using a DNA polymerase and a second population of oligonucleotide primers comprising a hybridizing portion and a second PCR primer binding site located 5′ to the hybridizing portion; and (c) PCR amplifying the double-stranded cDNA generated according to step (b) using a first PCR primer that binds to the first PCR primer binding site and a second PCR primer that binds to the second PCR primer binding site, wherein the non-target population of nucleic acid molecules consists essentially of ribosomal RNA and mitochondrial ribosomal RNA of the same species as the mammalian subject.

In another aspect, the present invention provides populations of oligonucleotides comprising SEQ ID NOS:1-749. These oligonucleotides can be used, for example, to prime the synthesis of first-strand cDNA molecules complementary to RNA molecules isolated from a mammalian subject without priming the synthesis of first strand cDNA molecules complementary to ribosomal RNA (18S, 28S) or mitochondrial ribosomal RNA (12S, 16S) molecules. In some embodiments, each oligonucleotide in the population of oligonucleotides further comprises a defined sequence portion located 5′ to the hybridizing portion. In one embodiment, the defined sequence portion comprises a transcriptional promoter, which may be used as a primer binding site in PCR amplification, or for in vitro transcription. In another embodiment, the defined sequence portion comprises a primer binding site that is not a transcriptional promoter. For example, in some embodiments the present invention provides populations of oligonucleotides wherein a transcriptional promoter, such as the T7 promoter (SEQ ID NO:1508), is located 5′ to a member of the population of oligonucleotides having the sequences set forth in SEQ ID NOS:1-749. Thus, in some embodiments, the present invention provides populations of oligonucleotides wherein each oligonucleotide consists of the T7 promoter (SEQ ID NO:1508) located 5′ to a different member of the population of oligonucleotides having the sequences set forth in SEQ ID NOS:1-749. In further embodiments, the present invention provides populations of oligonucleotides wherein the defined sequence portion comprises at least one primer binding site that is useful for priming a PCR synthesis reaction and that does not include an RNA polymerase promoter sequence. A representative example of a defined sequence portion for use in such embodiments is provided as 5′TCCGATCTCT3′ (SEQ ID NO:1499), which is preferably located 5′ to a member of the population of oligonucleotides having the sequences set forth in SEQ ID NOS:1-749.

In another aspect, the present invention provides populations of oligonucleotides comprising SEQ ID NOS:750-1498. These oligonucleotides can be used, for example, to prime the synthesis of second strand cDNA molecules complementary to first strand cDNA molecules synthesized from RNA isolated from a mammalian subject without priming the synthesis of second strand cDNA molecules complementary to first strand cDNA reverse transcribed from ribosomal RNA (18S, 28S) or mitochondrial ribosomal RNA (12S, 16S) molecules. In some embodiments, each oligonucleotide in the population of oligonucleotides further comprises a defined sequence portion located 5′ to the hybridizing portion. In one embodiment, the defined sequence portion comprises a transcriptional promoter, which may be used as a primer binding site in PCR amplification or for in vitro transcription. In another embodiment, the defined sequence portion comprises a primer binding site that is not a transcriptional promoter. For example, in some embodiments, the present invention provides populations of oligonucleotides wherein a transcriptional promoter, such as the T7 promoter (SEQ ID NO:1508), is located 5′ to a member of the population of oligonucleotides having the sequences set forth in SEQ ID NOS:750-1498. Thus, in some embodiments, the present invention provides populations of oligonucleotides wherein each oligonucleotide consists of the T7 promoter (SEQ ID NO:1508) located 5′ to a different member of the population of oligonucleotides having the sequences set forth in SEQ ID NOS:750-1498. In further embodiments, the present invention provides populations of oligonucleotides wherein the defined sequence portion comprises at least one primer binding site that is useful for priming a PCR synthesis reaction and that does not include an RNA polymerase promoter sequence. A representative example of a defined sequence portion for use in such embodiments is provided as 5′TCCGATCTGA3′ (SEQ ID NO:1500), which is preferably located 5′ to a member of the population of oligonucleotides having the sequences set forth in SEQ ID NOS:750-1498.

In another aspect, the present invention provides a reagent for selectively amplifying a target population of nucleic acid molecules in a larger population of non-target nucleic acid molecules. In one embodiment, the reagent comprises at least 10% of the oligonucleotides comprising SEQ ID NOS:1-749. In another embodiment, the reagent comprises at least 10% of the oligonucleotides comprising SEQ ID NOS:750-1498.

In another aspect, the present invention provides a kit for selectively amplifying a target population of nucleic acid molecules. The kit of this aspect of the invention comprises a reagent comprising a first population of oligonucleotides for first strand cDNA synthesis, wherein each oligonucleotide in the first population of oligonucleotides comprises a hybridizing portion and a defined sequence portion located 5′ to the hybridizing portion, wherein the hybridizing portion is a member of the population of oligonucleotides comprising SEQ ID NOS:1-749. In some embodiments, the kit further comprises a second population of oligonucleotides for second strand cDNA synthesis, wherein each oligonucleotide in the second population of oligonucleotides comprises a hybridizing portion and a defined sequence portion located 5′ to the hybridizing portion, wherein the hybridizing portion is a member of the population of oligonucleotides comprising SEQ ID NOS:750-1498.

In another aspect, the present invention provides a population of selectively amplified nucleic acid molecules comprising a representation of a transcriptome of a mammalian subject comprising a 5′ defined sequence, a population of amplified sequences corresponding to a nucleic acid expressed in the mammalian subject, a 3′ defined sequence wherein the population of amplified sequences is characterized by having the following properties with reference to the particular mammalian species: (a) having greater than 75% polyadenylated and non-polyadenylated transcripts and having less than 10% ribosomal RNA.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A shows the number of exact matches for random 6-mers (N6) oligonucleotides on nucleotide sequences in the human RefSeq transcript database as described in Example 1;

FIG. 1B shows the number of exact matches for Not-So-Random (NSR) 6-mer oligonucleotides on nucleotide sequences in the human RefSeq transcript database as described in Example 1;

FIG. 1C shows a representative embodiment of the methods of the invention for synthesizing a preparation of selectively amplified cDNA molecules using a mixture of random primers for first strand cDNA synthesis and a mixture of anti-NSR 6-mer oligonucleotides for second strand cDNA synthesis, as described in Example 2;

FIG. 1D shows a representative embodiment of the methods of the invention for synthesizing a preparation of selectively amplified aDNA molecules using a mixture of NSR 6-mer oligonucleotides for first strand cDNA synthesis and a mixture of anti-NSR6-mer oligonucleotides for second strand cDNA synthesis, followed by PCR amplification, as described in Example 2 and Example 4;

FIG. 2 is flow diagram illustrating a method of whole transcriptome analysis of a subject comprising selectively amplifying nucleic acid molecules from RNA isolated from the subject followed by sequence analysis or microarray analysis of the amplified nucleic acid molecules as described in Example 4 and Example 5;

FIG. 3A is a histogram plot on a logarithmic scale showing the relative abundance of 18S, 28S, 12Sn and 16S (normalized to gene and N8) in a population of first strand cDNA molecules synthesized using various NSR-6 pools as compared to first strand cDNA generated using random primers (N8=100%) as described in Example 3;

FIG. 3B graphically illustrates the relative levels of abundance of cytoplasmic rRNA (18S or 28S) in cDNA amplified using random primers (N7) in both first strand and second strand synthesis (N7>N7=100% 18S, 100% 28S) as compared to cDNA amplified using NSR primers (SEQ ID NOS:1-749) in the first strand followed by random primers (N7) in the second strand (NSR>N7=3.0% 18S, 3.4% 28S), and as compared to cDNA amplified using NSR primers (SEQ ID NOS:1-749) in the first strand followed by anti-NSR primers (SEQ ID NOS:750-1498) in the second strand (NSR>anti-NSR=0.1% 18S, 0.5% 28S) as described in Example 3;

FIG. 3C graphically illustrates the relative levels of abundance of mitochondrial rRNA (12S or 16S) in cDNA amplified using random primers (N7) in both first strand and second strand synthesis (N7>N7=100% 12S, or 16S) as compared to cDNA amplified using NSR primers (SEQ ID NOS:1-749) in the first strand followed by random primers (N7) in the second strand (NSR>N7=27% 12S, 20.4% 16S), and as compared to cDNA amplified using NSR primers (SEQ ID NOS:1-749) in the first strand followed by anti-NSR primers (SEQ ID NOS:750-1498) in the second strand (NSR>anti-NSR=8.2% 12S, 3.5% 16S) as described in Example 3;

FIG. 4A is a histogram plot showing the gene-specific polyA content of representative gene transcripts in cDNA synthesized using various NSR primers during first strand synthesis as described in Example 3;

FIG. 4B is a histogram plot showing the relative abundance level of representative non-polyadenylated RNA transcripts in cDNA amplified from Jurkat-1 and Jurkat-2 total RNA using various NSR primers during first strand cDNA synthesis as described in Example 3;

FIG. 5 graphically illustrates the log ratio of Jurkat/K562 mRNA expression data measured in cDNA generated using NSR-6mers (x-axis) versus the log ratio of Jurkat/K562 mRNA expression data measured in cDNA generated using random primers (N8), as described in Example 3;

FIG. 6A graphically illustrates the proportion of rRNA to mRNA in total RNA typically obtained after polyA purification, demonstrating that even after 95% removal of rRNA from total RNA, the remaining RNA consists of a mixture of about 50% rRNA and 50% mRNA as described in Example 3;

FIG. 6B graphically illustrates the proportion of rRNA to mRNA in a cDNA sample prepared using NSR primers during first strand cDNA synthesis and anti-NSR primers during second strand cDNA synthesis. As shown, in contrast to polyA purification, the use of NSR primers and anti-NSR primers to generate cDNA from total RNA is effective to remove 99.9% rRNA, resulting in a cDNA population enriched for greater than 95% mRNA as described in Example 3;

FIG. 7A graphically illustrates the detection and positional distribution of polyA+ RefSeq mRNA in NSR-primed (dotted line) or expressed sequence tag (EST) (solid line) cDNAs across long transcripts (≧4 kb), illustrating the combined read frequencies for 5,790 transcripts shown at each base position starting from the 5′ termini, as described in Example 7;

FIG. 7B graphically illustrates the detection and positional distribution of polyA+ RefSeq mRNA in NSR-primed (dotted line) or expressed sequence tag (EST) (solid line) cDNAs across long transcripts (≧4 kb), illustrating the combined read frequencies for 5,790 transcripts shown at each base position starting from the 3′ termini, as described in Example 7; and

FIG. 8 graphically illustrates the enrichment of small nucleolar RNAs (snoRNAs) encoded by the Chromosome 15 Prader-Willi neurological disease locus in NSR-primed cDNA generated from RNA isolated from whole brain relative to NSR-primed cDNA generated from RNA isolated from the Universal Human Reference (UHR) cell line, as described in Example 7.

### DETAILED DESCRIPTION

Unless specifically defined herein, all terms used herein have the same meaning as they would to one skilled in the art of the present invention. Practitioners are particularly directed to Sambrook et al., Molecular Cloning: A Laboratory Manual, 2d ed., Cold Spring Harbor Press, Plainsview, N.Y.; and Ausubel et al., Current Protocols in Molecular Biology (Supplement 47), John Wiley & Sons, New York, 1999, for definitions and terms of the art.

The use of Not-So-Random (“NSR”) 6-mer primers for first strand cDNA synthesis is described in co-pending U.S. patent application Ser. No. 11/589,322, filed Oct. 27, 2006, incorporated herein by reference. In a particular embodiment, the NSR-timers described in co-pending U.S. patent application Ser. No. 11/589,322 comprise populations of oligonucleotides that hybridize to all mRNA molecules expressed in blood cells but that do not hybridize to globin mRNA (HBA1, HBA2, HBB, HBD, HBG1 and HBG2) or to nuclear ribosomal RNA (18S and 28S rRNA). In the present application, a different population of NSR primers (SEQ ID NOS:1-749) is provided that includes oligonucleotides that hybridize to all mRNA molecules expressed in mammalian cells, including globin mRNA, but that do not hybridize to nuclear ribosomal RNA (18S and 28S rRNA) and mitochondrial ribosomal RNAs (12S and 16S mt-rRNA). The present application further provides a second population of anti-NSR oligonucleotides (SEQ ID NOS:750-1498) for use during second strand cDNA synthesis. The anti-NSR oligonucleotides (SEQ ID NOS:750-1498) are selected to hybridize to all first strand cDNA molecules reverse transcribed from RNA templates expressed in mammalian cells, including globin mRNA, but that do not hybridize to first strand cDNA molecules transcribed from nuclear ribosomal RNA (18S and 28S rRNA) and mitochondrial ribosomal RNAs (12S and 16S mt-rRNA). As described in Examples 1-4, the use of a first round of selective amplification using NSR primers (SEQ ID NOS:1-749) during first strand synthesis followed by a second round of selective amplification using anti-NSR primers (SEQ ID NOS:750-1498) during second strand synthesis results in a population of double stranded cDNA that represents substantially all of the polyA RNA and non-polyA RNA expressed in the cell, with a very low level (less than 10%) of nucleic acid molecules representing unwanted nuclear ribosomal RNA and mitochondrial ribosomal RNA. As shown in FIG. 2, the invention also provides methods which analyze the products of the amplification methods of the invention, such as sequencing and gene expression profiling (e.g., microarray analysis).

In accordance with the foregoing, in one aspect, the present invention provides methods for selectively amplifying a target population of nucleic acid molecules within a larger non-target population of nucleic acid molecules (e.g., all RNA molecules expressed in a cell type except for the most highly expressed RNA species). The methods of this aspect of the invention each include the steps of (a) synthesizing single-stranded cDNA from RNA in a sample isolated from a mammalian subject using reverse transcriptase enzyme and a first population of oligonucleotide primers, wherein each oligonucleotide in the first population of oligonucleotide primers comprises a hybridizing portion and a defined sequence portion located 5′ to the hybridizing portion, wherein the RNA comprises a target population of nucleic acid molecules within a larger non-target population of nucleic acid molecules; and (b) synthesizing double-stranded cDNA from the single-stranded cDNA synthesized according to step (a) using a DNA polymerase and a second population of oligonucleotide primers, wherein each oligonucleotide in the second population of oligonucleotides comprises a hybridizing portion, wherein the hybridizing portion consists of one of 6, 7, or 8 nucleotides and a defined sequence located 5′ to the hybridizing portion wherein the hybridizing portion is selected from all possible oligonucleotides having a length of 6, 7, or 8 nucleotides that do not hybridize under the defined conditions to the non-target population of nucleic acid molecules in the synthesized single-stranded cDNA.

The second population of oligonucleotides may also include a defined sequence portion located 5′ to the hybridizing portion. In one embodiment, the defined sequence portion comprises a transcriptional promoter that can also be used as a primer binding site. Therefore, in certain embodiments of this aspect of the invention, each oligonucleotide of the second population of oligonucleotides comprises a hybridizing portion that consists of 6 nucleotides or 7 nucleotides or 8 nucleotides and a transcriptional promoter portion located 5′ to the hybridizing portion. In another embodiment, the defined sequence portion of the second population of oligonucleotides includes a second primer binding site for use in a PCR amplification reaction and that may optionally include a transcriptional promoter. By way of example, the populations of anti-NSR oligonucleotides provided by the present invention are useful in the practice of the methods of this aspect of the invention.

For example, in one embodiment of the present invention, a population of oligonucleotides (SEQ ID NOS:750-1498), that each has a length of 6 nucleotides, was identified that can be used as primers to prime the second strand synthesis of all, or substantially all, first strand cDNA molecules synthesized from a target population of RNA molecules from mammalian cells but that do not prime the second strand synthesis of first strand cDNA reverse transcribed from non-target ribosomal RNA (rRNA) or mitochondrial rRNA (mt-rRNA) from mammalian cells. The identified second population of oligonucleotides (SEQ ID NOS:750-1498) is referred to as anti-Not-So-Random (anti-NSR) primers. Thus, this population of oligonucleotides (SEQ ID NOS:750-1498) can be used to prime the second strand synthesis of a population of first strand nucleic acid molecules (e.g., cDNAs) that are representative of a starting population of mRNA molecules isolated from mammalian cells but do not prime second strand synthesis of cDNA molecules that correspond to rRNA or mt-rRNAs.

In other embodiments, each oligonucleotide in the first population of oligonucleotides comprises a hybridizing portion, wherein the hybridizing portion consists of one of 6, 7, or 8 nucleotides and a defined sequence located 5′ to the hybridizing portion wherein the hybridizing portion is selected from all possible oligonucleotides having a length of 6, 7, or 8 nucleotides that do not hybridize under the defined conditions to the non-target population of nucleic acid molecules in a sample comprising RNA from a mammalian subject.

The first population of oligonucleotides may also include a defined sequence portion located 5′ to the hybridizing portion. In one embodiment, the defined sequence portion comprises a transcriptional promoter that can also be used as a first primer binding site. Therefore, in certain embodiments of this aspect of the invention, each oligonucleotide of the first population of oligonucleotides comprises a hybridizing portion that consists of 6 nucleotides or 7 nucleotides or 8 nucleotides and a transcriptional promoter portion located 5′ to the hybridizing portion. In another embodiment, the defined sequence portion of the first population of oligonucleotides includes a first primer binding site for use in a PCR amplification reaction and that may optionally include a transcriptional promoter. By way of example, the populations of NSR oligonucleotides provided by the present invention are useful in the practice of the methods of this aspect of the invention.

For example, in one embodiment of the present invention, a first population of oligonucleotides (SEQ ID NOS:1-749) wherein each has a length of 6 nucleotides, was identified that can be used as primers to prime the first strand synthesis of all, or substantially all, mRNA molecules from mammalian cells, but that do not prime the amplification of non-target ribosomal RNA (rRNA) or mitochondrial rRNA (mt-rRNA) from mammalian cells. The identified first population of oligonucleotides (SEQ ID NOS:1-749) is referred to as Not-So-Random (NSR) primers. Thus, this population of oligonucleotides (SEQ ID NOS:1-749) can be used to prime the first strand synthesis of a population of nucleic acid molecules (e.g., cDNAs) that are representative of a starting population of mRNA molecules isolated from mammalian cells but do not prime first strand synthesis of cDNA molecules that correspond to rRNA or mt-rRNAs.

The present invention also provides a first population of oligonucleotides for priming first strand cDNA synthesis, wherein a defined sequence, such as the T7 promoter (SEQ ID NO:1508) or a first primer binding site (SEQ ID NO:1499) is located 5′ to a member of the population of oligonucleotides having the sequences set forth in SEQ ID NOS:1-749. Thus, each oligonucleotide may include a hybridizing portion (selected from SEQ ID NOS:1-749) that hybridizes to target nucleic acid molecules (e.g., mRNAs) and a defined sequence, such as a promoter sequence or first primer binding site, is located 5′ to the hybridizing portion. The defined sequence portion may be incorporated into DNA molecules amplified using the oligonucleotides (that include the T7 promoter) as primers and can thereafter promote transcription from the DNA molecules.

Alternatively, the defined sequence portion, such as the transcriptional promoter or first primer binding site, may be covalently attached to the cDNA molecule, for example, by DNA ligase enzyme.

Useful transcription promoter sequences include the T7 promoter (5′AATTAATACGACTCACTATAGGGAGA3′ (SEQ ID NO:1508)), the SP6 promoter (5′ATTTAGGTGACACTATAGAAGNG3′ (SEQ ID NO:1509)), and the T3 promoter (5′AATTAACCCTCACTAAAGGGAGA3′ (SEQ ID NO:1510)).

The target nucleic acid population can include, for example, all mRNAs expressed in a cell or tissue except for a selected group of non-target mRNAs such as, for example, the most abundantly expressed mRNAs. A non-target abundantly expressed mRNA typically constitutes at least 0.1% of all the mRNA expressed in the cell or tissue (and may constitute, for example, more than 50% or more than 60% or more than 70% of all the mRNA expressed in the cell or tissue). An example of an abundantly expressed non-target mRNA is ribosomal rRNA or mitochondrial rRNA in mammalian cells. Other examples of abundantly expressed non-target RNA that one could selectively eliminate using the methods of the invention include, for example, globin mRNA (from blood cells) or chloroplast rRNA (from plant cells).

The methods of the invention are useful for transcriptome profiling of total RNA in a biological cell sample in which it is desirable to reduce the presence of a group of RNAs (that do not hybridize to the NSR and/or anti-NSR primers) from an amplified sample, such as, for example, highly expressed RNAs (e.g., ribosomal RNAs). In some embodiments, the methods of the invention may be used to reduce the amount of a group of nucleic acid molecules that do not hybridize to the NSR primers and/or anti-NSR primers in amplified nucleic acid derived from an RNA sample by at least 2 fold up to 1000 fold, such as at least 10 fold, 50 fold, 100 fold, 500 fold or greater, in comparison to the amount of amplified nucleic acid molecules that do hybridize to the NSR and/or anti-NSR primers.

Populations of oligonucleotides used to practice the method of this aspect of the invention are selected from within a larger population of oligonucleotides, wherein the first population of oligonucleotides is selected based on its ability to hybridize under defined conditions to a target RNA population but not hybridize under the defined conditions to a non-target RNA population and the first population of oligonucleotides comprises all possible oligonucleotides having a length of 6 nucleotides, 7 nucleotides, or 8 nucleotides.

The second population of oligonucleotides is selected based on its ability to hybridize under defined conditions to a target first strand cDNA population, but not hybridize under the defined conditions to a non-target first strand cDNA population and the second population of oligonucleotides comprises all possible oligonucleotides having a length of 6 nucleotides, 7 nucleotides, or 8 nucleotides. In one embodiment, the second population of oligonucleotides may be generated by synthesizing the reverse complement of the sequence of the first population of oligonucleotides.

Composition of First Population of Oligonucleotides. In some embodiments, the first population of oligonucleotides includes all possible oligonucleotides having a length of 6 nucleotides or 7 nucleotides or 8 nucleotides. The first population of oligonucleotides may include only all possible oligonucleotides having a length of 6 nucleotides or all possible oligonucleotides having a length of 7 nucleotides or all possible oligonucleotides having a length of 8 nucleotides. Optionally, the first population of oligonucleotides may include other oligonucleotides in addition to all possible oligonucleotides having a length of 6 nucleotides or all possible oligonucleotides having a length of 7 nucleotides or all possible oligonucleotides having a length of 8 nucleotides. Typically, each member of the first population of oligonucleotides is no more than 30 nucleotides long.

Sequences of First Population of Oligonucleotides. There are 4,096 possible oligonucleotides having a length of 6 nucleotides, 16,384 possible oligonucleotides having a length of 7 nucleotides, and 65,536 possible oligonucleotides having a length of 8 nucleotides. The sequences of the oligonucleotides that constitute the population of oligonucleotides can readily be generated by a computer program such as Microsoft Word®.

Selection of Subpopulation of First Oligonucleotides. The subpopulation of first oligonucleotides is selected from the population of oligonucleotides based on the ability of the members of the subpopulation of first oligonucleotides to hybridize under defined conditions to a population of target nucleic acids but not hybridize under the same defined conditions to a non-target population. A sample of amplified includes target nucleic acid molecules (e.g., RNA or DNA molecules) that are to be amplified (e.g., using reverse transcription) and also includes non-target nucleic acid molecules that are not to be amplified. The subpopulation of first oligonucleotides is made up of oligonucleotides that each hybridize under defined conditions to target sequences distributed throughout the population of the nucleic acid molecules that are to be amplified but that do not hybridize under the same defined conditions to most (or any) of the non-target nucleic acid molecules that are not to be amplified. The subpopulation of first oligonucleotides hybridizes under defined conditions to target nucleic acid sequences other than those that have been intentionally avoided (non-target sequences).

For example, the cell sample may include a population of all mRNA molecules expressed in mammalian cells including many ribosomal RNA molecules (e.g., 5S, 18S, and 28S ribosomal RNAs) and mitochondrial rRNA molecules (e.g., 12S and 16S ribosomal RNAs). It is typically undesirable to amplify the ribosomal RNAs. For example, in gene expression experiments that analyze expression of genes in cells, amplification of numerous copies of abundant ribosomal RNAs may obscure subtle changes in the levels of less abundant mRNAs. Consequently, in the practice of the present invention, a subpopulation of first oligonucleotides is selected that does not hybridize under defined conditions to most (or any) non-target ribosomal RNAs but that does hybridize under the same defined conditions to most (preferably all) of the other target mRNA molecules expressed in the cells.

In order to select a subpopulation of first oligonucleotides that hybridizes under defined conditions to a target nucleic acid population but does not hybridize under the defined conditions to a non-target nucleic acid population, it is necessary to know the complete or substantially complete nucleic acid sequences of the member(s) of the non-target nucleic acid population. Thus, for example, it is necessary to know the nucleic acid sequences of the 5S, 18S, and 28S ribosomal RNAs (or a representative member of each of the foregoing classes of ribosomal RNA) and the nucleic acid sequences of the 12S and 16S ribosomal mitochondrial RNAs. The sequences for the ribosomal RNAs for the mammalian species from which the cell sample is obtained can be found in a publically accessible database. For example, the NCBI Genbank identifiers are provided in TABLE 1 for human 12S, 16S, 18S, and 28S ribosomal RNA, as accessed on Sep. 5, 2007.

A suitable software program is then used to compare the sequences of all of the oligonucleotides in the population of first oligonucleotides (e.g., the population of all possible 6 nucleic acid oligonucleotides) to the sequences of the ribosomal RNAs to determine which of the oligonucleotides will hybridize to any portion of the ribosomal RNAs under defined hybridization conditions. Only the oligonucleotides that do not hybridize to any portion of the ribosomal RNAs under defined hybridization conditions are selected. Perl script may easily be written that permits comparison of nucleic acid sequences and identification of sequences that hybridize to each other under defined hybridization conditions.

Thus, for example, as described more fully in Example 1, the subpopulation of all possible 6 nucleic acid oligonucleotides that were not exactly complementary to any portion of any ribosomal RNA sequence was identified. In general, the subpopulation of oligonucleotides (that hybridizes under defined conditions to a target nucleic acid population but does not hybridize under the defined conditions to a non-target nucleic acid population) must contain enough different oligonucleotide sequences to hybridize to all or substantially all nucleic acid molecules in the RNA sample. Example 1 herein shows that the population of oligonucleotides having the nucleic acid sequences set forth in SEQ ID NOS:1-749 hybridizes to all or substantially all nucleic acid sequences within a population of gene transcripts stored in the publicly accessible database called RefSeq.

Additional Defined Nucleic Acid Sequence Portions. The selected subpopulation of first oligonucleotides (e.g., SEQ ID NOS:1-749) can be used to prime the reverse transcription of a target population of RNA molecules to generate first strand cDNA. Alternatively, a population of first oligonucleotides can be used as primers wherein each oligonucleotide includes the sequence of one member of the selected subpopulation of oligonucleotides and also includes an additional defined nucleic acid sequence. The additional defined nucleic acid sequence is typically located 5′ to the sequence of the member of the selected subpopulation of oligonucleotides. Typically, the population of oligonucleotides includes the sequences of all members of the selected subpopulation of oligonucleotides (e.g., the population of oligonucleotides can include all of the sequences set forth in SEQ ID NOS:1-749).

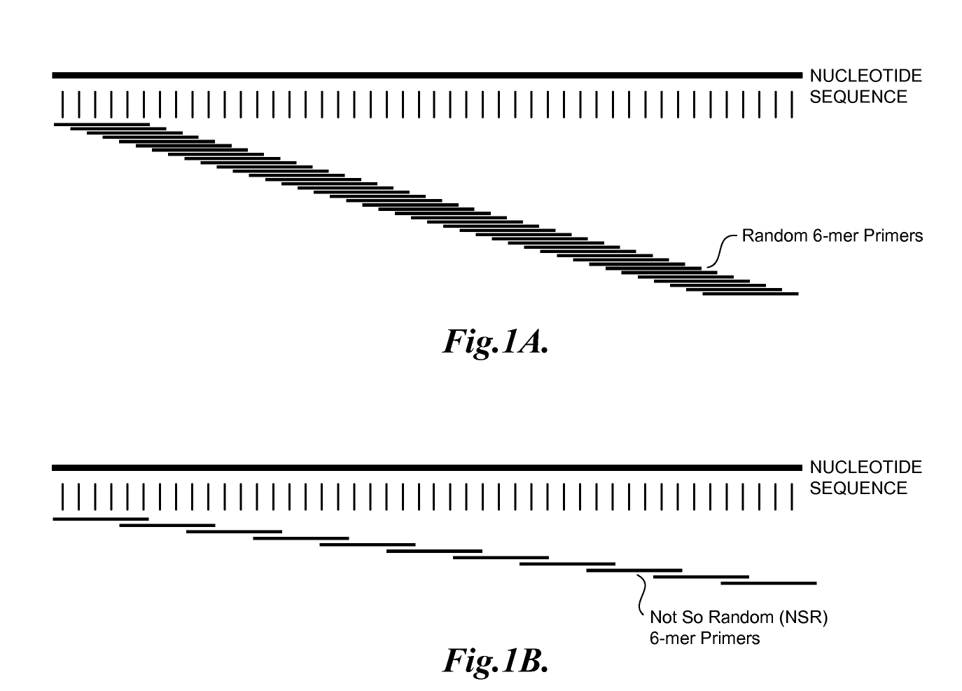

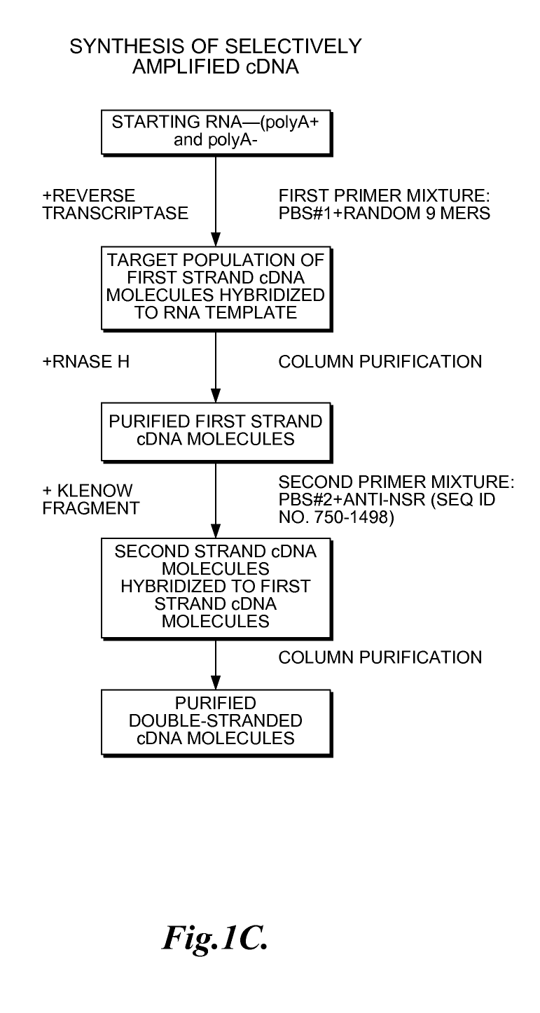

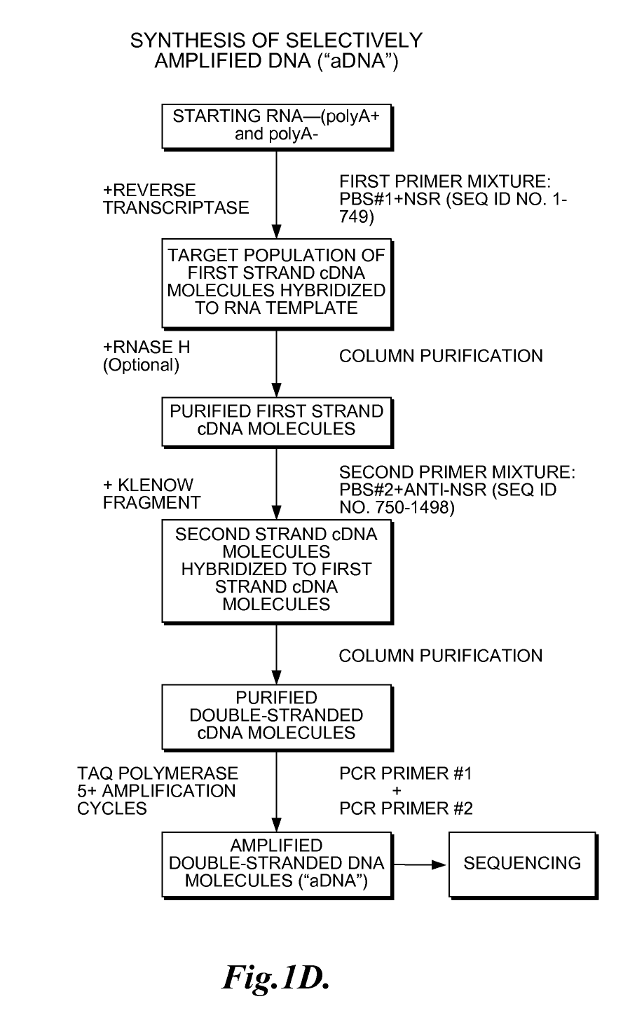

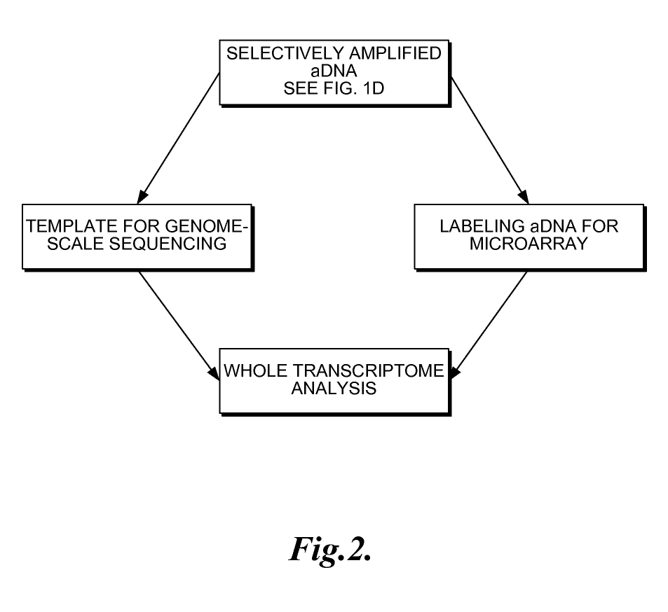

The additional defined nucleic acid sequence is selected so that it does not affect the hybridization specificity of the oligonucleotide to a complementary target sequence. For example, as shown in FIG. 1D, each first oligonucleotide can include a transcriptional promoter sequence or first primer binding site (PBS#1) located 5′ to the sequence of the member of the selected subpopulation of oligonucleotides. The promoter sequence may be incorporated into the amplified nucleic acid molecules which can, therefore, be used as templates for the synthesis of RNA. Any RNA polymerase promoter sequence can be included in the defined sequence portion of the population of oligonucleotides. Representative examples include the T7 promoter (SEQ ID NO:1508), the SP6 promoter (SEQ ID NO:1509), and the T3 promoter (SEQ ID NO:1510).

In some embodiments of this aspect of the invention, as shown in FIG. 1C, each oligonucleotide in the first population of oligonucleotides comprises a random hybridizing portion and a defined sequence located 5′ to the hybridizing portion. As shown in FIG. 1C, each first oligonucleotide can include a defined sequence comprising a primer binding site located 5′ to the random hybridizing portion. The primer binding site is incorporated into the amplified nucleic acids, which can then be used as a PCR primer binding site for the generation of double-stranded amplified DNA products from the cDNA. The primer binding site may be a portion of a transcriptional promoter sequence.

Sequences of Second Population of Oligonucleotides. The selection process for the second population of oligonucleotides is similar to the process described above for the selection of the first population of oligonucleotides with the difference being that the hybridizing portion consisting of 6 nucleotides, 7 nucleotides, or 8 nucleotides is selected to hybridize to the first strand cDNA reverse transcribed from the target RNA under defined conditions and not hybridize to the first strand cDNA reverse transcribed from the non-target RNA under defined conditions. The second population of oligonucleotides can be selected using the methods described above, for example, using the publicly available sequences for ribosomal RNA. The second population of oligonucleotides can also be generated as the reverse-complement of the first population of oligonucleotides (anti-NSR).

Thus, for example, as described more fully in Example 1, the second population was selected based on all possible 6 nucleic acid oligonucleotides that were not exactly complementary to any portion of any ribosomal RNA sequence was identified. Example 1 herein shows that the population of oligonucleotides having the nucleic acid sequences set forth in SEQ ID NOS:1-749 hybridizes to all or substantially all nucleic acid sequences within a population of gene transcripts stored in the publicly accessible database called RefSeq. A second population SEQ ID NOS:750-1498 (anti-NSR) was then generated that was the reverse complement of the first population of oligonucleotides (SEQ ID NOS:1-749, NSR).

Additional Defined Nucleic Acid Sequence Portions. The selected subpopulation of second oligonucleotides (e.g., SEQ ID NOS:750-1498) can be used to prime the second strand cDNA synthesis of a target population of first strand cDNA molecules. Alternatively, a population of second oligonucleotides can be used as primers wherein each oligonucleotide includes the sequence of one member of the selected subpopulation of oligonucleotides and also includes an additional defined nucleic acid sequence. The additional defined nucleic acid sequence is typically located 5′ to the sequence of the member of the selected subpopulation of oligonucleotides. Typically, the population of oligonucleotides includes the sequences of all members of the selected subpopulation of oligonucleotides (e.g., the population of oligonucleotides can include all of the sequences set forth in SEQ ID NOS:750-1498).

The additional defined nucleic acid sequence is selected so that it does not affect the hybridization specificity of the oligonucleotide to a complementary target sequence. For example, as shown in FIG. 1D, each first oligonucleotide can include a transcriptional promoter sequence or second primer binding site (PBS#2) located 5′ to the sequence of the member of the selected subpopulation of oligonucleotides. The promoter sequence may be incorporated into the amplified nucleic acid molecules that can, therefore, be used as templates for the synthesis of RNA. Any RNA polymerase promoter sequence can be included in the defined sequence portion of the population of oligonucleotides. Representative examples include the T7 promoter (SEQ ID NO:1508), the SP6 promoter (SEQ ID NO:1509), and the T3 promoter (SEQ ID NO:1510).

In another aspect, the present invention provides a population of first oligonucleotides wherein each oligonucleotide of the population includes (a) a sequence of a 6 nucleic acid oligonucleotide that is a member of a subpopulation of oligonucleotides (SEQ ID NOS:1-749), wherein the subpopulation of oligonucleotides hybridizes to all or substantially all RNAs expressed in mammalian cells but does not hybridize to ribosomal RNAs; and (b) a primer binding site (PBS#1) sequence (SEQ ID NO:1499) located 5′ to the sequence of the 6 nucleic acid oligonucleotide. In one embodiment, the population of first oligonucleotides includes all of the 6 nucleotide sequences set forth in SEQ ID NOS:1-749. In another embodiment, the population of first oligonucleotides includes at least 10% (such as at least 20%, 30%, 40%, 50%, 60%, 70%, 80%, 85%, 90%, 95%, or 99%) of the 6 nucleotide sequences set forth in SEQ ID NOS:1-749.

Optionally, a spacer portion is located between the defined sequence portion and the hybridizing portion in the first population of oligonucleotides. The spacer portion is typically from 1 to 12 nucleotides long (e.g., from 1 to 6 nucleotides long) and can include any combination of random nucleotides (N=A, C, T, or G). The spacer portion can, for example, be composed of a random selection of nucleotides. All or part of the spacer portion may or may not hybridize to the same target nucleic acid sequence as the hybridizing portion. If all or part of the spacer portion hybridizes to the same target nucleic acid sequence as the hybridizing portion, then the effect is to enhance the efficiency of cDNA synthesis primed by the oligonucleotide that includes the hybridizing portion and the hybridizing spacer portion. In some embodiments, the population of first oligonucleotides further comprises a spacer region consisting of from 1 to 10 random nucleotides (A, C, T, or G) located between the primer binding site and the hybridizing portion. In another embodiment, the population of first oligonucleotides includes all of the six nucleotide sequences set forth in SEQ ID NOS:1-749 wherein each nucleotide sequence further comprises at least one spacer nucleotide at the 5′ end.

In another aspect, the present invention provides a population of second oligonucleotides wherein each oligonucleotide of the population includes (a) a sequence of a 6 nucleic acid oligonucleotide that is a member of a subpopulation of oligonucleotides (SEQ ID NOS:750-1498), wherein the subpopulation of oligonucleotides hybridizes to all or substantially all first strand cDNAs reverse transcribed from RNAs expressed in mammalian cells but does not hybridize to first strand cDNAs reverse transcribed from ribosomal RNAs; and (b) a primer binding site (PBS#2) sequence (SEQ ID NO:1500) located 5′ to the sequence of the 6 nucleic acid oligonucleotide. In one embodiment, the population of first oligonucleotides includes all of the 6 nucleotide sequences set forth in SEQ ID NOS:750-1498. In another embodiment, the population of first oligonucleotides includes at least 10% (such as at least 20%, 30%, 40%, 50%, 60%, 70%, 80%, 85%, 90%, 95%, or 99%) of the 6 nucleotide sequences set forth in SEQ ID NOS:750-1498.

Optionally, a spacer portion is located between the defined sequence portion and the hybridizing portion in the second population of oligonucleotides. The spacer portion is typically from 1 to 12 nucleotides long (e.g., from 1 to 6 nucleotides long) and can include any combination of random nucleotides (N=A, C, T, or G). The spacer portion can, for example, be composed of a random selection of nucleotides. All or part of the spacer portion may or may not hybridize to the same target nucleic acid sequence as the hybridizing portion. If all or part of the spacer portion hybridizes to the same target nucleic acid sequence as the hybridizing portion, then the effect is to enhance the efficiency of cDNA synthesis primed by the oligonucleotide that includes the hybridizing portion and the hybridizing spacer portion. In some embodiments, the population of first oligonucleotides further comprises a spacer region consisting of from 1 to 10 random nucleotides (A, C, T, or G) located between the primer binding site and the hybridizing portion. In another embodiment, the population of first oligonucleotides includes all of the six nucleotide sequences set forth in SEQ ID NOS:750-1498, wherein each nucleotide sequence further comprises at least one spacer nucleotide at the 5′ end.

In some embodiments, the defined sequence portion of the first population of oligonucleotides and the defined sequence portion of the second population of oligonucleotides each consists of a length ranging from at least 10 nucleotides up to 30 nucleotides, such as from 10 to 12 nucleotides, from 10 to 14 nucleotides, from 10 to 16 nucleotides, from 10 to 18 nucleotides, and from 10 to 20 nucleotides. In some embodiments, the defined sequence portion of each of the first and second population of oligonucleotides consists of 10 nucleotides, wherein the defined sequence portion comprises a PCR primer binding site, and wherein at least 8 consecutive nucleotides in the PCR binding site in each member of the first population of oligonucleotides have an identical sequence with at least 8 nucleotides in the PCR binding site in each member of the second population of oligonucleotides. In a further embodiment, the defined sequence portion of each of the first and second population of oligonucleotides consists of 10 nucleotides, wherein the defined sequence portion comprises a PCR primer binding site, and wherein at least 8 consecutive nucleotides in the PCR binding site in each member of the first population of oligonucleotides have an identical sequence with at least 8 nucleotides in the PCR binding site in each member of the second population of oligonucleotides, and wherein the remaining two nucleotides at the 3′ end of the defined sequence portion in the first population of oligonucleotides are different (e.g., C, T) from the two nucleotides at the 3′ end of the defined sequence portion in the second population of oligonucleotides (e.g., G, A), thereby allowing for the identification of the transcript strand (sense or antisense) after sequence analysis prior to alignment of the sequence reads.

In a further embodiment, hybrid RNA/DNA oligonucleotides are provided wherein the defined sequence portion of the first population of oligonucleotides comprises an RNA portion and a DNA portion, wherein the RNA portion is 5′ with respect to the DNA portion. In one embodiment, the 5′ RNA portion of the hybrid primer consists of at least 11 RNA nucleotide defined sequence portions and the 3′ DNA portion of the hybrid primer consists of at least three DNA nucleotides. In a specific embodiment, the hybrid RNA/DNA oligonucleotides comprise SEQ ID NO:1558 covalently attached to the 5′ end of the NSR primers (SEQ ID NOS:1-749). The cDNA generated using the hybrid RNA/DNA oligonucleotides may be used as a template for generating single-stranded amplified DNA using the methods described in U.S. Pat. No. 6,946,251, hereby incorporated by reference, as further described in Example 6.

For example, a first population of oligonucleotides for first strand cDNA synthesis comprising a hybrid RNA/DNA defined sequence portion (SEQ ID NO:1558) and a hybridizing portion (SEQ ID NOS:1-749) forms the basis for replication of the target nucleic acid molecules in template RNA. The first population of oligonucleotides comprising the hybrid RNA/DNA primer portion hybridize to the target RNA in the RNA templates and the hybrid RNA/DNA primer is extended by an RNA-dependent DNA polymerase to form a first primer extension product (first strand cDNA). After cleavage of the template RNA, a second strand cDNA is formed in a complex with the first primer extension product. In accordance with this embodiment, the double-stranded complex of first and second primer extension products is composed of an RNA/DNA hybrid at one end due to the presence of the hybrid primer in the first primer extension product. The double-stranded complex is then used to generate single-stranded DNA amplification products with an agent such as an enzyme which cleaves RNA from the RNA/DNA hybrid (such as RNAseH) which cleaves the RNA sequence from the hybrid, leaving a sequence on the second primer extension product available for binding by another hybrid primer, which may or may not be the same as the first hybrid primer. Another first primer extension product is produced by a highly processive DNA polymerase, such as phi29, which displaces the previously bound cleaved first primer extension product, resulting in displaced cleaved first primer extension product.

In an alternative embodiment, a double-stranded complex for single-stranded DNA amplification is generated by modifying a double-stranded cDNA product (all DNA), generated using either random primers or NSR and anti-NSR primers, or a combination thereof. The double-stranded cDNA product is denatured and an RNA/DNA hybrid primer is annealed to a pre-determined primer sequence at the 3′ end portion of the second strand cDNA. The DNA portion of the hybrid primer is then extended using reverse transcriptase to form a double-stranded complex with an RNA hybrid portion. The double-stranded complex is then used as a template for single-stranded DNA amplification by first treating with RNAseH to remove the RNA portion of the complex, adding the RNA/DNA hybrid primer, and adding a highly processive DNA polymerase, such as phi29 to generate single-stranded DNA amplification products.

Hybridization Conditions. In the practice of the present invention, a population of first oligonucleotides is selected from a population of oligonucleotides based on the ability of the members of the population of oligonucleotides to hybridize under defined conditions to a target nucleic acid population but not hybridize under the same defined conditions to a non-target nucleic acid population. The defined hybridization conditions permit the first oligonucleotides to specifically hybridize to all nucleic acid molecules that are present in the sample except for ribosomal RNAs. Typically, hybridization conditions are no more than 25° C. to 30° C. (for example, 10° C.) below the melting temperature (Tm) of the native duplex. Tm for nucleic acid molecules greater than about 100 bases can be calculated by the formula Tm=81.5+0.41% (G+C)−log(Na+), wherein (G+C) is the guanosine and cytosine content of the nucleic acid molecule. For oligonucleotide molecules less than 100 bases in length, exemplary hybridization conditions are 5° C. to 10° C. below Tm. On average, the Tm of a short oligonucleotide duplex is reduced by approximately (500/oligonucleotide length)° C. In some embodiments of the present invention, the hybridization temperature is in the range of from 40° C. to 50° C. The appropriate hybridization conditions may also be identified empirically without undue experimentation.

In one embodiment of the present invention, the first population of oligonucleotides hybridizes to a target population of nucleic acid molecules at a temperature of about 40° C.

In one embodiment of the present invention, the second population of oligonucleotides hybridizes to a target population of nucleic acid molecules in a population of single-stranded primer extension products at a temperature of about 37° C.

Amplification Conditions. In the practice of the present invention, the amplification of the first subpopulation of a target nucleic acid population occurs under defined amplification conditions. Hybridization conditions can be chosen as described, supra. Typically, the defined amplification conditions include first strand cDNA synthesis using a reverse transcriptase enzyme. The reverse transcription reaction is performed in the presence of defined concentrations of deoxynucleotide triphosphates (dNTPs). In some embodiments, the dNTP concentration is in a range from about 1000 to about 2000 microMolar in order to enrich the amplified product for target genes, as described in co-pending U.S. patent application Ser. No. 11/589,322, filed Oct. 27, 2006, incorporated herein by reference.

Composition and Synthesis of Oligonucleotides. An oligonucleotide primer useful in the practice of the present invention can be DNA, RNA, PNA, chimeric mixtures, or derivatives or modified versions thereof, as long as it is still capable of priming the desired reaction. The oligonucleotide primer can be modified at the base moiety, sugar moiety, or phosphate backbone and may include other appending groups or labels, so long as it is still capable of priming the desired amplification reaction.

For example, an oligonucleotide primer may comprise at least one modified base moiety that is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxymethyluracil, 5-methoxyuracil, 2-methylthio-N-6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, and 2,6-diaminopurine.

Again by way of example, an oligonucleotide primer can include at least one modified sugar moiety selected from the group including, but not limited to, arabinose, 2-fluoroarabinose, xylulose, and hexose.

By way of further example, an oligonucleotide primer can include at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

An oligonucleotide primer for use in the methods of the present invention may be derived by cleavage of a larger nucleic acid fragment using non-specific nucleic acid cleaving chemicals or enzymes, or site-specific restriction endonucleases, or by synthesis by standard methods known in the art, for example, by use of an automated DNA synthesizer (such as are commercially available from Biosearch, Applied Biosystems, etc.) and standard phosphoramidite chemistry. As examples, phosphorothioate oligonucleotides may be synthesized by the method of Stein et al. (Nucl. Acids Res. 16:3209-3221, 1988) and methylphosphonate oligonucleotides can be prepared by use of controlled pore glass polymer supports (Sarin et al., Proc. Natl. Acad. Sci. U.S.A. 85:7448-7451, 1988).

Once the desired oligonucleotide is synthesized, it is cleaved from the solid support on which it was synthesized and treated by methods known in the art to remove any protecting groups present. The oligonucleotide may then be purified by any method known in the art, including extraction and gel purification. The concentration and purity of the oligonucleotide may be determined by examining an oligonucleotide that has been separated on an acrylamide gel or by measuring the optical density at 260 nm in a spectrophotometer.

The methods of this aspect of the invention can be used, for example, to selectively amplify coding regions of mRNAs, introns, alternatively spliced forms of a gene, and non-coding RNAs that regulate gene expression.

In another aspect, the present invention provides populations of oligonucleotides comprising at least 10% (such as at least 20%, 30%, 40%, 50%, 60%, 70%, 80%, 85%, 90%, 95%, or 99%) of the nucleic acid sequences set forth in SEQ ID NOS:1-749. These oligonucleotides (SEQ ID NOS:1-749) can be used, for example, to prime the first strand synthesis of cDNA molecules complementary to RNA molecules isolated from a mammalian subject without priming the first strand synthesis of cDNA molecules complementary to ribosomal RNA molecules. Indeed, these oligonucleotides (SEQ ID NOS:1-749) can be used, for example, to prime the synthesis of cDNA using any population of RNA molecules as templates, without amplifying a significant amount of ribosomal RNAs or mitochondrial ribosomal RNAs. For example, the present invention provides populations of oligonucleotides wherein a defined sequence portion, such as a transcriptional promoter such as the T7 promoter (SEQ ID NO:1508), or a primer binding site (PBS#1) (SEQ ID NO:1499) is located 5′ to a member of the population of oligonucleotides having the sequences set forth in SEQ ID NOS:1-749. Thus, in some embodiments, the present invention provides populations of oligonucleotides wherein each oligonucleotide consists of the T7 promoter (SEQ ID NO:1508) located 5′ to a different member of the population of oligonucleotides having the sequences set forth in SEQ ID NOS:1-749. In some embodiments, the present invention provides populations of oligonucleotides wherein each oligonucleotide consists of the primer binding site SEQ ID NO:1499 and a random spacer nucleotide (A, C, T, or G) is located 5′ to a different member of the population of oligonucleotides having the sequences set forth in SEQ ID NOS:1-749. In some embodiments, the population of oligonucleotides includes at least 10% (such as 20%, 30%, 40%, 50%, 60%, 70%, 80%, 90%, 95%, or 99%) of the six nucleotide sequences set forth in SEQ ID NOS:1-749.

In another aspect, the present invention provides populations of oligonucleotides comprising at least 10% (such as at least 20%, 30%, 40%, 50%, 60%, 70%, 80%, 85%, 90%, 95%, or 99%) of the nucleic acid sequences set forth in SEQ ID NOS:750-1498. These oligonucleotides (SEQ ID NOS:750-1498) can be used, for example, to prime the second strand synthesis of single-stranded primer extension products complementary to RNA molecules isolated from a mammalian subject without priming the second strand synthesis of cDNA molecules complementary to ribosomal RNA molecules. Indeed, these oligonucleotides (SEQ ID NOS:750-1498) can be used, for example, to prime the synthesis second strand cDNA using any population of single stranded primer extension molecules as templates, without amplifying a significant amount of single-stranded primer extension molecules that are complementary to ribosomal RNAs or mitochondrial ribosomal RNAs. For example, the present invention provides populations of oligonucleotides wherein a defined sequence portion, such as a transcriptional promoter such as the T7 promoter (SEQ ID NO:1508), or a primer binding site (PBS#2) (SEQ ID NO:1500) is located 5′ to a member of the population of oligonucleotides having the sequences set forth in SEQ ID NOS:750-1498. Thus, in some embodiments, the present invention provides populations of oligonucleotides wherein each oligonucleotide consists of the T7 promoter (SEQ ID NO:1508) located 5′ to a different member of the population of oligonucleotides having the sequences set forth in SEQ ID NOS:750-1498. In some embodiments, the present invention provides populations of oligonucleotides wherein each oligonucleotide consists of the primer binding site (PBS#2) SEQ ID NO:1500 and a random spacer nucleotide (A, C, T, or G) is located 5′ to a different member of the population of oligonucleotides having the sequences set forth in SEQ ID NOS:750-1498. In some embodiments, the population of oligonucleotides includes at least 10% (such as 20%, 30%, 40%, 50%, 60%, 70%, 80%, 90%, 95%, or 99%) of the six nucleotide sequences set forth in SEQ ID NOS:750-1498.

In another aspect, the present invention provides a reagent for selectively synthesizing single-stranded primer extension products (first strand cDNA) from a population of RNA template molecules. The reagent can be used, for example, to prime the synthesis of first strand cDNA molecules complementary to target RNA template molecules in a sample isolated from a mammalian subject without priming the synthesis of first strand cDNA molecules complementary to ribosomal RNA molecules. The reagent of the present invention comprises a population of oligonucleotides comprising at least 10% of the nucleic acid sequences set forth in SEQ ID NOS:1-749. In some embodiments, the present invention provides a reagent comprising a population of oligonucleotides that includes at least 10% (such as 20%, 30%, 40%, 50%, 60%, 70%, 80%, 85%, 90%, 95%, or 99%) of the six nucleotide sequences set forth in SEQ ID NOS:1-749. In some embodiments, the population of oligonucleotides is selected to hybridize to substantially all nucleic acid molecules that are present in a sample except for ribosomal RNAs and mitochondrial rRNAs. In other embodiments, the population of oligonucleotides is selected to hybridize to a subset of nucleic acid molecules that are present in a sample, wherein the subset of nucleic acid molecules does not include ribosomal RNAs.

In another aspect, the present invention provides a reagent for selectively synthesizing double-stranded cDNA from a population of single-stranded primer extension products (first strand cDNA). The reagent can be used, for example, to prime the synthesis of second strand cDNA molecules that are complementary to target RNA template molecules in a sample isolated from a mammalian subject without priming the synthesis of second-strand cDNA molecules complementary to ribosomal RNA molecules. The reagent in accordance with this aspect of the invention may be used to prime the synthesis of first strand cDNA generated using random primers, or may be used to prime the synthesis of first strand cDNA generated using NSR primers, such as SEQ ID NO:1-749, in order to provide an additional step of selectivity of target molecules. The reagent according to this aspect of the present invention comprises a population of oligonucleotides comprising at least 10% of the nucleic acid sequences set forth in SEQ ID NOS:750-1498. In some embodiments, the present invention provides a reagent comprising a population of oligonucleotides that includes at least 10% (such as 20%, 30%, 40%, 50%, 60%, 70%, 80%, 85%, 90%, 95%, or 99%) of the six nucleotide sequences set forth in SEQ ID NOS:750-1498. In some embodiments, the population of oligonucleotides is selected to hybridize to substantially all first strand cDNA molecules that are present in a sample except for first strand cDNA synthesized from ribosomal RNAs and mitochondrial rRNAs. In other embodiments, the population of oligonucleotides is selected to hybridize to a subset of first strand cDNA molecules that are present in a sample, wherein the subset of first strand cDNA molecules does not include cDNA molecules synthesized from ribosomal RNAs.

In another embodiment, the present invention provides a reagent that comprises a population of oligonucleotides wherein a defined sequence portion comprising a transcriptional promoter such as the T7 promoter is located 5′ to a member of the population of oligonucleotides having the sequences set forth in SEQ ID NOS:1-749. Thus in some embodiments, the present invention provides a reagent comprising populations of oligonucleotides wherein each oligonucleotide consists of the T7 promoter (SEQ ID NO:1508) located 5′ to a different member of the population of oligonucleotides having the sequences set forth in SEQ ID NOS:1-749. In another embodiment, the present invention provides a reagent that comprises a population of oligonucleotides wherein a defined sequence portion comprising a primer binding site (e.g., PBS#1) is located 5′ to a member of the population of oligonucleotides having the sequences set forth in SEQ ID NOS:1-749. Thus, in some embodiments, the present invention provides a reagent comprising populations of oligonucleotides wherein each oligonucleotide consists of the primer binding site (PBS#1) (SEQ ID NO:1499) located 5′ to a different member of the population of oligonucleotides having the sequences set forth as SEQ ID NOS:1-749. In some embodiments, the present invention provides a reagent the further comprises a spacer region of at least one random nucleotide located between the primer binding site and a different member of the population of oligonucleotides having the sequences set forth as SEQ ID NOS:1-749.

In another embodiment, the present invention provides a reagent that comprises a population of oligonucleotides wherein a defined sequence portion comprising a transcriptional promoter such as the T7 promoter is located 5′ to a member of the population of oligonucleotides having the sequences set forth in SEQ ID NOS:750-1498. Thus in some embodiments, the present invention provides a reagent comprising populations of oligonucleotides wherein each oligonucleotide consists of the T7 promoter (SEQ ID NO:1508) located 5′ to a different member of the population of oligonucleotides having the sequences set forth in SEQ ID NOS:750-1498. In another embodiment, the present invention provides a reagent that comprises a population of oligonucleotides wherein a defined sequence portion comprising a primer binding site (e.g., PBS#2) is located 5′ to a member of the population of oligonucleotides having the sequences set forth in SEQ ID NOS:750-1498. Thus in some embodiments, the present invention provides a reagent comprising populations of oligonucleotides wherein each oligonucleotide consists of the primer binding site (PBS#2) (SEQ ID NO:1500) located 5′ to a different member of the population of oligonucleotides having the sequences set forth as SEQ ID NOS:750-1498. In some embodiments, the present invention provides a reagent the further comprises a spacer region of at least one random nucleotide located between the primer binding site and a different member of the population of oligonucleotides having the sequences set forth as SEQ ID NOS:750-1498.

The reagents of the present invention can be provided as an aqueous solution or an aqueous solution with the water removed or a lyophilized solid.

In a further embodiment, the reagent of the present invention may include one or more of the following components for the production of double-stranded cDNA: a reverse transcriptase, a DNA polymerase, a DNA ligase, an RNase H enzyme, a Tris buffer, a potassium salt, a magnesium salt, an ammonium salt, a reducing agent, deoxynucleoside triphosphates (dNTPs), [beta]-nicotinamide adenine dinucleotide (β-NAD+), and a ribonuclease inhibitor. For example, the reagent may include components optimized for first strand cDNA synthesis, such as a reverse transcriptase with reduced RNase H activity and increased thermal stability (e.g., SuperScript™ III Reverse Transcriptase, Invitrogen), and a final concentration of dNTPs in the range of from 50 to 5000 microMolar or, more preferably, in the range of from 1000 to 2000 microMolar.

In another aspect, the present invention provides kits for selectively amplifying a target population of nucleic acid molecules within a population of RNA template molecules in a sample obtained from a mammalian subject. In some embodiments, the kits comprise (a) a first reagent that comprises a first population of oligonucleotide primers wherein a defined sequence portion such as a primer binding site (PBS#1) is located 5′ to a hybridizing portion consisting of 6 nucleotides selected from all possible oligonucleotides having a length of 6 nucleotides that do not hybridize under defined conditions to the non-target population of nucleic acid molecules in the population of RNA template molecules, wherein the non-target population of nucleic acid molecules consists essentially of the most abundant nucleic acid molecules in the population of RNA template molecules, (b) a second reagent that comprises a second population of oligonucleotide primers wherein a defined sequence portion such as a primer binding site (PBS#2), is located 5′ to a hybridizing portion consisting of 6 nucleotides selected from the reverse complement of the nucleotide sequence of the hybridizing portions of the first population of oligonucleotide primers, and (c) a first PCR primer that binds to the first defined sequence portion of the first population of oligonucleotides and a second PCR primer that binds to the second defined sequence portion of the second population of oligonucleotides.

In some embodiments, the first reagent comprises a member of the population of oligonucleotides having the sequences set forth in SEQ ID NOS:1-749. Thus in some embodiments, the present invention provides kits containing a first reagent comprising a first population of oligonucleotides wherein each oligonucleotide consists of a first primer binding site (PBS#1) (SEQ ID NO:1499) located 5′ to a different member of the population of oligonucleotides having the sequences set forth in SEQ ID NOS:1-749. In some embodiments, the present invention provides kits containing a second reagent comprising a second population of oligonucleotides wherein each oligonucleotide consists of a second primer binding site (PBS#2) (SEQ ID NO:1500) located 5′ to a different member of the population of oligonucleotides having the sequences set forth in SEQ ID NOS:750-1498. In some embodiments, the invention provides kits containing a first PCR primer comprising at least 10 consecutive nucleotides that hybridize to the defined sequence portion in the first oligonucleotide population, and optionally comprises an additional sequence tail that does not hybridize to the first oligonucleotide population and a second PCR primer comprising at least 10 consecutive nucleotides that hybridize to the defined sequence portion in the second oligonucleotide population, and optionally comprises an additional sequence tail that does not hybridize to the second oligonucleotide population. In one embodiment, the first PCR primer consists of SEQ ID NO:1501, and the second PCR primer consists of SEQ ID NO:1502. The kits according to this embodiment are useful for producing amplified PCR products from cDNA generated using the Not-So-Random primers (SEQ ID NOS:1-749) and the anti-NSR (SEQ ID NOS:750-1498) primers of the invention.

The kits of the invention may be designed to detect any target nucleic acid population, for example, all RNAs expressed in a cell or tissue except for the most abundantly expressed RNAs, in accordance with the methods described herein. Nonlimiting examples of exemplary oligonucleotide primers include SEQ ID NOS:1-749. Nonlimiting examples of primer binding regions are set forth as SEQ ID NOS:1499 and 1500.

The spacer portion may include any combination of nucleotides including nucleotides that hybridize to the target RNA.

In certain embodiments, the kit comprises a reagent comprising oligonucleotide primers with hybridizing portions of 6, 7, or 8 nucleotides.

In certain embodiments, the kit comprises a reagent comprising a population of oligonucleotide primers that may be used to detect a plurality of mammalian mRNA targets.

In certain embodiments, the kit comprises oligonucleotides that hybridize in the temperature range of from 40° C. to 50° C.

In another embodiment, the kit comprises a subpopulation of oligonucleotides that do not detect rRNA or mitochondrial rRNA. Exemplary oligonucleotides for use in accordance with this embodiment of the kit are provided in SEQ ID NOS:1-749 and SEQ ID NOS:750-1498.

In some embodiments, the kits comprises a reagent comprising a population of oligonucleotides comprising at least 10% (such as at least 20%, 30%, 40%, 50%, 60%, 70%, 80%, 85%, 90%, 95%, or 99%) of the six nucleotide sequences set forth in SEQ ID NOS:1-749.

In some embodiments, the kits comprise a reagent comprising a population of oligonucleotides comprising at least 10% (such as at least 20%, 30%, 40%, 50%, 60%, 70%, 80%, 85%, 90%, 95%, or 99%) of the six nucleotide sequences set forth in SEQ ID NOS:750-1498.

In certain embodiments, the kit includes oligonucleotides wherein the transcription promoter comprises the T7 promoter (SEQ ID NO:1508), the SP6 promoter (SEQ ID NO:1509), or the T3 promoter (SEQ ID NO:1510).

In another embodiment, the kit may comprise oligonucleotides with a spacer portion of from 1 to 12 nucleotides that comprises any combination of nucleotides.

In some embodiments of the present invention, the kit may further comprise one or more of the following components for the production of cDNA: a reverse transcriptase enzyme a DNA polymerase enzyme, a DNA ligase enzyme, an RNase H enzyme, a Tris buffer, a potassium salt (e.g., potassium chloride), a magnesium salt (e.g., magnesium chloride), an ammonium salt (e.g., ammonium sulfate), a reducing agent (e.g., dithiothreitol), deoxynucleoside triphosphates (dNTPs), [beta]-nicotinamide adenine dinucleotide (β-NAD+), and a ribonuclease inhibitor. For example, the kit may include components optimized for first strand cDNA synthesis, such as a reverse transcriptase with reduced RNase H activity and increased thermal stability (e.g., SuperScript™ III Reverse Transcriptase, Invitrogen), and a dNTP stock solution to provide a final concentration of dNTPs in the range of from 50 to 5000 microMolar or, more preferably, in the range of from 1000 to 2000 microMolar.

In various embodiments, the kit may include a detection reagent such as SYBR green dye or BEBO dye that preferentially or exclusively binds to double-stranded DNA during a PCR amplification step. In other embodiments, the kit may include a forward and/or reverse primer that includes a fluorophore and quencher to measure the amount of the PCR amplification products.

A kit of the invention can also provide reagents for in vitro transcription of the amplified cDNAs. For example, in some embodiments the kit may further include one or more of the following components: a RNA polymerase enzyme, an IPPase (Inositol polyphosphate 1-phosphatase) enzyme, a transcription buffer, a Tris buffer, a sodium salt (e.g., sodium chloride), a magnesium salt (e.g., magnesium chloride), spermidine, a reducing agent (e.g., dithiothreitol), nucleoside triphosphates (ATP, CTP, GTP, UTP), and amino-allyl-UTP.

In another embodiment, the kit may include reagents for labeling the in vitro transcription products with Cy3 or Cy5 dye for use in hybridizing the labeled cDNA samples to microarrays.

In another embodiment, the kit may include reagents for labeling the double-stranded PCR products. For example, the kit may include reagents for incorporating a modified base, such as amino-allyl dUTP, during PCR which can later be chemically coupled to amine-reactive Cy dyes. In another example, the kit may include reagents for direct chemical linkage of Cy dyes to guanine residues for labeling PCR products.

In another embodiment, the kit may include one or more of the following reagents for sequencing the double-stranded PCR products: Taq DNA Polymerase, T4 Polynucleotide kinase, Exonuclease I (E. coli), sequencing primers, dNTPs, termination (deaza) mixes (mix G, mix A, mix T, mix C), DTT solution, and sequencing buffers.

The kit optionally includes instructions for using the kit in the selective amplification of mRNA targets. The kit can also be optionally provided with instructions for in vitro transcription of the amplified cDNA molecules and with instructions for labeling and hybridizing the in vitro transcription products to microarrays. The kit can also be provided with instructions for labeling and/or sequencing. The kit can also be provided with instructions for cloning the PCR products into an expression vector to generate an expression library representative of the transcriptome of the sample at the time the sample was taken.

In another aspect, the present invention provides methods of selectively amplifying a target population of nucleic acid molecules to generate selectively amplified cDNA molecules. The method according to this aspect of the invention comprises (a) providing a first population of oligonucleotides, wherein each oligonucleotide comprises a hybridizing portion and first PCR primer binding site located 5′ to the hybridizing portion, (b) annealing the first population of oligonucleotides to a sample comprising RNA templates isolated from a mammalian subject; (c) synthesizing cDNA from the RNA using a reverse transcriptase enzyme; (d) synthesizing double-stranded cDNA using a DNA polymerase and a second population of oligonucleotides, wherein each oligonucleotide comprises a hybridizing portion and a second PCR binding site located 5′ to the hybridizing portion, wherein the hybridizing portion is a member of the population of oligonucleotides comprising SEQ ID NOS:750-1498; and (e) purifying the double-stranded cDNA molecules. In some embodiments, the method further comprises PCR amplifying the double-stranded cDNA molecules. FIG. 1C shows a representative embodiment of the methods according to this aspect of the invention. As shown in FIG. 1C, in one embodiment of the method, the first primer mixture comprises a first PCR primer binding site (PBS#1) located 5′ to a hybridizing portion, wherein the hybridizing portion comprises a population of random 9mers.

In another embodiment, the present invention provides methods of selectively amplifying a target population of nucleic acid molecules to generate selectively amplified aDNA molecules. FIG. 1D shows a representative embodiment of the methods according to this aspect of the invention. As shown in FIG. 1D, the first primer mixture comprises a first PCR primer binding site (PBS#1) located 5′ to the hybridizing portion, wherein the hybridizing portion is a member of the population of oligonucleotides comprising SEQ ID NOS:1-749. The method further comprises PCR amplifying the double-stranded cDNA using thermostable DNA polymerase, a first PCR primer that binds to the first PCR primer binding site and a second PCR primer that binds to the second PCR primer binding site to generate amplified double-stranded DNA (aDNA). As shown in FIG. 1D, in some embodiments, the method further comprises the step of sequencing at least a portion of the aDNA.

The methods and reagents described herein are useful in the practice of this aspect of the invention. In accordance with this aspect of the invention, any DNA-dependent DNA polymerase may be utilized to synthesize second-strand DNA molecules from the first strand cDNA. For example, the Klenow fragment of DNA Polymerase I can be utilized to synthesize the second strand DNA molecules. The synthesis of second strand DNA molecules is primed using a second population of oligonucleotides comprising a hybridizing portion consisting of from 6 to 9 nucleotides and further comprising a defined sequence portion 5′ to the hybridizing portion.

The defined sequence portion may include any suitable sequence, provided that the sequence differs from the defined sequence contained in the first population of oligonucleotides. Depending on the choice of primer sequence, these defined sequence portions can be used, for example, to selectively direct DNA-dependent RNA synthesis from the second DNA molecule and/or to amplify the double-stranded cDNA template via DNA-dependent DNA synthesis.

Purification of Double-Stranded DNA Molecules. Synthesis of the second DNA molecules yields a population of double-stranded DNA molecules wherein the first DNA molecules are hybridized to the second DNA molecules, as shown in FIG. 1D. Typically, the double-stranded DNA molecules are purified to remove substantially all nucleic acid molecules shorter than 50 base pairs, including all or substantially all (i.e., typically more than 99%) of the second primers. Preferably, the purification method selectively purifies DNA molecules that are substantially double-stranded and removes substantially all unpaired, single-stranded nucleic acid molecules such as single-stranded primers. Purification can be achieved by any art-recognized means, such as by elution through a size-fractionation column. The purified second DNA molecules can then, for example, be precipitated and redissolved in a suitable buffer for the next step of the methods of this aspect of the invention.

Amplification of the Double-Stranded DNA Molecules. In the practice of the methods of this aspect of the invention, the double-stranded DNA molecules are utilized as templates that are enzymatically amplified using the polymerase chain reaction. Any suitable primers can be used to prime the polymerase chain reaction. Typically, two primers are used—one primer hybridizes to the defined portion of the first primer sequence (or to the complement thereof), and the other primer hybridizes to the defined portion of the second primer sequence (or to the complement thereof).

PCR Amplification Conditions. In general, the greater the number of amplification cycles during the polymerase chain reaction, the greater the amount of amplified DNA that is obtained. On the other hand, too many amplification cycles may result in randomly-biased amplification of the double-stranded DNA. Thus, in some embodiments, a desirable number of amplification cycles is between 5 and 40 amplification cycles, such as from 5 to 35, such as from 10 to 30 amplification cycles.

With regard to temperature conditions, typically a cycle comprises a melting temperature such as 95° C., an annealing temperature that varies from about 40° C. to 70° C., and an elongation temperature that is typically about 72° C. With regard to the annealing temperature, in some embodiments the annealing temperature is from about 55° C. to 65° C., more preferably about 60° C.

In one embodiment, amplification conditions for use in this aspect of the invention comprise 10 cycles of (95° C., 30 sec; 60° C., 30 sec; 72° C., 60 sec) then 20 cycles of (95° C., 30 sec; 60° C., 30 sec, 72° C., 60 sec (+10 sec added to the elongation step with each cycle)).

With regard to PCR reaction components for use in the methods of this aspect of the invention, dNTPs are typically present in the reaction in a range from 50 μM to 2000 μM dNTPs and, more preferably, from 800 to 1000 μM. MgCl₂ is typically present in the reaction in a range from 0.25 mM to 10 mM, and more preferably about 4 mM. The forward and reverse PCR primers are typically present in the reaction from about 50 nM to 2000 nM, and more preferably present at a concentration of about 1000 nM.

DNA Labeling. Optionally, the amplified DNA molecules can be labeled with a dye molecule to facilitate use as a probe in a hybridization experiment, such as a probe used to screen a DNA chip. Any suitable dye molecules can be utilized, such as fluorophores and chemiluminescers. An exemplary method for attaching the dye molecules to the amplified DNA molecules is provided in Example 5.

The methods according this aspect of the invention may be used, for example, for transcriptome profiling in a biological sample containing total RNA. In some embodiments, the amplified aDNA generated from cDNA using NSR priming in the first strand cDNA and anti-NSR priming in the second-strand synthesis produced in accordance with the methods of this aspect of the invention is labeled for use in gene expression experiments, thereby providing a hybridization based reagent that typically produces a lower level of background than amplified RNA generated from NSR-primed cDNA.

In some embodiments of this aspect of the invention, the defined sequence portion of the first and/or second primer binding regions further includes one or more restriction enzyme sites, thereby generating a population of amplified double-stranded DNA products having one or more restriction enzyme sites flanking the amplified portions. These amplified products may be used directly for sequence analysis or may be released by digestion with restriction enzymes and subcloned into any desired vector, such as an expression vector for further analysis. Sequence analysis of the PCR products may be carried out using any DNA sequencing method, such as, for example, the dideoxy chain termination method of Sanger, dye-terminator sequencing methods, or a high throughput sequencing method as described in U.S. Pat. No. 7,232,656 (Solexa), hereby incorporated by reference.

In another aspect, the invention provides a population of selectively amplified nucleic acid molecules comprising a representation of a target population of nucleic acid molecules within a population of RNA template molecules is a sample isolated from a mammalian subject, each amplified nucleic acid molecule comprising: a 5′ defined sequence portion flanking a member of the population of amplified nucleic acid sequences, and a 3′ defined sequence, wherein the population of selectively amplified sequences comprises amplified nucleic acid sequence corresponding to a target RNA molecule expressed in the mammalian subject, and is characterized by having the following properties with reference to the particular mammalian species: (a) having greater than 75% poly-adenylated and non-polyadenylated transcripts and having less than 10% ribosomal RNA (e.g., rRNA (18S or 28S) and mt-RNA).

The populations of selectively amplified nucleic acid molecules in accordance with this aspect of the invention can be generated using the methods of the invention described herein. The population of selectively amplified nucleic acid molecules may be cloned into an expression vector to generate a library. Alternatively, the population of selectively amplified nucleic acid molecules may be immobilized on a substrate to make a microarray of the amplification products. The microarray may comprise at least one amplification product immobilized on a solid or semi-solid substrate fabricated from a material selected from the group consisting of paper, glass, ceramic, plastic, polystyrene, polypropylene, nylon, polyacrylamide, nitrocellulose, silicon, metal, and optical fiber. An amplification product may be immobilized on the solid or semi-solid substrate in a two-dimensional configuration or a three-dimensional configuration comprising pins, rods, fibers, tapes, threads, beads, particles, microtiter wells, capillaries and cylinders.

The following examples merely illustrate the best mode now contemplated for practicing the invention, but should not be construed to limit the invention.

### Example 1

This Example describes the selection of a first population (Not-So-Random, “NSR”) of 749 6-mer oligonucleotides (SEQ ID NOS:1-749) that hybridizes to all or substantially all RNA molecules expressed in mammalian cells but that does not hybridize to nuclear ribosomal RNA (18S and 28S rRNA) or mitochondrial ribosomal RNA (12S and 16S mt-rRNA). A second population of anti-NSR oligonucleotides (SEQ ID NOS:750-1498) was also generated that is the reverse complement of the NSR oligos. The NSR oligo population may be used to prime first strand cDNA synthesis and the anti-NSR oligo population may be used to prime second strand cDNA synthesis.

Rationale:

Random 6-mers (N6) can anneal at every nucleotide position on a transcript sequence from the RefSeq database (represented as “nucleotide sequence”), as shown in FIG. 1A. After subtracting out the 6-mers whose reverse complements are a perfect match to nuclear ribosomal RNAs (18S and 28S rRNA) and mitochondrial ribosomal RNAs (12S and 16S mt-rRNA), the remaining NSR oligonucleotides (SEQ ID NOS:1-749) show a perfect match to every 4 to 5 nucleotides on nucleic acid sequences within the RefSeq database (represented as “nucleotide sequence”), as shown in FIG. 1B.

Methods:

All 4,096 possible 6-mer oligonucleotides were computed, wherein each nucleotide was A, T (or U), C, or G. The reverse complement of each 6-mer oligonucleotide was compared to the nucleotide sequences of 18S and 28S rRNAs, and to the nucleotide sequences of 12S and 16S mitochondrial rRNAs, as shown below in TABLE 1.

TABLE 1
RIBOSOMAL RNA

| | NCBI Reference Sequence | |
|---|---|---|
| Gene | Transcript Identifier, | Nucleotide |
| Symbol | accessed Sep. 5, 2007 | Coordinates |
| 12S | Genbank Ref # bJ01415.2 | nt648-1601 |
| 16S | Genbank Ref # bJ01415.2 | nt1671-3229 |
| 18S | Genbank Ref # bU13369.1 | nt3657-5527 |
| 28S | Genbank Ref # bU13369.1 | nt7935-12969 |

Reverse-complement 6-mer oligonucleotides having perfect matches to any of the human nuclear rRNA transcript sequences shown in TABLE 1, (which totaled 2,781) were eliminated. The reverse complements of 749 6-mers (SEQ ID NOS:1-749) did not perfectly match any portion of the rRNA transcripts. Matches to mitochondrial rRNA were also eliminated (566), leaving a total of 749 oligo 6-mers (4096 (all 6mers)-2782 (matches to euk-rRNAs)-566 (matches to mito-rRNA))=749 total.

The 749 6-mer oligonucleotides (SEQ ID NOS:1-749) that do not have a perfect match to any portion of the rRNA genes and mt-rRNA genes are referred to as “Not-So-Random” (“NSR”) primers. Thus the population of 749 6-mers (SEQ ID NOS:1-749) is capable of amplifying all transcripts except 18S, 28S, and mitochondrial rRNA (12S and 16S).

The population of NSR oligos (SEQ ID NO:1-749) may be used to prime first strand cDNA synthesis, as described in EXAMPLE 2, which may then be followed by second strand synthesis using either random primers, or anti-NSR primers.

As further described in EXAMPLE 2, a population of anti-NSR oligos (SEQ ID NOS:750-1498) may be used to prime second strand cDNA synthesis. As shown in FIG. 1C, first strand cDNA synthesis may be carried out using random primers, followed by second strand cDNA synthesis using anti-NSR primers. Alternatively, as shown in FIG. 1D, first strand cDNA synthesis may be carried out using NSR primers, followed by second strand cDNA synthesis using anti-NSR primers.

Applications to Other Types of RNA Samples. For gene profiling of mammalian cells other than human (e.g., rat, mouse), a similar approach may be carried out by subtracting out ribosomal nuclear rRNA of the genes corresponding to 18S and 28S, as well as subtracting out ribosomal mitochondrial rRNA of the genes corresponding to 12S and 16S from the respective mammalian species.

Gene profiling of plant cells may also be carried out by generating a population of Not-So-Random (NSR) primers that exclude chloroplast ribosomal RNA.

### Example 2

This Example shows that amplification of total RNA using NSR primers and anti-NSR primers selectively reduces priming of unwanted, non-target ribosomal sequences.

Methods:

To construct new primer libraries, primers were synthesized individually as follows:

A first population of NSR-timer primers (SEQ ID NOS:1-749) and a second population of anti-NSR-timer primers (SEQ ID NOS:750-1498) were generated as described in Example 1.

NSR for First Strand cDNA Synthesis. In some embodiments, the first primer set of NSR primers for use in first strand cDNA synthesis (SEQ ID NOS:1-749) further comprises the following 5′ primer binding sequence:

| PBS#1: | |
|---|---|
| 5′ TCCGATCTCT 3′ | (SEQ ID NO: 1499) |

covalently attached at the 5′ end (otherwise
referred to as “tailed”),

resulting in a population of oligonucleotides having the following configuration:

5′ PBS#1 (SEQ ID NO: 1499) +
NSR-6mer (SEQ ID NOS: 1-749) 3′

In another embodiment, a population of oligonucleotides was generated wherein each NSR-timer optionally included at least one spacer nucleotide (N) (where each N=A, G, C, or T) where (N) was located between the 5′PBS#1 and the NSR-timer. The spacer region may comprise from one nucleotide up to ten or more nucleotides (N=1 to 10), resulting in a population of oligonucleotides having the following configuration:

5′ PBS#1 (SEQ ID NO:1499)+(N₁₋₁₀)+NSR-6mer (SEQ ID NOS:1-749) 3′

Anti-NSR for Second Strand cDNA Synthesis. In some embodiments, the population of anti-NSR-timer primers for use in second strand cDNA synthesis (SEQ ID NOS:750-1498) further comprises the following 5′ primer binding sequence:

| PBS#2: | |
|---|---|
| 5′TCCGATCTGA 3′ | (SEQ ID NO: 1500) |

covalently attached at the 5′ end of the
anti-NSR-6mer primers (otherwise referred
to as “tailed”),

resulting in the following configuration:

5′ PBS#2 (SEQ ID NO: 1500) +
anti-NSR-6mer (SEQ ID NOS: 750-1498) 3′

In another embodiment, a population of oligonucleotides was generated wherein each anti-NSR-timer optionally included at least one spacer nucleotide (N) (where each N=A, G, C, or T) where (N) was located between the 5′PBS#2 and the anti-NSR-timer.

The spacer region may comprise from one nucleotide up to ten or more nucleotides (N=1 to 10), resulting in a population of oligonucleotides having the following configuration:

5′ PBS#2 (SEQ ID NO: 1500) + (N₁₋₁₀) +
anti-NSR-6mer (SEQ ID NOS: 750-1498) 3′

Forward and Reverse Primers (for PCR Amplification). The following forward and reverse primers were synthesized to amplify double-stranded cDNA generated using NSR-timers tailed with PBS#1 (SEQ ID NO:1499) and anti-NSR-timers tailed with PBS#2 (SEQ ID NO:1500).

NSR_F_SEQprimer 1: 5′ N₍₁₀₎TCCGATCTCT-3′ (SEQ ID NO:1501), where each N=G, A, C, or T.

NSR_R_SEQprimer 1: 5′ N₍₁₀₎TCCGATCTGA-3′ (SEQ ID NO:1502), where each N=G, A, C, or T.

In the embodiment described above, the 5′ most region of the forward primer (SEQ ID NO:1501) and reverse primer (SEQ ID NO:1502) each include a 10mer sequence of (N) nucleotides. In another embodiment, the 5′-most region of the forward primer (SEQ ID NO:1501) and reverse primer (SEQ ID NO:1502) each include more than 10 (N) nucleotides, such as at least 20 (N) nucleotides, at least 30 (N) nucleotides, or at least 40 (N) nucleotides to facilitate DNA sequencing of the amplified PCR products.

Control Primers. The following primers were used to amplify the control reactions amplified with random primer pools:

The following primer binding sites were added to random primers:

Y4F:

| | |
|---|---|
| 5′ CCACTCCATTTGTTCGTGTG 3′ | (SEQ ID NO: 1506) |
| Y4R: | |
| 5′ CCGAACTACCCACTTGCATT 3′ | (SEQ ID NO: 1507) |

The following primer binding sites with random primers (N=7 or N=9), or NSR primers:

Y4R-N7 (1st strand cDNA):

(SEQ ID NO: 1503)
5′ CCGAACTACCCACTTGCATTNNNNNNN 3′
[where N = A, G, C, or T]

Y4R-NSR (1st strand cDNA):
5′ CCGAACTACCCACTTGCATTN 3′ (SEQ ID NO:1504) covalently attached to NSR primers that include the core set of 6-mer NSR oligos with no perfect match to globin (alpha or beta), no perfect match to rRNA (18S, 28S).

Y4F-N9 (2nd strand cDNA synthesis):

(SEQ ID NO: 1505)
5′ CCACTCCATTTGTTCGTGTGNNNNNNNNN 3′
[where N = A, G, C, or T]

Y4F

| | |
|---|---|
| 5′ CCACTCCATTTGTTCGTGTG 3′ | (SEQ ID NO: 1506) |
| Y4R | |
| 5′ CCGAACTACCCACTTGCATT 3′ | (SEQ ID NO: 1507) |

Other Optional Primer Pool Configurations. Additional primers that could be used as primer binding sites covalently attached to the NSR pool in order to add transcriptional promoters to the amplified cDNA product:

T7:
(SEQ ID NO: 1508)
5′ AATTAATACGACTCACTATAGGGAGA 3′
SP6:
(SEQ ID NO: 1509)
5′ ATTTAGGTGACACTATAGAAGNG 3′
T3:
(SEQ ID NO: 1510)
5′AATTAACCCTCACTAAAGGGAGA 3′

Primer Pool Configurations Used to Amplify RNA. Primers were synthesized individually as described above and pooled in the following configuration, then the primer pools were used to generate libraries of amplified nucleic acids from total RNA as described below.

TABLE 2
PRIMER POOL CONFIGURATIONS

| | Pool Components | | | | 5′ Primer |
|---|---|---|---|---|---|
| | (includes all | Number of | | | Binding |
| | expressed RNA | individual | | | Sequence |
| | except for | sequences | | | (covalently |
| Reference ID | those listed) | in Pool | Description of Pool | SEQ ID NO: | attached) |
| saNSR#1 pool | NSR-6mers - | 510 | core set of 6-mer NSR | SEQ ID NO: | PBS#1 |
| | (R, M, G) | | oligos with no perfect | 1-510, with a | (SEQ ID |
| | | | match to rRNA (18S, | spacer (N = A, G, | NO: 1499) |
| | | | 28S), mt-RNA (12S, | C, or T) located | |
| | | | 16S) or globin (alpha | between PBS#1 | |
| | | | or beta) | and NSR-6mer | |
| saNSR#2 pool | NSR-6mers - | 403 | core set of 6-mer NSR | control set, | SEQ ID |
| | (G, R) | | oligos with perfect | (sequences not | NO: 1499 |
| | | | match to mt-rRNA, but | provided) | |
| | | | not globin or rRNA | | |
| saNSR#3 pool | NSR-6mers - | 239 | core set of 6-mer NSR | SEQ ID NO: | PBS#1 |
| | (M, R) | | oligos with perfect | 511-749 with a | (SEQ ID |
| | | | match to globins, but | spacer (N = A, G, | NO: 1499) |
| | | | not mt-rRNA or rRNA | C, or T) located | |
| | | | | between PBS#1 | |
| | | | | and NSR-6mer | |
| saNSR #4 | NSR-6mers - | 163 | core set of 6-mer NSR | control set, | SEQ ID |
| pool | (R) | | oligos with perfect | (sequences not | NO: 1499 |
| | | | match to mt-rRNA and | shown) | |
| | | | globin, but not to | | |
| | | | rRNA | | |
| sa-antiNSR#5 | anti-NSR-6mers - | 510 | core set of 6-mer NSR | SEQ ID NO: | PBS#2 |
| pool | (R, M, G) | | oligos with no perfect | 750-1259 with a | (SEQ ID |
| | | | match to rRNA (18S, | spacer (N = A, G, | NO: 1500) |
| | | | 28S), mt-RNA (12S, | C, or T) located | |
| | | | 16S) or globin (alpha | between PBS#2 | |
| | | | or beta); | and | |
| | | | | anti-NSR-6mer | |
| sa-antiNSR#6 | anti-NSR-6mers - | 403 | core set of 6-mer | control set, | SEQ ID |
| pool | (G, R) | | anti-NSR oligos with | (sequences not | NO: 1500 |
| | | | perfect match to | shown) | |
| | | | mt-rRNA, but not | | |
| | | | globin or rRNA | | |
| sa-antiNSR#7 | anti-NSR-6mers - | 239 | core set of 6-mer | SEQ ID NO: | PBS#2 |
| pool | (M, R) | | antiNSR oligos with | 1260-1499 with | (SEQ ID |
| | | | perfect match to | a spacer (N = A, | NO: 1500) |
| | | | globins, but not | G, C, or T) | |
| | | | mt-rRNA or rRNA | located between | |
| | | | | PBS#2 and | |
| | | | | anti-NSR-6mer | |
| sa-antiNSR#8 | anti-NSR-6mers - | 163 | core set of 6-mer | control set, | SEQ ID |
| pool | (R) | | anti-NSR oligos with | (sequences not | NO: 1500 |
| | | | perfect match to | shown) | |
| | | | mt-rRNA and globin, | | |
| | | | but not to rRNA | | |

PM = perfect match at 3′-most 6nt of primer
R = rRNA (18S or 28S)
M = mt-rRNA (12S or 16S)
G = globin (HBA1, HBA2, HBB, HBD, HBG1, HBG2)

TABLE 3
PRIMER SETS FOR USE IN RNA AMPLIFICATION EXPERIMENT

| Reference ID | Process | Amount (μL) | Description | SEQ ID NO: |
|---|---|---|---|---|
| saNSR#1 pool | 1st strand cDNA | 510 μL total | 510 μL of saNSR#1 | SEQ ID NOS: |
| | synthesis | | pool only | 1-510, with a |
| | | | | spacer (N = A, G, C, |
| | | | | or T) located |
| | | | | between PBS#1 and |
| | | | | NSR-6mer |
| saNSR#1 pool + | 1st strand cDNA | 913 μL total | 510 μL of saNSR#1 | control set |
| saNSR#2 pool | synthesis | | pool combined with | |
| | | | 403 μL of saNSR#2 | |
| | | | pool | |
| saNSR#1 pool + | 1st strand cDNA | 749 μL total | 510 μL of saNSR#1 | SEQ ID NOS: |
| saNSR#3 pool | synthesis | | pool combined with | 1-749, with a |
| | | | 239 μL of NSR#3 pool | spacer (N = A, G, C, |
| | | | | or T) located |
| | | | | between PBS#1 and |
| | | | | NSR-6mer |
| saNSR#1 pool + | 1st strand cDNA | 673 μL total | 510 μL of saNSR#1 | control set |
| saNSR#4 pool | synthesis | | pool combined with | |
| | | | 163 μL of saNSR#4 | |
| | | | pool | |
| sa-anti-NSR#5 | 2nd strand | 510 μL total | 510 μL of sa-antiNSR#5 | SEQ ID NOS: |
| pool | cDNA synthesis | | pool only | 750-1259 with a |
| | | | | spacer (N = A, G, C, |
| | | | | or T) located |
| | | | | between PBS#2 and |
| | | | | anti-NSR-6mer |
| sa-anti-NSR#5 | 2nd strand | 913 μL total | 510 μL of | control set |
| pool + | cDNA synthesis | | sa-anti-NSR#5 pool | |
| sa-anti-NSR#6 | | | combined with 403 μL | |
| pool | | | of sa-anti-NSR#6 pool | |
| sa-anti-NSR#5 | 2nd strand | 749 μL total | 510 μL of | SEQ ID NOS: |
| pool + | cDNA synthesis | | sa-anti-NSR#5 pool | 750-1499 with a |
| sa-anti-NSR#7 | | | combined with 239 μL | spacer (N = A, G, C, |
| pool | | | of sa-anti-NSR#7 pool | or T) located |
| | | | | between PBS#2 and |
| | | | | anti-NSR-6mer |
| sa-anti-NSR#5 | 2nd strand | 673 μL total | 510 μL of | control set |
| pool + | cDNA synthesis | | sa-anti-NSR#5 pool | |
| sa-anti-NSR#8 | | | combined with 163 μL | |
| pool | | | of sa-anti-NSR#8 pool | |

cDNA Synthesis and PCR Amplification. The protocol involved a three-step amplification approach as follows: (1) first strand cDNA was generated from RNA using reverse transcription that was primed with NSR primers comprising a first primer binding site (PBS#1) to generate NSR primed first strand cDNA; (2) second strand cDNA synthesis was primed with anti-NSR primers comprising a second primer binding site (PBS#2); and (3) the synthesized cDNA was PCR amplified using forward and reverse primers that bind to the first and second primer binding sites to generate amplified DNA (aDNA).

TABLE 4
PRIMERS USED FOR FIRST AND SECOND STRAND SYNTHESIS

| | 1st Strand Primer Pool | | RNA Template | |
|---|---|---|---|---|
| Reaction | (+Reverse Transcriptase) | 2nd Strand Primer Pool | (1 μL of 1 μg/uL | |
| ID | 100 μM | (+Klenow) | Total RNA) | Method |
| 1 | saNSR#1 pool | sa-anti-NSR#5 pool | Jurkat-1 | RT-PCR |
| 2 | saNSR#1 pool + | sa-anti-NSR#5 pool + | Jurkat-1 | RT-PCR |
| | saNSR#2 pool | sa-anti-NSR#6 pool | | |
| 3 | saNSR#1 pool + | sa-anti-NSR#5 pool + | Jurkat-1 | RT-PCR |
| | saNSR#3 pool | sa-anti-NSR#7 pool | | |
| 4 | saNSR#1 pool + | sa-anti-NSR#5 pool + | Jurkat-1 | RT-PCR |
| | saNSR#4 pool | sa-anti-NSR#8 pool | | |
| 5 | Y4R-NSR | Y4F-N9 | Jurkat-1 | RT-PCR |
| 6 | Y4R-NSR | Y4F-N9 | Jurkat-1 | RT-PCR |
| 7 | Y4-N7 | Y4F-N9 | Jurkat-1 | RT-PCR |
| 8 | N8 | None | Jurkat-1 | RT |
| 9 | saNSR#1 pool | sa-anti-NSR#5 pool | Jurkat-2 | RT-PCR |
| 10 | saNSR#1 pool + | sa-anti-NSR#5 pool + | Jurkat-2 | RT-PCR |
| | saNSR#2 pool | sa-anti-NSR#6 pool | | |
| 11 | saNSR#1 pool + | sa-anti-NSR#5 pool + | Jurkat-2 | RT-PCR |
| | saNSR#3 pool | sa-anti-NSR#7 pool | | |
| 12 | saNSR#1 pool + | sa-anti-NSR#5 pool + | Jurkat-2 | RT-PCR |
| | saNSR#4 pool | sa-anti-NSR#8 pool | | |
| 13 | Y4R-NSR | Y4F-N9 | Jurkat-2 | RT-PCR |
| 14 | Y4R-NSR | Y4F-N9 | Jurkat-2 | RT-PCR |
| 15 | Y4-N7 | Y4F-N9 | Jurkat-2 | RT-PCR |
| 16 | N8 | None | Jurkat-2 | RT |
| 17 | saNSR#1 pool | sa-antiNSR#5 pool | K562 | RT-PCR |
| 18 | saNSR#1 pool + | sa-anti-NSR#5 pool + | K562 | RT-PCR |
| | saNSR#2 pool | sa-anti-NSR#6 pool | | |
| 19 | saNSR#1 pool + | sa-anti-NSR#5 pool + | K562 | RT-PCR |
| | saNSR#3 pool | sa-anti-NSR#7 pool | | |
| 20 | saNSR#1 pool + | sa-anti-NSR#5 pool + | K562 | RT-PCR |
| | saNSR#4 pool | sa-anti-NSR#8 pool | | |
| 21 | Y4R-NSR | Y4F-N9 | K562 | RT-PCR |
| 22 | Y4R-NSR | Y4F-N9 | K562 | RT-PCR |
| 23 | Y4-N7 | Y4F-N9 | K562 | RT-PCR |
| 24 | N8 | None | K562 | RT |

Reaction Conditions:

Total RNA was obtained from Ambion, Inc. (Austin, Tex.), for the cell lines Jurkat (T lymphocyte, ATCC No. TIB-152) and K562 (chronic myelogenous leukemia, ATCC No. CCL-243).

First Strand Reverse Transcription:

First strand reverse transcription was carried out as follows:

Combine:
1 μl of 1 μg/μl Jurkat total RNA template (obtained from Ambion, Inc. (Austin, Tex.)).
2 μl of 100 μM stock NSR primer pool (as described in Table 2)
7 μl H₂O to a final volume of 10 μl.

Mixed and incubated at 70° C. for 5 minutes, snap chilled on ice.

Added 10 μl of RT cocktail (prepared on ice) containing:
4 ul 5× First Strand Buffer (250 mM Tris-HCL, pH 8.3, 375 mM KCl, 15 mM MgCl₂)
1.6 μl 25 mM dNTP (high) or 1.0 ul 10 mM dNTP (low)
1 μl H₂O
1 μl 0.1 M DTT
1 μl RNAse OUT (Invitrogen)
1 μl MMLV reverse transcriptase (200 units/0) (Superscript III™ (SSIII), Invitrogen Corporation, Carlsbad, Calif.)

The sample was mixed, incubated at 23° C. for 10 minutes, transferred to a 40° C. pre-warmed thermal cycler (to provide a “hot start”), and the sample was then incubated at 40° C. for 30 minutes, 70° C. for 15 minutes, and chilled to 4° C.

1 μl of RNAse H (1-4 units/0) was then added and the sample was incubated at 37° C. for 20 minutes, then heated to 95° C. for 5 minutes, and snap-chilled at 4° C.

Second Strand Synthesis:

A second strand synthesis cocktail was prepared as follows:
10 μl 10× Klenow Buffer
4 μl anti-NSR Primer (100 μM)
5.0 μl 10 mM dNTPs
56.7 μl H₂O
0.33 μl Klenow enzyme (5 U/μl)

80 μl of the second strand synthesis cocktail was added to the 20 μl first strand template reaction mixture, mixed and incubated at 37° C. for 30 minutes, then snap-chilled at 4° C.

cDNA Purification:

The resulting double-stranded cDNA was purified using Spin Cartridges obtained from Ambion (Message Amp™ II aRNA Amplification Kit, Ambion Cat #AM1751) and buffers supplied in the kit according to the manufacturer's directions. A total volume of 30 μl was eluted from the column, of which 20 μl was used for follow-on PCR.

PCR Amplification:

The following mixture was added to 1 μl of purified cDNA template (diluted 1:5):
10 μl 5× Roche Expand Plus PCR Buffer
2.5 μl 110 mM dNTPS
2.5 μl Forward PCR Primer (10 μM stock) (SEQ ID NO:1501)
2.5 μl Reverse PCR Primer (10 μM stock) (SEQ ID NO:1502)
0.5 μl Taq DNA polymerase enzyme
27 μl H₂O
4 μl 25 mM MgCl₂

PCR Amplification Conditions:

PCR Program #1:

94° C. for 2 minutes

94° C. for 10 seconds

8 cycles of:
60° C. for 10 sec
72° C. for 60 sec
72° C. for 60 sec

94° C. for 15 sec

17 cycles of:
60° C. for 30 sec
72° C. for 60 sec+10 sec/cycle

72° C. for 5 minutes to polish and chilled at 4° C.

PCR program #2:

94° C. for 2 minutes

94° C. for 10 seconds

2 cycles of:
40° C. for 10 sec
72° C. for 60 sec
72° C. for 60 sec
94° C. for 10 seconds

8 cycles of:
60° C. for 30 sec
72° C. for 60 sec
72° C. for 60 sec
94° C. for 15 sec

15 cycles of:
60° C. for 30 sec
72° C. for 60 sec+10 sec/cycle

72° C. for 5 minutes to polish and chilled at 4° C.

Results of cDNA Synthesis:

The results were analyzed in terms of (1) measuring amplified DNA “aDNA” yield; (2) evaluation of an aliquot of the aDNA on an agarose gel to confirm that the population of species in the cDNA was equally represented; and (3) measuring the level of amplification of selected reporter genes by qPCR (as described in Example 3).

The PCR products were analyzed on 2% agarose gels. A DNA smear between 100-1000 by was observed for both control reactions and test conditions using the PCR amplification program #2, indicating successful cDNA synthesis of a plurality of RNA species and PCR amplification. With PCR amplification program #1, the control reactions were successful as determined by the presence of a DNA smear in the 100-100 by range; however, none of the test conditions amplified into a DNA smear. Instead, a low molecular weight fragment was observed that likely resulted from primer dimers (unpurified PCR product). Therefore, these results indicate that low temperature annealing (40° C.) is important for PCR amplification with short (10 nt) amplification tails.

It was also determined that high dNTP concentration (25 mM) during first strand cDNA synthesis increased specificity of the cDNA product as compared to low dNTP concentration (10 mM) dNTP (data not shown).

It was further determined that RNAse H treatment reduced the amount of contamination from amplified rRNA if the NSR primer pool was used only for first strand cDNA synthesis followed by random primed second strand synthesis. However, when NSR primers were used to prime the first strand synthesis, followed by the use of anti-NSR primers to prime the second strand synthesis, then RNAse treatment was not found to affect specificity of the resulting cDNA product. Although not important for increasing specificity, RNAse may be added to second strand cDNA synthesis using anti-NSR primers to improve efficiency of the reaction by making the cDNA more available as a template during the Klenow reaction.

In summary, it was found that the use of anti-NSR primers during second strand synthesis provided several unexpected advantages for selective amplification of target nucleic acid molecules. For example, it was unexpectedly found that the magnitude of rRNA depletion during second strand synthesis using anti-NSR primers was nearly identical to the magnitude of rRNA depletion observed using NSR primers during reverse transcription. In addition, it was an unexpected result that priming specificity during second strand synthesis was achieved under standard reaction conditions using Klenow enzyme. These results indicate that short oligonucleotides can be used to specifically prime DNA synthesis using a variety of polymerases and nucleic acid templates, however, the reaction conditions that dictate priming specificity may be enzyme-specific.

### Example 3

This Example shows that the 749 NSR 6-mers (SEQ ID NOS:1-749) (that each have PBS#1 (SEQ ID NO:1499 plus N spacer) covalently attached at the 5′ end) for first strand cDNA synthesis followed by the 749 anti-NSR 6-mers (SEQ ID NOS:750-1498) (that each have PBS#2 (SEQ ID NO:1500 plus N spacer) covalently attached at the 5′ end) prime the amplification of a substantial fraction of the transcriptome present in a sample containing total RNA.

Methods:

Following PCR amplification as described in Example 2, each PCR reaction was purified using the Qiagen MinElute spin column. The column was washed with 80% ethanol and eluted with 20 μL of elution buffer. The yield was quantitated with UV/VIS spectrometer using the NanoDrop instrument. Samples were then diluted and characterized by quantitative PCR (qPCR) using the following assays:

Duplicate measurements of 2 μl of cDNA were made in 10 μl final reaction volumes by quantitative PCR (qPCR) in a 384-well optical PCR plate using a 7900 HT PCR instrument (Applied Biosystems, Foster City, Calif.). qPCR was performed using ABI TaqMan® assays using the probes shown below in TABLE 5 and TABLE 6 using the manufacturer's recommended conditions.

TABLE 5
REPORTER GENE ASSAYS FOR JURKAT CELLS

| | | | | FAM |
|---|---|---|---|---|
| | | Forward | Reverse | reporter |
| Target | ABI Assay probe | Primer | Primer | primer |
| STMN1 | Hs01027516_g1 | Not | NR | NR |
| stathmin 1/ | | Relevant | | |
| oncoprotein 18 | | (NR) | | |
| PPIA | Hs99999904_m1 | NR | NR | NR |
| peptidylprolyl | | | | |
| isomerase A | | | | |
| (cyclophilin A) | | | | |
| EIF3S3 | Hs00186779_m1 | NR | NR | NR |
| eukaryotic | | | | |
| translation initiation | | | | |
| factor 3, subunit | | | | |
| 3 gamma, 40 kDa | | | | |
| NUCB2 | Hs00172851_m1 | NR | NR | NR |
| nucleobindin 2 | | | | |
| SRP14 | Hs01923965_u1 | NR | NR | NR |
| signal recognition | | | | |
| particle 14 kDa | | | | |
| (homologous Alu | | | | |
| RNA binding | | | | |
| protein) | | | | |
| TRIM63 | Hs00761590 | NR | NR | NR |
| DBN1 | Hs00365623 | NR | NR | NR |
| CDCA7 | Hs00230589_m1 | NR | NR | NR |
| GAPDH | Hs99999905 | NR | NR | NR |
| Actin (ACTB) | Hs99999903 | NR | NR | NR |
| 18s rRNA | Hs99999901_s1 | NR | NR | NR |

| | ABI Assay | | | FAM |
|---|---|---|---|---|
| Target | probe | Forward Primer | Reverse Primer | reporter primer |
| R28S_3-ANY | custom | GGTTCGCCCCGAGAGA | GGACGCCGCCGGAA | CCGCGACGCTTTCCAA |
| | | (SEQ ID NO: 1511) | (SEQ ID NO: 1512) | (SEQ ID NO: 1513) |
| 28S.4-JUN | custom | GTAGCCAAATGCCTCGTCATC | CAGTGGGAATCTCGTTCATCC | ATGCGCGTCACTAATTA |
| | | (SEQ ID NO: 1514) | ATT | (SEQ ID NO: 1516) |
| | | | (SEQ ID NO: 1515) | |
| 28S-7-ANY | custom | CCGAAACGATCTCAACCTATT | GCTCCACGCCAGCGA | CCGGGCTTCTTACCC |
| | | CTCA | (SEQ ID NO: 1518) | (SEQ ID NO: 1519) |
| | | (SEQ ID NO: 1517) | | |
| 28S-8-ANY | custom | GCGGGTGGTAAACTCCATCTA | CCCTTACGGTACTTGTTGACT | TCGTGCCGGTATTTAG |
| | | AG | ATCG | (SEQ ID NO: 1522) |
| | | (SEQ ID NO: 1520) | (SEQ ID NO: 1521) | |
| 18S-1-ANY | custom | GGTGACCACGGGTGACG | GGATGTGGTAGCCGTTTCTCA | TCCCTCTCCGGAATCG |
| | | (SEQ ID NO: 1523) | (SEQ ID NO: 1524) | (SEQ ID NO: 1525) |
| 16S-1-ANY | custom | ACCAAGCATAATATAGCAAG | TGGCTCTCCTTGCAAAGTTAT | CCTTCTGCATAATGAATTAA |
| | | GACTAACC | TTCT | (SEQ ID NO: 1528) |
| | | (SEQ ID NO: 1526) | (SEQ ID NO: 1527) | |
| 12S-1-ANY | custom | GACAAGCATCAAGCACGCA | CTAAAGGTTAATCACTGCTGT | CAATGCAGCTCAAAACG |
| | | (SEQ ID NO: 1529) | TTCCC | (SEQ ID NO: 1531) |
| | | | (SEQ ID NO: 1530) | |
| 12S-2-ANY | custom | GTCGAAGGTGGATTTAGCAGT | TGTACGCGCTTCAGGGC | CCTGTTCAACTAAGCACTCTA |
| | | AAAC | (SEQ ID NO: 1533) | (SEQ ID NO: 1534) |
| | | (SEQ ID NO: 1532) | | |
| hs16S-2 | custom | AAGCGTTCAAGCTCAACACC | GGTCCAATTGGGTATGAGGA | |
| | | (SEQ ID NO: 1535) | (SEQ ID NO: 1536) | |
| hs16S-3 | custom | GCATAAGCCTGCGTCAGATT | GGTTGATTGTAGATATTGGGC | |
| | | (SEQ ID NO: 1537) | TGT | |
| | | | (SEQ ID NO: 1538) | |
| hsHST1_H2AH | custom | TACCTGACCGCTGAGATCCT | AGCTTGTTGAGCTCCTCGTC | |
| | | (SEQ ID NO: 1539) | (SEQ ID NO: 1540) | |
| hsNC_7SK | custom | GACATCTGTCACCCCATTGA | CTCCTCTATCGGGGATGGTC | |
| | | (SEQ ID NO: 1541) | (SEQ ID NO: 1542) | |
| hsNC_7SL1 | custom | GGAGTTCTGGGCTGTAGTGC | GTTTTGACCTGCTCCGTTTC | |
| | | (SEQ ID NO: 1543) | (SEQ ID NO: 1544) | |
| hsNC_BC200 | custom | GCTAAGAGGCGGGAGGATAG | GGTTGTTGCTTTGAGGGAAG | |
| | | (SEQ ID NO: 1545) | (SEQ ID NO: 1546) | |
| hsNC_HY1 | custom | GCTGGTCCGAAGGTAGTGAG | ATGCCAGGAGAGTGGAAACT | |
| | | (SEQ ID NO: 1547) | (SEQ ID NO: 1548) | |
| hsNC_HY3 | custom | TCCGAGTGCAGTGGTGTTTA | GTGGGAGTGGAGAAGGAACA | |
| | | (SEQ ID NO: 1549) | (SEQ ID NO: 1550) | |
| hsNC_HY4 | custom | GGTCCGATGGTAGTGGGTTA | AAAAAGCCAGTCAAATTTAG | |
| | | (SEQ ID NO: 1551) | CA | |
| | | | (SEQ ID NO: 1552) | |
| hsNC_U4B1 | custom | TGGCAGTATCGTAGCCAATG | CTGTCAAAAATTGCCAATGC | |
| | | (SEQ ID NO: 1553) | (SEQ ID NO: 1554) | |
| hsNC_U6A | custom | CGCTTCGGCAGCACATATAC | AAAATATGGAACGCTTCACG | |
| | | (SEQ ID NO: 1555) | A | |
| | | | (SEQ ID NO: 1556) | |

TABLE 6
REPORTER GENE PROBES

REPORTER

| Assay Name | FAM | SYBR | 1/df |
|---|---|---|---|
| NUCB2 | + | | 10 |
| 18s (Hs99999901_s1) | + | | 1000 |
| 18S-1 | + | | 1000 |
| 18S-4 | + | | 1000 |
| 28S-3 | + | | 1000 |
| 28S-4 | + | | 1000 |
| 28S-7 | + | | 1000 |
| 28S-8 | + | | 1000 |
| 12S-1 | + | | 1000 |
| 12S-2 | + | | 1000 |
| 16S-1 | + | | 1000 |
| hs16S-2 | | + | 1000 |
| hs16S-3 | | + | 1000 |
| hsHST1_H2AHfwd | | + | 1000 |
| hsNC_7SKfwd | | + | 1000 |
| hsNC_7SL1fwd | | + | 1000 |
| NUCB2 | + | | 10 |
| PPIA | + | | 10 |
| SRP14 | + | | 10 |
| STMN1 | + | | 10 |
| TRIM63 | + | | 10 |
| ACTB | + | | 10 |
| CDCA7 | + | | 10 |
| DBN1 | + | | 10 |
| EIF3S3 | + | | 10 |
| GAPDH | + | | 10 |
| hsNC_BC200fwd | | + | 10 |
| hsNC_HY1fwd | | + | 10 |
| hsNC_HY3fwd | | + | 1000 |
| hsNC_HY4fwd | | + | 1000 |
| hsNC_U4B1fwd | | + | 10 |
| hsNC_U6Afwd | | + | 10 |

Following qPCR, the results table was exported to Excel (Microsoft Corp., Redmond, Wash.) and quantitative analysis for samples was regressed from the raw data (abundance=10^{[(Ct-5)/-3.4]}).

Results:

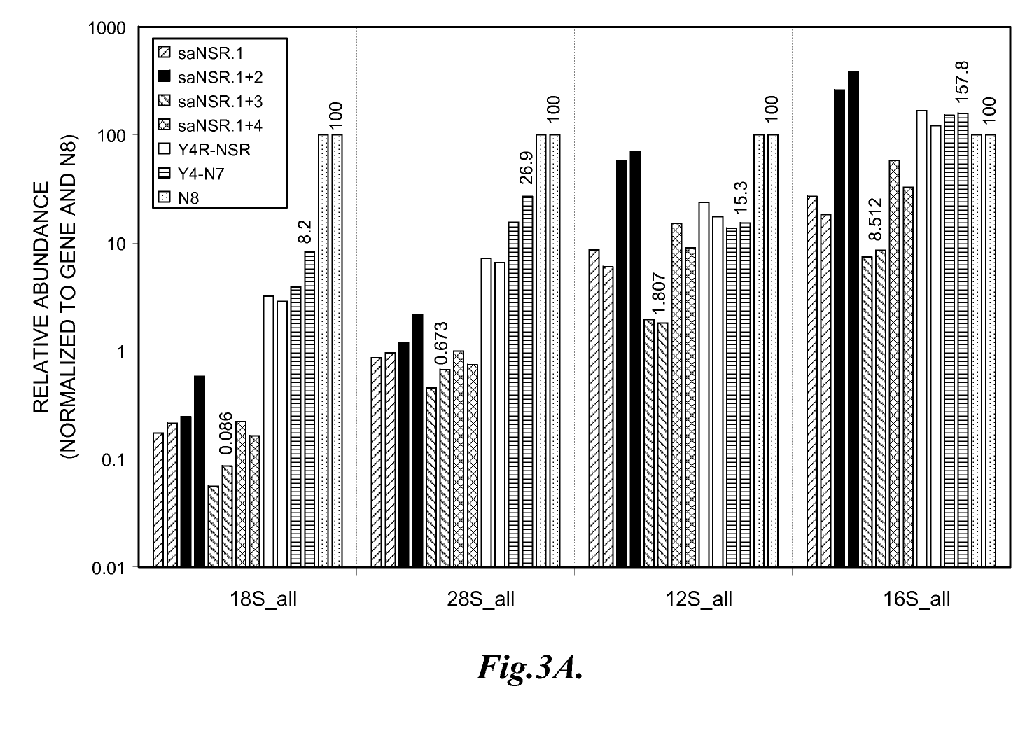

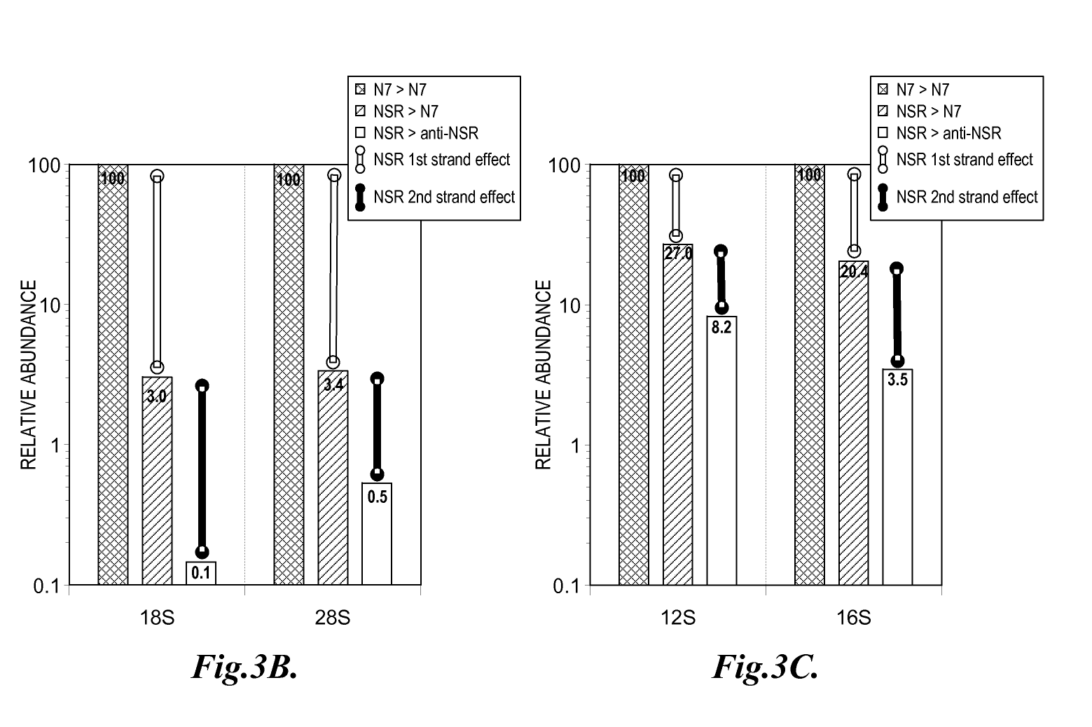

FIG. 3A is a histogram plot on a logarithmic scale showing the relative abundance of 18S, 28S, 12S and 16S (normalized to gene and N8) for first strand cDNA synthesis generated using various NSR pools as shown in TABLE 4 as compared to unamplified cDNA generated using random primers (N8=100%). As shown in FIG. 3A, the cDNA generated using the primer pool with NSR#1+NSR#3 (NSR-6mers that do not hybridize to mt-rRNA or rRNA) for first strand cDNA synthesis and the primer pool anti-NSR#5 and anti-NSR#7 for second strand synthesis showed a substantial reduction in abundance of rRNA (0.086% 18S; 0.673% 28S) and a reduced abundance of mt-rRNA (1.807% 12S; and 8.512% 16S) as compared to cDNA generated with random 8-mers.

FIG. 3B graphically illustrates the relative levels of abundance of nuclear ribosomal RNA (18S or 28S) in control cDNA amplified using random primers (N7) in both first strand and second strand synthesis (N7>N7=100% 18S, 100% 28S) as compared to cDNA amplified using NSR-6mer primers (SEQ ID NOS:1-749) in the first strand followed by random primers (N7) in the second strand (NSR-6mer>N7=3.0% 18S, 3.4% 28S), and as compared to cDNA amplified using NSR-6mer primers (SEQ ID NOS:1-749) in the first strand followed by anti-NSR-6mer primers (SEQ ID NOS:750-1498) in the second strand (NSR-6mer>anti-NSR-6mer=0.1% 18S, 0.5% 28S). The results in FIG. 3C show a similar trend when measuring mitochondrial rRNA, with N7>N7=100% 12S, or 16S; NSR-6mer>N7=27% 12S, 20.4% 16S; and NSR-6mer>anti-NSR-6mer=8.2% 12S, 3.5% 16S.

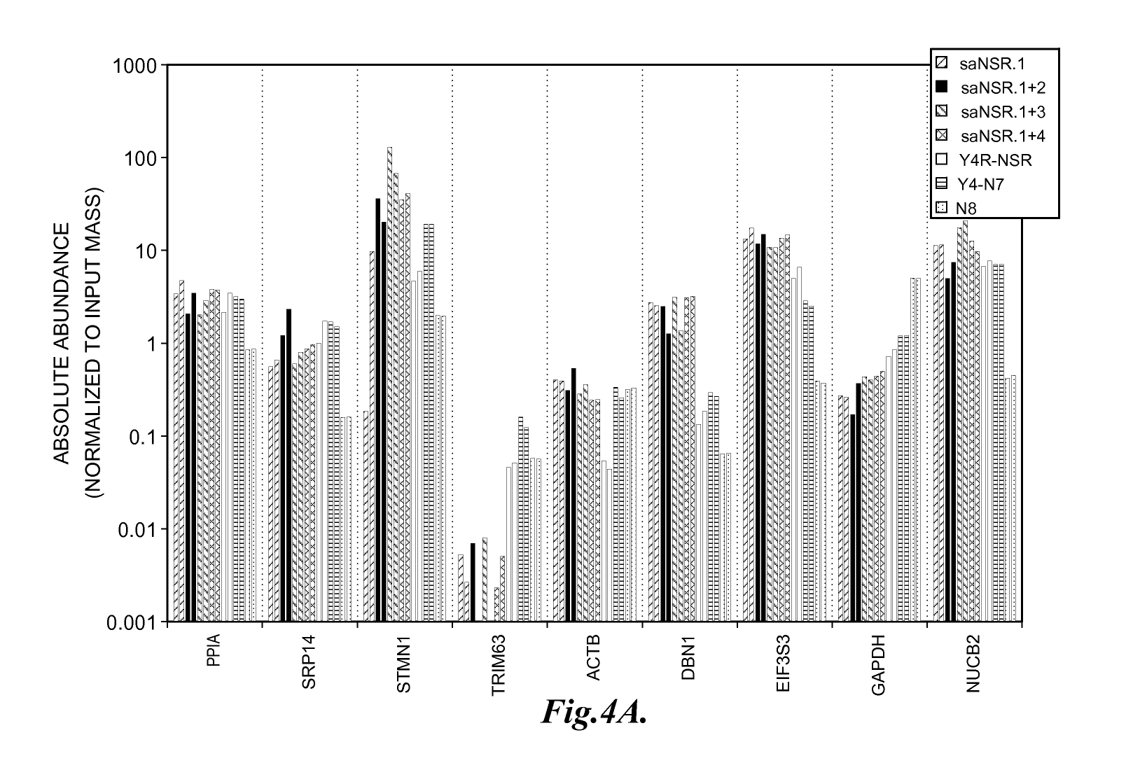

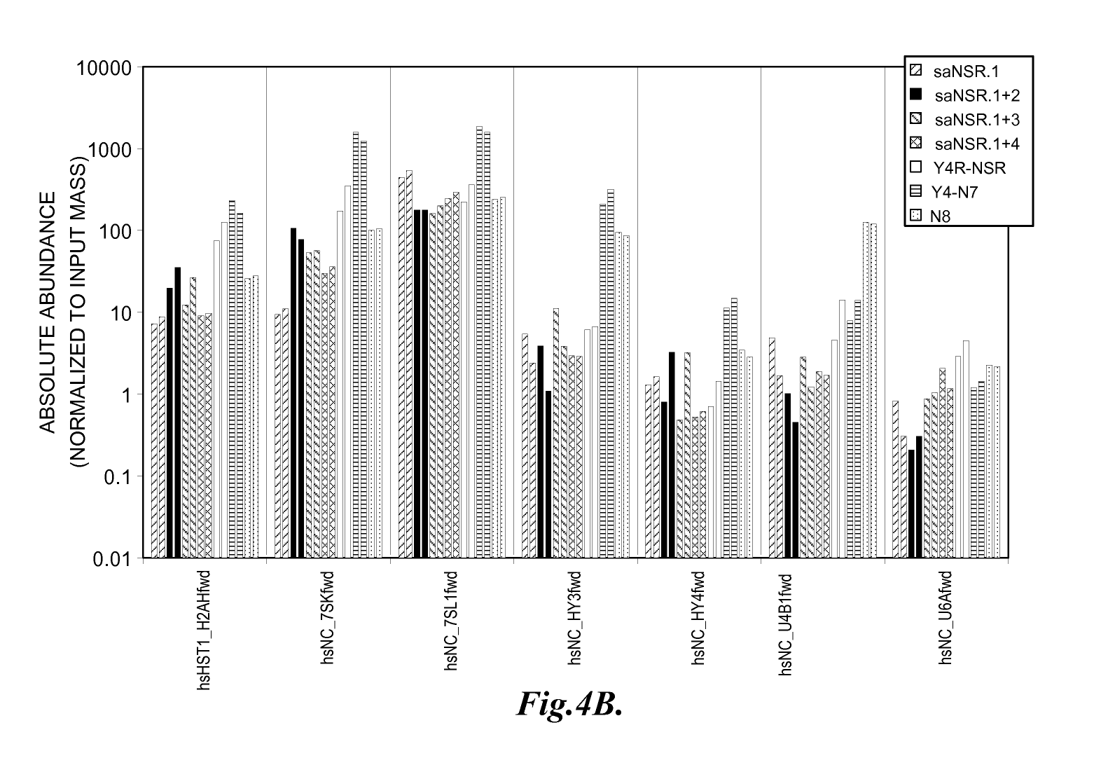

In order to determine if the PCR amplified aDNA generated from the cDNA synthesized using the various NSR and anti-NSR pools preserved the target gene expression profiles present in the corresponding cDNA, quantitative PCR analysis was conducted with nine randomly chosen TaqMan reagents, detecting the following genes: PPIA, SRP14, STMN1, TRIM63, ACTB, DBN1, EIFS3, GAPDH, and NUCB2. As shown in TABLE 7 and FIG. 4A, measurable signal was measured for the nine genes assayed in both NSR and anti-NSR primed cDNA and aDNA generated therefrom (as determined from 10 μl cDNA template input).

TABLE 7
QUANTITATIVE PCR ANALYSIS

| | | 1st strand | | | | | | | | | | | | |
|---|---|---|---|---|---|---|---|---|---|---|---|---|---|---|
| | | Primer Pool | | | | | | | | | | | | |
| Sam- | | (+Reverse | 2nd strand | | | | | | | | | | | |
| ple | | Tran- | Primer Pool | | Input Adjusted Abundance | | | | | | | | | |
| ID | ng/ul | scriptase) | (+Klenow) | RNA | NUCB2¹ | 18S³ | 18S-1² | 28S-3² | 28S-4² | 28S-7² | 28S-8² | 12S-1² | 12S-2² | 16S-1² |
| 1 | 76.5 | saNSR.1 | sa.anti-NSR#5 | Jurkat 1 | 11.4 | 52.9 | 195.0 | 349.1 | 800.8 | 989.2 | 612.5 | 798.8 | 216.0 | 108.1 |
| | | pool | pool | | | | | | | | | | | |
| 2 | 73.1 | saNSR.1 | sa.anti-NSR#5 | Jurkat 1 | 5.0 | 55.9 | 238.2 | 335.5 | 616.0 | 1066.5 | 715.2 | 1478.0 | 3671.0 | 863.7 |
| | | pool + | pool + | | | | | | | | | | | |
| | | 2 pool | sa.anti-NSR#6 | | | | | | | | | | | |
| | | | pool | | | | | | | | | | | |
| 3 | 72.8 | saNSR.1 | sa.anti- | Jurkat 1 | 17.6 | 29.2 | 125.6 | 169.3 | 551.5 | 964.3 | 1310.5 | 312.9 | 159.0 | 80.5 |
| | | pool + | NSR#5 pool + | | | | | | | | | | | |
| | | 3 pool | sa.anti-NSR#7 | | | | | | | | | | | |
| | | | pool | | | | | | | | | | | |
| 4 | 78.2 | saNSR.1 | sa.anti- | Jurkat 1 | 12.6 | 55.3 | 155.5 | 272.9 | 538.2 | 964.1 | 610.4 | 639.8 | 1041.1 | 787.1 |
| | | pool + | NSR#5 pool + | | | | | | | | | | | |
| | | 4 pool | sa.anti-NSR#8 | | | | | | | | | | | |
| | | | pool | | | | | | | | | | | |
| 5 | 77.1 | saNSR.1 | sa.anti-NSR#5 | Jurkat 2 | 11.5 | 51.0 | 183.5 | 331.2 | 922.5 | 1228.1 | 609.5 | 1210.9 | 221.1 | 126.6 |
| | | | pool | | | | | | | | | | | |
| 6 | 46.2 | saNSR.1 + 2 | sa.anti-NSR#5 | Jurkat 2 | 7.4 | 34.7 | 180.6 | 405.1 | 364.3 | 1560.1 | 410.9 | 1799.2 | 4385.0 | 1007.9 |
| | | | pool + | | | | | | | | | | | |
| | | | sa.anti-NSR#6 | | | | | | | | | | | |
| | | | pool | | | | | | | | | | | |
| 7 | 45.2 | saNSR.1 + 3 | sa.anti- | Jurkat 2 | 20.9 | 30.6 | 107.6 | 234.1 | 378.8 | 1581.6 | 771.5 | 310.6 | 276.1 | 142.5 |
| | | | NSR#5 pool + | | | | | | | | | | | |
| | | | sa.anti-NSR#7 | | | | | | | | | | | |
| | | | pool | | | | | | | | | | | |
| 8 | 81.7 | saNSR.1 + 4 | sa.anti- | Jurkat 2 | 9.7 | 71.9 | 182.1 | 249.9 | 820.5 | 1059.7 | 886.2 | 933.7 | 1192.8 | 1075.4 |
| | | | NSR#5 pool + | | | | | | | | | | | |
| | | | sa.anti-NSR#8 | | | | | | | | | | | |
| | | | pool | | | | | | | | | | | |
| 9 | 72.5 | saNSR.1 | sa.anti-NSR#5 | K562 | 0.6 | 36.2 | 143.9 | 219.3 | 769.3 | 930.1 | 545.8 | 1275.9 | 152.3 | 279.2 |
| | | | pool | | | | | | | | | | | |
| 10 | 69.1 | saNSR.1 + 2 | sa.anti-NSR#5 | K562 | 0.3 | 46.5 | 139.9 | 146.6 | 492.9 | 691.6 | 602.0 | 1562.6 | 3291.7 | 889.2 |
| | | | pool + | | | | | | | | | | | |
| | | | sa.anti-NSR#6 | | | | | | | | | | | |
| | | | pool | | | | | | | | | | | |
| 11 | 73.5 | saNSR.1 + 3 | sa.anti- | K562 | 1.1 | 24.1 | 108.4 | 138.1 | 586.9 | 914.5 | 1480.4 | 481.7 | 150.1 | 224.2 |
| | | | NSR#5 pool + | | | | | | | | | | | |
| | | | sa.anti-NSR#7 | | | | | | | | | | | |
| | | | pool | | | | | | | | | | | |
| 12 | 75.9 | saNSR.1 + 4 | sa.anti- | K562 | | | | | | | | | | |
| | | | NSR#5 pool + | | | | | | | | | | | |
| | | | sa.anti-NSR#8 | | | | | | | | | | | |
| | | | pool | | | | | | | | | | | |
| 13 | 43.6 | Y4R-NSR | Y4F-N9 | Jurkat 1 | 6.7 | 126.1 | 1830.6 | 3675.6 | 874.0 | 5637.9 | 904.2 | 293.6 | 1437.9 | 1644.5 |
| 14 | 59.0 | Y4-N7 | Y4F-N9 | Jurkat 1 | 7.0 | 562.9 | 5317.4 | 19201.8 | 2489.9 | 23678.1 | 2463.8 | 355.5 | 1243.7 | 1751.5 |
| 15 | 47.5 | Y4R-NSR | Y4F-N9 | Jurkat 2 | 7.7 | 253.5 | 2669.7 | 6898.6 | 1716.2 | 7254.4 | 1396.9 | 457.5 | 2184.7 | 3482.8 |
| 16 | 59.0 | Y4-N7 | Y4F-N9 | Jurkat 2 | 7.1 | 286.6 | 2948.3 | 11437.4 | 1977.7 | 18794.7 | 1857.7 | 282.7 | 1119.2 | 1528.5 |
| 17 | 50.2 | Y4R-NSR | Y4F-N9 | K562 | 0.4 | 139.2 | 1939.0 | 3940.1 | 939.7 | 4801.4 | 614.6 | 420.6 | 1423.4 | 3997.5 |
| 18 | 54.1 | Y4-N7 | Y4F-N9 | K562 | 0.5 | 517.5 | 4292.3 | 14486.7 | 1673.4 | 15459.0 | 1590.5 | 285.6 | 849.2 | 1870.3 |
| 19 | 44.8 | N8 | None-RT only, | Jurkat 1 | 0.4 | 648.0 | 3626.8 | 341.3 | 1778.6 | 7321.5 | 1183.5 | 299.8 | 323.8 | 95.4 |
| | | | no second | | | | | | | | | | | |
| | | | strand | | | | | | | | | | | |
| | | | synthesis | | | | | | | | | | | |
| 20 | 46.5 | N8 | None-RT only, | Jurkat 2 | 0.4 | 758.9 | 4521.8 | 513.6 | 2302.5 | 9776.5 | 1396.9 | 321.6 | 327.5 | 104.3 |
| | | | no second | | | | | | | | | | | |
| | | | strand | | | | | | | | | | | |
| | | | synthesis | | | | | | | | | | | |
| 21 | 44.6 | N8 | None-RT only, | K562 | 0.0 | 734.6 | 3460.3 | 496.4 | 2191.6 | 8023.3 | 1344.0 | 286.5 | 298.8 | 139.1 |
| | | | no second | | | | | | | | | | | |
| | | | strand | | | | | | | | | | | |
| | | | synthesis | | | | | | | | | | | |

¹= FAM 10
²= FAM1000
³= Hs99999901

FIG. 4A graphically illustrates the gene-specific polyA content of cDNA amplified using various NSR primers during first strand synthesis and anti-NSR primers or random primers during second strand synthesis as determined using a set of representative gene-specific assays for PPIA, SRP14, STMN1, TRIM63, ACTB, DBN1, EIF3S3, GAPDH, and NUCB2.

Relative abundance of the polyA content shown in FIG. 4A was calculated by first combining the input adjusted raw abundance values of individual rRNA assays by transcript. The collapsed rRNA transcript abundance values were normalized to NUCB2 gene levels measured within each sample preparation such that gene content was equal to 1.0. The rRNA/gene ratios calculated for amplified samples were then normalized to that obtained for the unamplified control (N8) such that N8 was equal to 100 for each rRNA transcript. Therefore, the N8 was used as the standard value for the abundance level of each gene.

With regard to the figure legend for FIG. 4A and FIG. 4B, with reference to TABLE 2 and TABLE 3, saNSR.1 refers to cDNA amplified using NSR#1 primer pool in the first strand synthesis and anti-NSR#5 primer pool in the second strand synthesis (i.e., depleted for rRNA, mt-rRNA and globin in first and second strand synthesis). saNSR.1+2 refers to cDNA amplified using NSR#1+#2 primer pools in the first strand synthesis and anti-NSR#5+#6 primer pools in the second strand synthesis (i.e., depleted for rRNA and globin, but not depleted for mt-rRNA in both first and second strand synthesis). saNSR.1+3 refers to cDNA amplified using NSR#1+#3 primer pools in the first strand synthesis and anti-NSR #5+#7 primer pools in the second strand synthesis (i.e., depleted for rRNA and mt-rRNA, but not depleted for globin in both first and second strand synthesis). saNSR.1+4 refers to cDNA amplified using NSR#1+#4 primer pools in the first strand synthesis and anti-NSR#5+#8 primer pools in the second strand synthesis (i.e., depleted for rRNA, but not depleted for mt-rRNA and globin in both first and second strand synthesis). Y4R-NSR refers to cDNA amplified using NSR primers including the core set of 6-mer NSR oligos with no perfect match to globin (alpha or beta), no perfect match to rRNA (18S, 28S). for first strand synthesis, and random 9-mer primers for the second strand synthesis (i.e., depleted for globin and rRNA, but not depleted for mt-rRNA in the first strand synthesis, but not depleted for any sequences in the second strand synthesis). Y4-N7 refers to cDNA amplified using random 7-mer primers during first and second strand synthesis. Finally, N8 refers to first strand synthesis using random 8mers (no second strand synthesis).

As shown in FIG. 4A, the NSR priming for first strand synthesis amplified gene-specific transcripts at least as efficiently as random primers, with the exception of the gene TRIM63.

FIG. 4B graphically illustrates the relative abundance level of non-polyadenylated RNA transcripts in cDNA amplified from Jurkat-1 and Jurkat-2 total RNA using various NSR primers during first strand cDNA synthesis. As shown in FIG. 4B, gene specific content in the cDNA amplified using NSR and anti-NSR primers is enriched as the rRNA and mt-rRNA content is decreased. This demonstrates that NSR-dependent rRNA depletion is not a general effect, but rather is specific to the transcripts targeted for removal. These results also demonstrate that both polyA minus and polyA plus transcripts are reproducibly amplified using NSR-PCR.

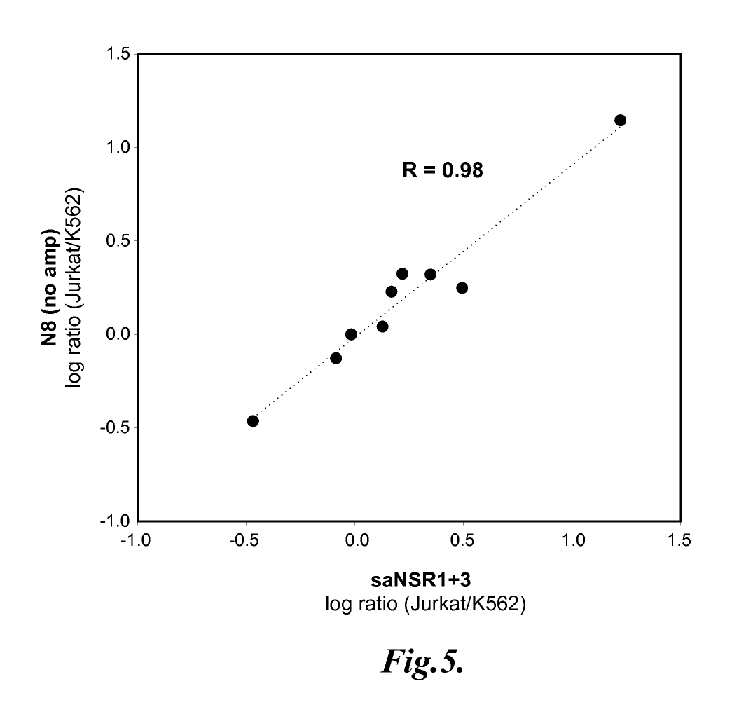

FIG. 5 graphically illustrates the log ratio of Jurkat/K562 mRNA expression data measured in cDNA generated using the primer pool NSR#1+#3 (x-axis) versus the log ratio of Jurkat/K562 mRNA expression data measured in cDNA generated using the random primer pool N8 (no amplification). This result shows that the relative abundance of messenger RNA in different samples is preserved through NSR priming and PCR amplification.

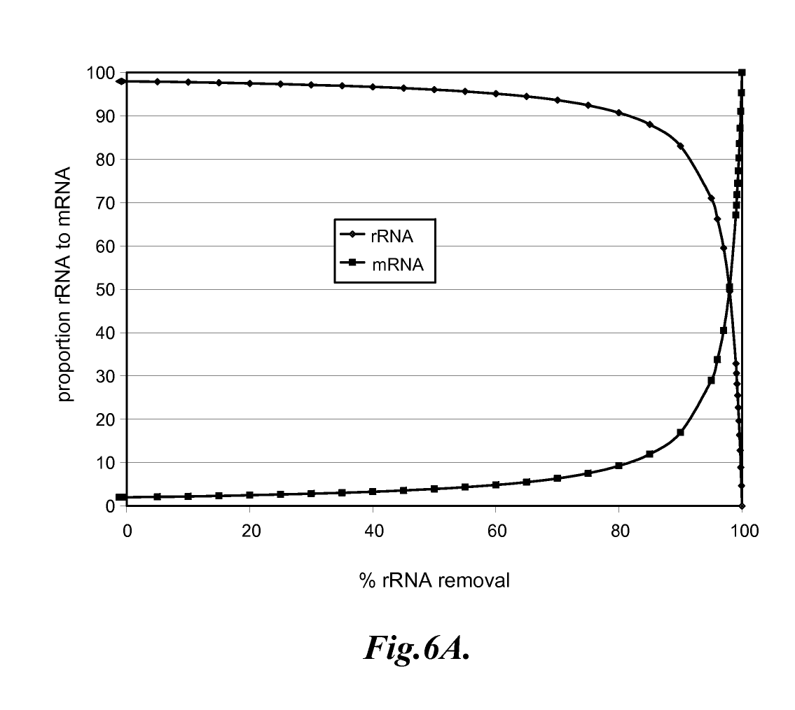

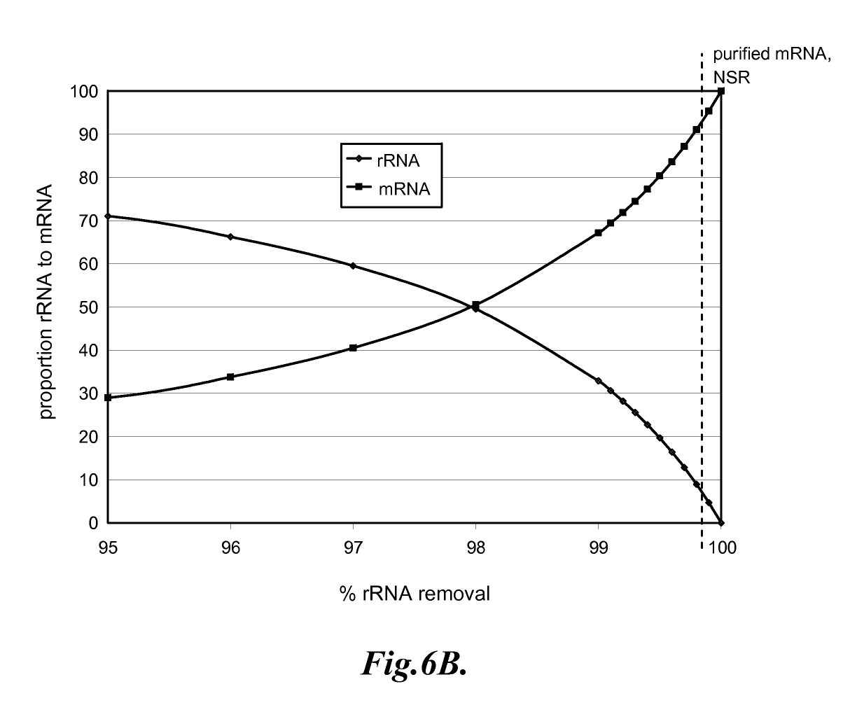

FIG. 6A graphically illustrates the proportion of rRNA to mRNA in total RNA that is typically obtained after polyA purification using conventional methods. As shown in FIG. 6A, prior to polyA purification, total RNA isolated from a mammalian cell includes approximately 98% rRNA and approximately 2% mRNA and other (non-polyA RNA). As shown, even after 95% removal of rRNA from total RNA using polyA purification, the remaining RNA consists of a mixture of about 50% rRNA and 50% mRNA.

FIG. 6B graphically illustrates the proportion of rRNA to mRNA in a cDNA sample prepared using NSR primers during first strand cDNA synthesis and anti-NSR primers during second strand cDNA synthesis. As shown in FIG. 6B the use of NSR primers and anti-NSR primers to generate cDNA from total RNA is effective to remove 99.9% rRNA (including nuclear and mitochondrial rRNA), resulting in a cDNA population enriched for greater than 95% mRNA. This is a very significant result for several reasons. First, the use of polyA purification or strategies that rely on primer binding to the polyA tail of mRNA exclude non-polyA containing RNA molecules such as, for example, miRNA and other molecules of interest, and therefore exclude nucleic acid molecules that contribute to the richness of the transcriptome. In contrast, the methods of the present invention that include the use of NSR primers and anti-NSR primers during cDNA synthesis do not require polyA selection and therefore preserve the richness of the transcriptome. Second, the use of NSR and anti-NSR primers during cDNA synthesis is effective to generate cDNA with removal of 99.9% rRNA, resulting in cDNA with less than 10% rRNA contamination, as shown in FIG. 6B. This is in contrast to polyA purified mRNA and cDNA synthesis using random primers that only removes 98% rRNA, resulting in cDNA with approximately 50% mRNA and 50% rRNA contamination, as shown in FIG. 6A.

### CONCLUSION

These results demonstrate that the NSR #1+#3 primer pool (SEQ ID NOS:1-749) and anti-NSR primer pool (SEQ ID NOS:750-1498) works remarkably well for first strand and second strand cDNA synthesis, respectively, resulting in a double-stranded cDNA product that is substantially enriched for target genes (including poly-adenylated and non-polyadenylated RNA) with a low level (less than 10%) of unwanted rRNA and mt-rRNA.

### Example 4

This Example shows that the use of the 749 NSR-6mers (SEQ ID NOS:1-749) (each has a spacer N and the PBS#1 (SEQ ID NO:1499) covalently attached at the 5′ end) for first strand cDNA synthesis and the use of the 749 anti-NSR-6mers (SEQ ID NOS:750-1498) (that each have a spacer N and the PBS#2 (SEQ ID NO:1500) covalently attached at the 5′ end) prime the amplification of a substantial fraction of the transcriptome (both polyA+ and polyA−) and do not prime unwanted non-target sequences present in total RNA, as determined by sequence analysis of the amplified cDNA.

Methods:

cDNA was generated using 749 NSR-6mers (SEQ ID NOS:1-749) (each has a spacer N and the PBS#1 (SEQ ID NO:1499) covalently attached at the 5′ end) for first strand cDNA synthesis and the use of the 749 anti-NSR-6mers (SEQ ID NOS:750-1498) (each has a spacer N and the PBS#2 (SEQ ID NO:1500) covalently attached at the 5′ end), with the various primer pools shown in TABLE 8, using the methods described in Example 2.

TABLE 8
Protocols Used to Selectively Amplify cDNA

| Protocol | | Second Strand | | |
|---|---|---|---|---|
| Reference | First Strand cDNA | cDNA Synthesis | | Number |
| Number | Primers | Primers | Comments | of Exp |
| NSR-V1 | NSR primers | N7 random | Reaction conditions: | n = 170 |
| | (no perfect match to | | RT run with Y4 primer tails | |
| | rRNA, no globin, + | | (SEQ ID NO: 1504) high dNTP | |
| | mt rRNA) | | (25 mM), 2 hrs at 40° C., 30 min | |
| | | | RNAsH treatment and a | |
| | | | 95° C. denaturation step | |
| NSR-V2 | NSR primers (no | N7 random | Reaction conditions: primers | n = 130 |
| | perfect match to | | and conditions the same as | |
| | rRNA, no globin, + | | above for NSR-V1 except | |
| | mt rRNA) | | RNAse treatment for 10 | |
| | | | minutes and 95° C. denaturation | |
| | | | step was eliminated | |
| NSR-V3 | NSR primers (no | N7 random | Reaction Conditions: primers | n = 187 |
| | perfect match to | | and conditions the same as | |
| | rRNA, no globin, + | | above for NSR-V2 except | |
| | mt rRNA) | | RNAse treatment was | |
| | | | eliminated | |
| NSR-V4 | NSR primers (no | anti-NSR | Reaction Conditions: primers | n = 187 |
| | perfect match to | (SEQ ID | (SEQ ID NO: 1501) were used; | |
| | rRNA, no mt-RNA + | NOS: 750-1499) | reaction conditions as | |
| | globin) | | described in Example 2. | |
| | (SEQ ID NOS: | | | |
| | 1-749) | | | |
| NSR-V5 | NSR | anti-NSR | Reaction conditions: primers | n = 187 |
| | (no perfect match to | (SEQ ID | and conditions-same as | |
| | rRNA, no mt-RNA + | NOS: 750-1499) | NSR-V4 with additional | |
| | globin) | | cleanup step between 1st and | |
| | (SEQ ID NOS: | | 2nd strand synthesis | |
| | 1-749) | | | |
| N7 | N7 Random | N7 Random | Reaction Conditions: same | n = 171 |
| | | | conditions as NSR-V5 with | |
| | | | random N7 primers | |

The cDNA products were PCR amplified and column purified as described in Example 2. The column-purified PCR products were then cloned into TOPO vectors using the pCR-XL TOPO kit (Invitrogen). The TOPO ligation reaction was carried out with 1 μl PCR product, 4 μl water and 1 μl of vector. Chemically competent TOP10 One Shot cells (Invitrogen) were transformed and plated onto LB+Kan (50 μg/mL) and grown overnight at 37° C. Colonies were screened for inserts using PCR amplification. It was determined by 2% agarose gel analysis that all clones had inserts of at least 100 by (data not shown).

The clones were then used as templates for DNA sequence analysis. Resulting sequences were run against a public database for determining homology to rRNA species and the genome.

Results:

TABLE 9 provides the results of sequence analysis of the PCR products generated from cDNA synthesized using the various primer pools shown in TABLE 8.

TABLE 9
Results of DNA Sequence Analysis of aDNA Generated From
Selectively Amplified cDNA

| Primers | rRNA | mt-RNA | | |
|---|---|---|---|---|
| Used | (% of Total) | (% of Total) | Gene-Specific | |
| for cDNA | (18S or 28S | (12S or 16S | RNA¹ | Other² |
| Synthesis | rRNA) | rRNA) | (% of Total) | (% of Total) |
| N7 | 77.2 | 8.2 | 13.5 | 1.2 |
| NSR-V1 | 44.7 | 19.4 | 28.8 | 7.1 |
| NSR-V2 | 17.0 | 20.0 | 51.0 | 12.0 |
| NSR-V3 | 2.0 | 17.0 | 64.0 | 17.0 |
| NSR-V4 | 10.7 | 5.3 | 67.4 | 16.6 |
| NSR-V5 | 3.7 | 3.2 | 78.6 | 14.4 |

¹= determined to overlap with any known gene or mRNA including exon, intron, and UTR regions as determined by sequence alignment with public databases.
²= determined to overlap with repeat elements or alignment to intergenic regions as determined by sequence alignment with public databases.

### CONCLUSION

These results demonstrate that aDNA (PCR products) amplified from double-stranded cDNA templates generated using the NSR 6-mers (SEQ ID NOS:1-749), and anti-NSR6-mers (SEQ ID NOS:750-1498) as described in Example 2, preserved the enrichment of target genes relative to nuclear ribosomal RNA and mitochondrial ribosomal RNA.

### Example 5

This Example describes methods that are useful to label the aDNA (PCR products) for subsequent use in gene expression monitoring applications.

1. Direct Chemical Coupling of Fluorescent Label to the PCR Product.

Cy3 and Cy5 direct label kits were obtained from Minis (Madison, Wis., kit MIR Product Numbers 3625 and 3725).

10 μg of PCR product (aDNA), obtained as described in Example 2, was incubated with labeling reagent as described by the manufacturer. The labeling reagents covalently attach Cy3 or Cy5 to the nucleic acid sample, which can then be used in almost any molecular biology application, such as gene expression monitoring. The labeled aDNA was then purified and its fluorescence was measured relative to the starting label.

Results:

Four aDNA samples were labeled as described above and fluorescence was measured. A range of 0.9 to 1.5% of retained label was observed across the four labeled aDNA samples (otherwise referred to as a labeling efficiency of 0.9 to 1.5%). These results fall within the 1% to 3% labeling efficiency typically observed for aaUTP labeled, in vitro translated, amplified RNA.

2. Incorporation of Aminoallyl Modified dUTP (aadUTP) During PCR with an aDNA Template Using One Primer (Forward or Reverse) to Yield aa-Labeled, Single-Stranded aDNA.

Methods:

1 μg of the aDNA PCR product, generated using the NSR and anti-NSR primer pool as described in Example 2, is added to a PCR reaction mix as follows:
100 to 1000 μM aadUTP+dCTP+cATP+dGTP+dUTP (the optimal balance of aadUTP to dUTP may be empirically determined using routine experimentation)
4 mM MgCl₂
400-1000 nM of only the forward or reverse primer, but not both.

PCR Reaction: 5 to 20 cycles of PCR (94° C. 30 seconds, 60° C. 30 seconds, 72° C. 30 seconds), during which time only one strand of the double-stranded PCR template is synthesized. Each cycle of PCR is expected to produce one copy of the aa-labeled, single-stranded aDNA. This PCR product is then purified and a Cy3 or Cy5 label is incorporated by standard chemical coupling.

3. Incorporation of Aminoallyl Modified dUTP (aadUTP) During PCR with an aDNA Template Using Forward and Reverse Primers to Yield aa-Labeled, Double-Stranded aDNA.

Methods:

1 μg of the aDNA PCR product generated using the NSR7 primer pool as described in Example 11 is added to a PCR reaction mix as follows:
100 to 1000 μM aadUTP+dCTP+cATP+dGTP+dUTP (the optimal balance of aadUTP to dUTP may be empirically determined using routine experimentation)
4 mM MgCl₂
400-1000 nM of the forward and reverse primer (e.g., Forward: SEQ ID NO:1501; or Reverse: SEQ ID NO:1502)

PCR Reaction: 5 to 20 cycles of PCR (94° C. 30 seconds, 60° C. 30 seconds, 72° C. 30 seconds), during which time both strands of the double-stranded PCR template are synthesized. The double-stranded, aa-labeled aDNA PCR product is then purified and a Cy3 or Cy5 label is incorporated by standard chemical coupling.

### Example 6

This Example describes the use of a hybrid RNA/DNA primer covalently linked to NSR-6mers to generate amplified nucleic acid templates useful for generating single-stranded DNA molecules for gene expression analysis.

Rationale: In one embodiment of the selective amplification methods of the invention, the defined sequence portion (e.g., PBS#1) of a first oligonucleotide population for first strand cDNA synthesis, and/or the defined sequence portion (e.g., PBS#2) of a second oligonucleotide population for second strand cDNA synthesis comprises an RNA portion to generate an amplified nucleic acid template suitable for generating multiple copies of DNA products using strand displacement, as described in U.S. Pat. No. 6,946,251, hereby incorporated by reference. A hybrid NSR primer (PBS#1(RNA/DNA)/NSR) may be used to synthesize first strand cDNA, thereby generating products suitable for use as templates for synthesis of single-stranded DNA having a sequence complementary to template RNA. Alternatively, an RNA/DNA hybrid primer tail may be added after second strand synthesis, as described in more detail below.

One advantage provided by this method is the ability to generate a plurality of single-stranded amplification products of the original cDNA sequence, and not the amplification of the product of the amplification itself.

Methods:

1. RNA:DNA hybrid NSR for First Strand cDNA Synthesis:

In some embodiments, the population of NSR primers for use in first strand cDNA synthesis (SEQ ID NOS:1-749) may further comprise a 5′ primer binding sequence (RNA), such as hybrid PBS#1:

Hybrid PBS#1(RNA)

| | |
|---|---|
| 5′ GACGGAUGCGGUCU 3′ | (SEQ ID NO: 1557) |

covalently attached at the 5′ end of the NSR
primers.

Resulting in a population of RNA:DNA hybrid oligonucleotides having an RNA defined sequence portion located 5′ to the DNA hybridizing portion with the following configuration:

5′ hybrid PBS#1(RNA) (SEQ ID NO: 1557) +
NSR6-mer (DNA) (SEQ ID NOS: 1-749) 3′

In another embodiment, a population of oligonucleotides may be generated wherein each NSR6-mer optionally includes at least one DNA spacer nucleotide (N) (where each N=A, G, C, or T) where (N) is located between the 5′ hybrid PBS#1 (RNA) and the NSR6mer (DNA). The spacer region may comprise from one nucleotide up to ten or more nucleotides (N=1 to 10), resulting in a population of oligonucleotides having the following configuration:

5′ Hybrid PBS#1(RNA) (SEQ ID NO: 1557) + (N₁₋₁₀)
(DNA) + NSR6-mer (SEQ ID NOS: 1-749) (DNA)3′

The process of preparing the first strand cDNA is carried out essentially as described in Example 2, with the substitution of the hybrid PBS#1 (SEQ ID NO:1557) (RNA) for the PBS#1 (SEQ ID NO:1499) (DNA), with the use of an RNAseH-reverse transcriptase and without the addition of RNAseH prior to second strand cDNA synthesis, to generate a double-stranded substrate for amplification of single-stranded DNA products

The substrate for single stranded amplification preferably consists of a double stranded template with the first strand consisting of an RNA/DNA hybrid molecule and the second strand consisting of all DNA. In order to construct this double-stranded template, second strand synthesis is carried out using an RNAseH-reverse transcriptase. Alternatively, the second strand synthesis may be carried out using Klenow followed by a polished step with RNAseH-reverse transcriptase, since Klenow will not use RNA as a template.

Second strand cDNA synthesis may be carried out using either random primers, or using anti-NSR primers. The use of the RNA hybrid/NSR primer population during first strand cDNA synthesis results in the incorporation of a unique sequence of the RNA portion of the hybrid primer into the synthesized single-stranded cDNA product.

Single-stranded DNA amplification products that are identical to the target RNA sequence may then be generated from the double-stranded template described above by denaturing and RNAseH treating the denatured substrate to remove the RNA portion of the substrate, and adding a hybrid RNA/DNA single-stranded amplification primer, e.g., 5′ GACGGAUGCGGTGT 3′ (SEQ ID NO:1558), where the 5′ portion of the primer consists of at least eleven RNA nucleotides (underlined) that hybridize to a predetermined sequence on the first strand cDNA and the 3′ portion consists of at least three DNA nucleotides to the substrate in the presence of a highly processive strand displacing DNA polymerase, such as, for example, phi29.

In alternative embodiment, the substrate for single-stranded DNA amplification may be prepared by preparing first strand cDNA synthesis using DNA primers (e.g., NSR or random primers), followed by second strand synthesis with Klenow also using DNA primers (e.g., anti-NSR or random primers). The double-stranded DNA template is then modified to produce a substrate for single-stranded DNA amplification by denaturing and annealing an RNA/DNA hybrid oligonucleotide that hybridizes to the second strand cDNA and extending the hybrid RNA/DNA oligonucleotide with Reverse Transcriptase, to generate a double stranded template with one strand consisting of an RNA/DNA hybrid molecule and the other strand consisting of all DNA.

Single stranded DNA amplification products that are complementary to the target RNA sequence may then be generated from the double-stranded substrate by denaturing and RNAseH treating the denatured substrate to remove the RNA portion of the substrate. A hybrid RNA/DNA single-stranded amplification primer is then annealed to the second strand, wherein the 5′ portion of the hybrid primer consists of at least eleven RNA nucleotides that hybridize to a pre-determined sequence on the second strand cDNA and the 3′ portion of the hybrid primer consists of at least three DNA nucleotides. A highly processive strand displacing DNA polymerase, such as, for example, phi29. is then used to generate single-stranded DNA products.

### Example 7

This Example describes the robust detection of poly A+ and poly A− transcripts in cDNA amplified from total RNA using NSR primers.

Rationale:

The whole transcriptome, that is, the entire collection of RNA molecules present within cells and tissues at a given instant in time, carries a rich signature of the biological status of the sample at the moment the RNA was collected. However, the biochemical reality of total RNA is that an overwhelming majority of it codes for structural subunits of cytoplasmic and mitochondrial ribosomes, which provide relatively little information on cellular activity. Consequently, molecular techniques that enrich for more informative low copy transcripts have been developed for large-scale transcriptional studies, such as the exploitation of 3′ polyadenylation sequences as an affinity tag for non-ribosomal RNA. Targeted sequencing of polyA+ RNA transcripts has provided a rich foundation of cDNA fragments that form the basis of current gene models (see e.g., Hsu F. et al., Bioinformatics 22:1036-1046 (2006)). Priming of cDNA synthesis from polyA sequences has also been used for the most commonly practiced, genome-wide RNA profiling methods.

Although these methods have been very successful for analysis of messenger RNA expression, methods that strictly focus on polyA+ transcripts present an incomplete view of global transcriptional activity. PolyA priming often fails to capture information distal to 3′ polyA sites, such as alternative splicing events and alternative transcriptional start sites. Conventional methods also fail to monitor expression of non-poly-adenylated transcripts including those that encode protein subunits of histone deacetylase and many non-coding RNAs. Although alternative methods have been developed to specifically target many of these RNA sub-populations (Johnson J. M. et al., Science 302:2141-2144 (2003); Shiraki T. et al., PNAS 100:15776-15781 (2003); Vitali P. et al., Nucleic Acids Res. 31:6543-6551 (2003)), only a few studies have attempted to monitor all transcriptional events in parallel. The most comprehensive analysis of whole transcriptome content has been carried out using genome tiling arrays (Cheng J. et al., Science 308:1149-1154 (2005); Kapranov P. et al., Science 316:1484-1488 (2007)). However, the complexity of these experiments and the need for subsequent validation by complementary methods has limited the use of tiling arrays for routine whole transcriptome profiling applications. Recent advances in DNA sequencing present an opportunity for new approaches to expression analysis, allowing both the quantitative assessment of RNA abundance and experimentally-verified transcript discovery on a single platform (Mortazavi A. et al., Nat. Methods 5:621-628 (2008)). Therefore, there is a need for a method that provides an unbiased survey of both known and novel transcripts that can utilize high-throughput profiling of numerous samples.

Methods:

Overview:

In accordance with the foregoing, the inventors have developed a sample preparation procedure that relies on the “not-so-random” (“NSR”) priming libraries in which all hexamers with perfect matches to ribosomal RNA (rRNA) sequences have been removed. For NSR selective priming to be useful as a whole transcriptome profiling technology, it must faithfully detect non-ribosomal RNA transcripts. To test the performance of NSR-priming, a whole transcriptome cDNA library was constructed. Antisense NSR hexamers (“NSR” primers) were synthesized to prime first strand synthesis, with a universal tail sequence to facilitate PCR amplification and downstream sequencing using the Illumina 1G Genome Analyzer. A second set of tailed NSR hexamers complementary to the first set of NSR primers (“anti-NSR” primers) was generated to prime 2nd strand synthesis. The unique tail sequences used for first and second strand NSR primers enabled the preservation of strand orientation during amplification and sequencing. For this study, all sequencing reads were oriented in a 3′ to 5′ direction with respect to the template RNA, although opposite strand reads can be easily generated by modifying the universal PCR amplification primers.

To evaluate whole transcriptome content in NSR-primed libraries, a survey was conducted of NSR-primed cDNA libraries generated from the RNA isolated from whole brain and RNA isolated from the Universal Human Reference (UHR) cell line (Stratagene) by sequencing, as described below.

Oligonucleotides Used to Generate Libraries:

A first population of NSR-6mer primers 5′ (SEQ ID NO:1499) covalently attached to each of (SEQ ID NOS:1-749) was used for amplification of the first strand and a second population of anti-NSR-6mer primers (SEQ ID NO:1500) covalently attached to each of (SEQ ID NOS:750-1498) for use in second strand cDNA synthesis, as described in Example 1. Oligos were desalted and resuspended in water at 100 uM before pooling.

A collection of random hexamers were also synthesized with the tail sequences SEQ ID NO:1499 and SEQ ID NO:1500 for generation of control libraries.

Library Generation:

Overview: NSR-priming selectively captures the non-ribosomal RNA fraction including poly A+ and poly A− transcripts. Two rounds of NSR priming selectivity were applied during library construction. First, NSR oligonucleotides (antisense) initiate reverse transcription at not-so-random template sites. Following ribonuclease treatment to remove the RNA template, anti-NSR oligonucleotides (sense) anneal to single-stranded cDNA at not-so-random template sites and direct Klenow-mediated second strand synthesis. PCR amplification with asymmetric forward and reverse primers preserves strand orientation and adds terminal sites for downstream end sequencing. Antisense tag sequencing is then carried out from the 3′ end of cDNA fragments using a portion of the forward amplification primer. Pairwise alignments are then used to map the reverse complements of tag sequences to the human genome.

Methods:

Total RNA from whole brain was obtained from the FirstChoice® Human Total RNA Survey Panel (Ambion, Inc.). Universal Human Reference (UHR) cell line RNA was purchased from Stratagene Corp. Total RNA was converted into cDNA using Superscript™ III reverse transcription kit (Invitrogen Corp). Second strand synthesis was carried out with 3′-5′ exo-Klenow Fragment (New England Biolabs Inc.). DNA was amplified using Expand High Fidelity^{PLUS} PCR System (Roche Diagnostics Corp.).

For NSR primed cDNA synthesis, 2 μl of 100 μM NSR primer mix (SEQ ID NO:1499 plus SEQ ID NOS:1-749) was combined with 1 μl template RNA and 7 μl of water in a PCR-strip-cap tube (Genesee Scientific Corp.). The primer-template mix was heated at 65° C. for 5 minutes and snap-chilled on ice before adding 10 μl of high dNTP reverse transcriptase master mix (3 μl of water, 4 μl of 5× buffer, 1 μL of 100 mM DTT, 1 μl of 40 mM dNTPs and 1.0 μl of SuperScript™ III enzyme). The 20 μl reverse transcriptase reaction was incubated at 45° C. for 30 minutes, 70° C. for 15 minutes and cooled to 4° C. RNA template was removed by adding 1 μl of RNAseH (Invitrogen Corp.) and incubated at 37° C. for 20 minutes, 75° C. for 15 minutes and cooled to 4° C. DNA was subsequently purified using the QIAquick® PCR purification kit and eluted from spin columns with 30 μl elution buffer (Qiagen, Inc. USA).

For second strand synthesis, 25 μl of purified cDNA was added to 65 μl Klenow master mix (46 μl of water, 10 μl of 10×NEBuffer 2, 5 μl of 10 mM dNTPs, 4 μl of 5 units/μL exo-Klenow Fragment, New England Biolabs, Inc.) and 10 μL of 100 μM anti-NSR primer mix (SEQ ID NO:1500 plus SEQ ID NOS:750-1498). The 100 μl reaction was incubated at 37° C. for 30 minutes and cooled to 4° C. DNA was purified using QIAquick spin columns and eluted with 30 μl elution buffer (Qiagen, Inc. USA). For PCR amplification, 25 μL of purified second strand synthesis reaction was combined with 75 μL of PCR master mix (19 μl of water, 20 μl of 5× Buffer 2, 10 μl of 25 mM MgCl₂, 5 ul of 10 mM dNTPs, 10 μl of 10 μM forward primer, 10 μL of 10 μM reverse primer, 1 μL of ExpandPLUS enzyme, Roche Diagnostics Corp.).

Forward PCR primer:
(SEQ ID NO: 1559)
(5′ATGATACGGCGACCACCGACACTCTTTCCCTACACGACGCTCTT
CCGATCTCT3′)
Reverse PCR primer:
(SEQ ID NO: 1560)
(5′CAAGCAGAAGACGGCATACGAGCTCTTCCGATCTGA3′)

Samples were denatured for 2 minutes at 94° C. and followed by 2 cycles of 94° C. for 10 seconds, 40° C. for 2 minutes, 72° C. for 1 minute, 8 cycles of 94° C. for 10 seconds, 60° C. for 30 seconds, 72° C. for 1 minute, 15 cycles of 94° C. for 15 seconds, 60° C. for 30 seconds, 72° C. for 1 minute with an additional 10 seconds added at each cycle; and 72° C. for 5 minutes to polish ends before cooling to 4° C. Double-stranded DNA was purified using QIAquick spin columns

A control library was generated using the same methods with the use of random primers, expect for the concentration of dNTPs was 0.5 mM (rather than 2.0 mM) in the final reverse transcription reaction. The random primed control library was amplified using the PCR primers SEQ ID NO:1559 and SEQ ID NO:1560.

Quantitative PCR:

Individual rRNA and mRNA transcripts were quantified by qPCR using TaqMan® Gene Expression Assays (Applied Biosystems). qPCR Assays were carried out using the reagents shown below in TABLE 10.

TABLE 10
Primers for qPCR Assay

| | | | | FAM |
|---|---|---|---|---|
| | | Forward | Reverse | reporter |
| Target | ABI Assay Probe | Primer | Primer | primer |
| PPIA | Hs99999904_m1 | NR | NR | NR |
| peptidylprolyl | | | | |
| isomerase A | | | | |
| (cyclophilin A) | | | | |
| STMN1 | Hs01027516_g1 | NR | NR | NR |
| stathmin 1/ | | | | |
| oncoprotein 18 | | | | |
| EIF3S3 | Hs00186779_m1 | NR | NR | NR |
| eukaryotic | | | | |
| translation | | | | |
| initiation factor 3, | | | | |
| subunit 3 gamma, | | | | |
| 40 kDa | | | | |
| 18s rRNA | Hs99999901_s1 | NR | NR | NR |
| 12S rRNA | custom | SEQ ID | SEQ ID | SEQ ID |
| | | NO: 1532 | NO: 1533 | NO: 1534 |
| 16S rRNA | custom | SEQ ID | SEQ ID | SEQ ID |
| | | NO: 1526 | NO: 1527 | NO: 1528 |
| 28S rRNA | custom | SEQ ID | SEQ ID | SEQ ID |
| | | NO: 1511 | NO: 1512 | NO: 1513 |

Triplicate measurements of diluted library DNA were made for each assay in 10 μl final reaction volumes in a 384-well optical PCR plate using a 7900 HT PCR instrument (Applied Biosystems). Following PCR, the results table was exported to Excel (Microsoft Corp.), standard curves were generated, and quantitative analysis for samples was regressed from the raw data. Abundance levels were then normalized to input cDNA mass.

Results of qPCR Analysis:

Comparison of cDNA libraries generated from whole brain total RNA using either NSR-priming or a nonselective priming control of random sequence, tailed heptamers revealed a significant depletion of rRNA and a concomitant enrichment of target mRNA in NSR-primed libraries. Specifically, a >95% reduction was observed in the abundance of all four of the rRNA transcripts included in the computational filter used for NSR primer design (data not shown).

Sequence and Read Classification:

In order to obtained a detailed view of rRNA depletion in NSR primed libraries, tag sequences were generated as 36 nucleotide antisense reads from NSR-primed (2.6 million) and random-primed (3.8 million) cDNA libraries using the Illumina 1G Genome Analyzer (Illumina, Inc.). To characterize sequence tags, the dinucleotide barcode (CT) at the 5′ end of each read was removed and the reverse complement of bases 2-34 was aligned to several sequence databases using the ELAND mapping program, which allows up to 2 mismatches per 32 nt alignment (Illumina, Inc.).

To generate expression profiles of RefSeq mRNA and non-coding RNA transcripts, each tag sequence was permitted to align to multiple transcripts. Read counts were then converted to expression values by calculating frequency per 1000 nucleotides from transcript length. A sample normalization factor (nf) was applied to adjust for the total number of reads generated from each library. This was derived from the total number of non-ribosomal RNA reads mapping to the genome for each library (brain 1:17.7 million reads, 1.0 nf; brain 2:19.3 million reads, 1.087 nf; UHR:17.6 million reads, 0.995 nf).

For global classification, sequencing reads were first aligned to the non-coding RNA and repeat databases with alignments to multiple reference sequences permitted. The remaining tag sequences were then mapped to the March 2006 hg18 assembly of the human genome sequence (http:genome.ucsd.edu/). Reads mapping to single genomic sites were classified into mRNA, intron and intergenic categories using coordinates defined by UCSC Known Genes (http://genome.ucsc.edu). Sequences that mapped to multiple genomic sequences that did not include repeats or non-coding RNAs made up the “other” category. Ribosomal RNA sequences were obtained from RepeatMasker (http://www.repeatmasker.org/) and Genbank (NC_{—}001807). Non-coding RNA sequences were collected from Sanger RFAM (http://www.sanger.ac.uk/Software/Rfam/), Sanger miRBASE (http://microrna.sanger.ac.uk), snoRNABase (http://www-snorna.biotoul.fr) and RepeatMasker. Repetitive elements were obtained from RepeatMasker.

Results: More than 54 million high quality 32-nucleotide tag sequence reads that aligned to non-rRNA genomic regions were obtained from two independently prepared whole brain libraries and a single UHR library. Seventy-seven percent of these reads mapped to single genomic sites. Among 22,785 model transcripts in the RefSeq mRNA database (Pruitt K. D. et al., Nucleic Acids Res. 33:D501-504 (2005)), over 87% were represented by 10 or more sequence tag reads in at least some of the samples queried, and 69% were represented by 10 or more reads in all three libraries.

TABLE 11
Results of alignment of 32 nucleotide tag sequence reads
from NSR-primed (2.6 million) and random-primed
(3.8 million) libraries.

| | NSR Primed Library | |
|---|---|---|
| | (1st and 2nd strand | Random-primed |
| Target | NSR) | library |
| large subunit rRNA | 10.3% | 47.2% |
| (includes 5S, 5.8S | | |
| and 28S rRNA | | |
| transcripts) | | |
| small subunit rRNA | 0.8% | 18.0% |
| (includes 18S rRNA | | |
| transcript) | | |
| mitochondrial rRNA | 2.2% | 12.6% |
| (includes 12S and | | |
| 16S rRNA) | | |
| non-ribosomal RNA | 86.7% | 22.2% |
| (includes all other | | |
| sequences that | | |
| mapped to one or | | |
| more genomic sites) | | |

As shown above in TABLE 11, only 13% of sequence tags from NSR primed libraries mapped to the human genome corresponded to ribosomal RNA, whereas 78% of random-primed cDNA matched rRNA sequences. These results demonstrate that NSR-priming resulted in a nearly complete depletion of small subunit 18S rRNA and a dramatic reduction in mitochondrial rRNA transcripts. Although the reduction of large subunit rRNA abundance was less efficient than other rRNA transcripts, relatively modest depletion of 28S RNA can have a large impact on final library composition, owing to its high initial molar concentration and transcript length. In addition, over 86% of NSR-primed sequences mapped to non-rRNA genomic regions compared to 22% of random-primed cDNA. Only 5% of all sequence reads from either library did not map to any genomic sequence, indicating that the library construction process generated very little template-independent artifacts. Similar results were observed from NSR-primed and random-primed libraries generated from UHR total RNA, isolated from a diverse mixture of cell lines (data not shown).

In order to detect polyA+ RefSeq mRNA in NSR-primed libraries, quantitative analysis of sequencing alignments within RefSeq transcripts was used to produce sequence-based digital expression profiles. Excellent reproducibility of NSR-primed cDNA amplification was observed between two separate NSR libraries prepared from the same whole brain total RNA, with a log 10 ratio of transcripts represented by at least 10 NSR tag sequences in replicate #1 versus replicate #2 with a correlation coefficient of r=0.997 for n=17,526.

To assess the accuracy of mRNA profiles obtained from NSR libraries, a comparison was made between the NSR-primed brain profile and the UHR expression profile to the “gold-standard” TaqMan® qPCR profile created for the MicroArray Quality Control Study (MAQC Consortium) (Shi L. et al., Nat. Biotechnol. 24:1151-1161 (2006)),

Correlation of gene expression profiles obtained by NSR tag sequencing and TaqMan® quantitative PCR was also assessed. The log 10 ratios of transcript levels in brain and UHR obtained by NSR tag sequencing were plotted against TaqMan® measurements obtained from the MAQC Consortium with a correlation coefficient of r=0.930 for n=609.

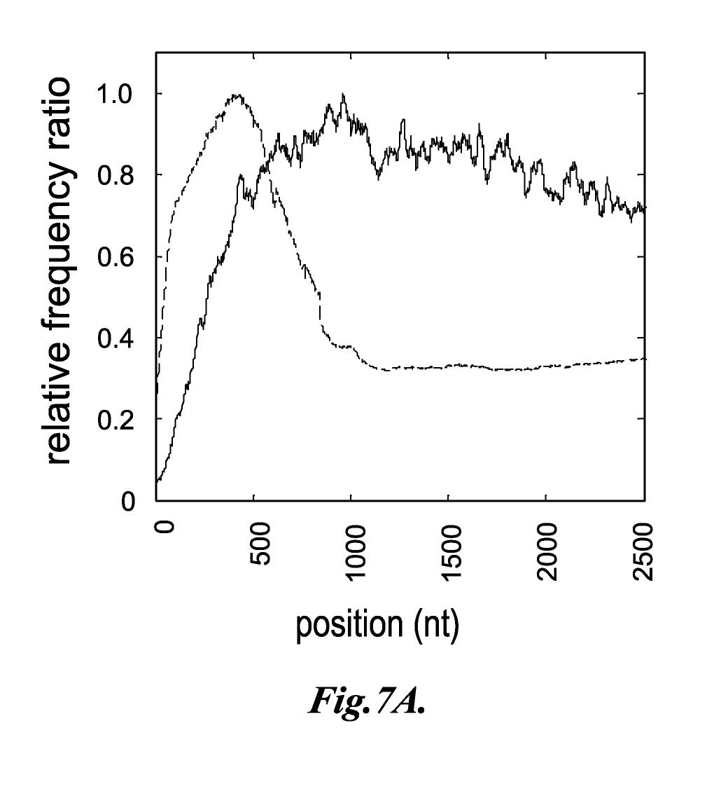

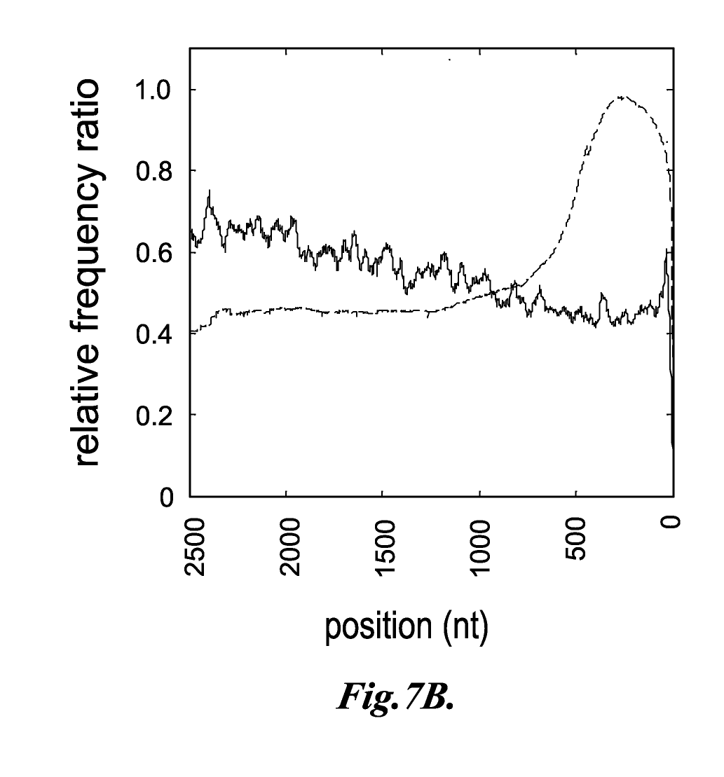

Detection of poly A+ Ref Seq mRNA in NSR-primed libraries was carried out as follows. The positional distribution of NSR tag sequences was examined across transcript lengths. FIG. 7A shows the combined read frequencies for 5,790 transcripts shown at each base position starting from the 5′ termini, with NSR (dotted line) or EST (solid line) cDNAs across long transcripts (≧4 kb). FIG. 7B shows the combined read frequencies for 5,790 transcripts shown at each base position starting from the 3′ termini, with NSR (dotted line) or EST (solid line) cDNAs across long transcripts (≧4 kb). Data shown in FIGS. 7A and 7B were normalized to the maximal value within each dataset. As shown in FIGS. 7A and 7B, NSR-primed cDNA fragments show full-length coverage of large transcripts with higher representation of internal sites than conventional ESTs. This is an important feature of whole transcriptome profiling because the technology preferably captures alternative splicing information. The sequencing coverage exhibited a modest deficit at the extreme 5′ ends of known transcripts owing to the fact that all of the sequencing reads were generated from the 3′ ends of cDNA fragments. This effect may be alleviated if sequencing is directed at both ends of NSR cDNA products. Taken together, these results demonstrate the robustness of NSR-based selective priming as a technology for whole transcriptome expression profiling.

Another requirement of whole transcriptome profiling is that it must effectively capture poly A− transcripts. The representation of poly A− non-coding RNAs in NSR-primed cDNA was determined as follows. Sequence tags from NSR-primed libraries were aligned to a comprehensive database of known poly A− non-coding RNA (ncRNA) sequences. Transcripts representing diverse functional classes were widely detected with a substantial fraction of small nucleolar RNAs (“snoRNAs”) (286/665) and small nuclear RNAs (“snRNAs”) (7/19) present at 5 or more copies in at least one sample. Interestingly, only a small portion of miRNA hairpins and tRNA species were observable at detectable levels. As shown below in TABLE 12, individual transcripts were observed over a broad range of expression levels with members of the snRNA and snoRNA families among the most highly abundant.

TABLE 12
Rank-ordered Expression Levels of non-coding (ncRNA) transcripts
represented by at least two NSR tag sequences in whole brain

| | Log10 | Brain Expression Rank |
|---|---|---|
| ncRNA Transcript/Type | Expression Level | (out of a total of 200) |
| HBII-52 (brain-specific C/D | 6.5 | 1st |
| box snoRNA) | | |
| HBII-85 (brain-specific C/D | 6 | 2nd |
| box snoRNA) | | |
| U2 (snRNA) | 5.8 | 3rd |
| U1 (snRNA) | 5.3 | 5th |
| U3 (snRNA) | 5 | 8th |
| U4 (snRNA) | 4.8 | 10th |
| U13 (snRNA) | 3.7 | 28th |
| U6 (snRNA) | 3.5 | 33rd |
| HBII-436 (brain-specific | 3.4 | 40th |
| C/D box snoRNA) | | |
| HBII-437 (brain-specific | 3.1 | 60th |
| C/D box snoRNA) | | |
| HBII-438A (brain-specific | 2.8 | 85th |
| C/D box snoRNA) | | |
| HBII-13 (brain-specific C/D | 2.7 | 90th |
| box snoRNA) | | |
| U5 (snRNA) | 2.3 | 105th |
| U8 (snRNA) | 2 | 140th |

As shown below in TABLE 13, the NSR-primed libraries containing poly A− transcripts included members of the snRNA and snoRNA families, as well as RNAs corresponding to other well-known transcripts such as 7SK, 7SL and members of the small cajal body-specific RNA family.

TABLE 13
Representation of Major non-coding (ncRNA) Classes in
NSR primed library generated from Whole Brain Total RNA

| polyA-Transcript in | |
|---|---|
| NSR primed library | % of library |
| snoRNA | 60.4% |
| snRNA | 22.1% |
| 7SL | 13.8% |
| 7SK | 4.7% |
| scRNA | 1.3% |
| miRNA | 0.7% |
| tRNA | 0.1% |

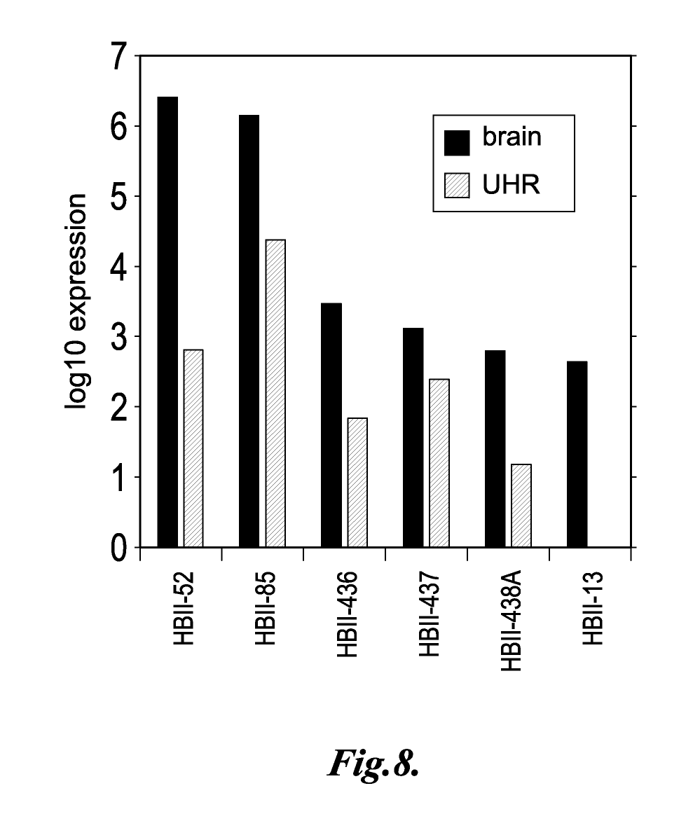

Many transcripts were found to be enriched in the NSR primed library generated from the whole brain total RNA, as compared to the NSR primed library generated from UHR, including the cluster of C/D box snoRNAs located in the q11 region of chromosome 15 that has been implicated in the Prader-Willi neurological syndrome (Cavaile J. et al., J. Biol. Chem. 276:26374-26383 (2001); Cavaile J. et al., PNAS 97:14311-14316 (2000)). FIG. 8 graphically illustrates the enrichment of snoRNAs encoded by the Chromosome 15 Prader-Willi neurological disease locus in whole brain NSR primed library relative to the UHR NSR primed library.

It is interesting to note that a significant proportion of known ncRNA transcripts detected in this study were less than 100 nucleotides in length and were predicted to have extensive secondary structure, thereby also demonstrating that NSR-priming is capable of capturing templates considered problematic to capture using conventional methods.

Global Overview of Transcriptional Activity

The collection of whole transcriptome cDNA sequences generated using NSR priming may be assembled into a global expression map for whole brain and UHR. In order to assemble such a global expression map, all non-ribosomal RNA tag sequences were assigned to one of six non-overlapping categories based on current genome annotations as shown in TABLE 14 below.

TABLE 14
Classification of Whole Transcriptome Expression in
NSR-primed cDNA tags mapping to non-ribosomal
RNA genomic regions

| | NSR-primed | |
|---|---|---|
| | whole Brain | NSR-primed |
| Category | library | UHR library |
| mRNA | 46% | 35% |
| intron | 19% | 30% |
| intergenic | 12% | 13% |
| ncRNA | 4% | 1% |
| repeats | 3% | 6% |
| other | 16% | 15% |

The mRNA, intron and intergenic categories shown above in TABLE 14 were defined by the genomic coordinates of UCSC Known Genes and include only cDNAs that map to unique locations. Sequencing tag reads overlapping any part of a coding exon or UTR were considered mRNA. Sequencing tag reads mapping to multiple genomic sites were binned into the ncRNA, repeats or other categories.

As shown above in TABLE 14, it was determined that tissue and cell line RNA populations exhibited similar overall expression patterns. For example, 65% of tag sequences occurred within the boundaries of known protein-coding genes, whereas only 12-13% of tag sequences mapped to intergenic regions, which is considerably lower than previously reported (Cheng J. et al., Science 308:1149-1154 (2005)). The fraction of cDNAs corresponding to pseudogenes and other redundant sequences, such as motifs shared within gene families (the “other” category in TABLE 14), was also similar in both samples. However, the representation of some categories was notably different in whole brain and UHR. Although intronic expression was substantial in both RNA populations, transcriptional activity in introns was 60% higher in UHR than in whole brain. Expression of repetitive elements was also higher in UHR than in whole brain. In contrast, the cumulative abundance of known ncRNAs was 4-fold higher in brain than UHR. While not wishing to be bound by any particular theory, these results may reflect general differences in splicing activity between cell lines and tissues. Alternatively, these findings may indicate that transcription is generally more pervasive in cell lines and may be a result of relaxed regulatory constraints.

In order to assess the number of unique transcription sites ascribed to unannotated regions, overlapping NSR tag sequences were assembled into contiguous transcription units. Multiple sequencing reads mapping to single genomic sites were collapsed into single transcripts when at least one nucleotide overlapped on either strand. Overall, over 2.5 million transcriptionally active regions were identified that were not covered by current transcript models. Of these, only 21% were supported by sequences in public EST databases (Benson, D. A. et al., Nucleic Acids Res 32:D23-26 (2004)). Unannotated transcription sites averaged 36.9 nucleotides in length and ranged from 32 to 1003 bp, with nearly 5% exceeding 100 bp. Many of the transcriptional elements identified here may represent novel non-coding RNAs. They may also be previously unidentified segments of known genes including alternatively spliced exons and extensions of untranslated regions.

Next, the strand specificity of NSR priming was examined by aligning sequence tags to functional elements of known protein-coding genes. Over 99% of cDNA sequences mapping to protein-coding exons were oriented in the sense orientation, demonstrating the discrimination power of this method for monitoring strand-specific expression. This discrimination power allowed us to determine the orientation of novel transcripts and to assess the prevalence of antisense transcription among the functional elements of known genes. As shown below in TABLE 15, antisense transcription was detected at particularly high levels in 5′UTRs and introns, constituting about 20% of transcription events in those regions.

TABLE 15
The relative frequency ratio of NSR tag sequences oriented in
the sense or antisense direction for sequencing reads obtained
from NSR primed whole brain and UHR libraries

| | Relative frequency | Relative frequency |
|---|---|---|
| Element of | ratio of | ratio of |
| Known genes | Sense Reads | Antisense Reads |
| 5′ UTR | 0.80 | 0.20 |
| coding exon | 0.99 | 0.01 |
| 3′ UTR | 0.95 | 0.05 |
| intron | 0.80 | 0.20 |

The sequencing categories shown above in TABLE 15 were defined by the genomic coordinates of non-coding and coding regions of UCSC known genes.

It is interesting to note that other groups have also documented widespread antisense expression in humans and several model organisms (Katayama S. et al., Science 309:1564-1566 (2005); Ge X. et al., Bioinformatics 22:2475-2479 (2006); Zhang Y. et al., Nucleic Acid Res 34:3465-3475 (2006)). The complex patterns of sense and antisense expression observed in many genes suggest that at least some of the intronic and UTR transcriptional events have functional significance.

### DISCUSSION

As demonstrated in this Example, the application of ultra-high throughput sequencing to NSR-primed cDNA libraries allows for the unbiased interrogation of global transcriptional content that surpasses the scope of information produced by conventional methods. Transcript discovery by sequencing provides information with a level of specificity that cannot be achieved with genomic tiling arrays, which are prone to adverse cross-hybridization effects that necessitate significant data processing and subsequent experimental validation (see.e.g., Royce T. E. et al., Trends Genet. 21:466-475 (2005)). However, the depth of sampling needed to obtain sufficient coverage of rare transcripts in highly complex whole transcriptome libraries limits the capacity of sequencing to rapidly survey large numbers of tissues. In contrast, expression profiling microarrays facilitate the quantitative analysis of transcript levels in many samples, provided there is quality sequence information to direct probe selection.

NSR selective priming provides several advantages over conventional methods. For example, NSR selective priming provides a direct link between informative sequencing and high throughput array experiments. The sequence information obtained using NSR selective primed cDNA libraries allows for the identification of unannotated transcriptional features. The functional characterization of the unannotated transcriptional features identified using the NSR-primed libraries will shed light on a wide range of biological processes and disease states.

The information obtained from high-throughput sequencing may used to inform the design of whole transcriptome arrays for hybridization with NSR-primed cDNA. For example, custom designed whole transcriptome profiling arrays may be used to assess the expression patterns of novel features in relation to one another and in the context of known transcripts. Large scale profiling studies may also be used to implicate individual transcripts in human pathological states and expand the repertoire of biomarkers available for clinical studies (see, e.g., van't Veer, L. J. et al., Nature 415:530-536 (2002)). In addition, the integration of whole transcriptome expression profiling data with genetic linkage analysis may be used to reveal biological activities that are modulated by novel transcriptional elements.

Variations of the tag sequencing method described in this example may be utilized for whole transcriptome analysis in accordance with various embodiments of the invention. In one embodiment, paired-end sequencing is utilized for whole transcriptome analysis. Paired-end sequencing provides a direct physical link between the 5′ and 3′ termini of individual cDNA fragments (Ng P. et al., Nucleic Acids Res 34 e84 (2006); and Campbell, P. J. et al., Nat Genet. 40:722-729 (2008)). Therefore, pair-end sequencing allows spliced exons from distal sites to be unambiguously assigned to a single transcript without any additional information. Once whole transcript structures are defined, large-scale computational analysis can be applied to determine whether these genes represent protein-coding or non-coding RNA entities (Frith M. C. et al., RNA Biol. 3:40-48 (2006)).

As described above, NSR priming is an elementary form of cDNA subtraction with the advantage that it can be simply and reproducibly applied to a wide variety of samples. NSR primer pools may be designed to avoid any population of confounding, hyper-abundant transcripts. For example, an NSR primer pool may be designed to avoid the mRNAs encoding the alpha and beta subunits of globin proteins, which constitute up to 70% of whole blood total RNA mass, and can adversely affect both the sensitivity and accuracy of blood profiling experiments (see Li L. et al., Physiol. Genomics 32:190-197 (2008)). NSR primer pools may also be designed to reduce rRNA content in other organisms, allowing cross-species comparisons of whole transcriptome expression patterns. This approach may be utilized for routine expression profiling experiments in prokaryotic species, where polyA selection of RNA sub-populations is not useful.

In summary, analysis of over 54 million 32-nucleotide tag sequences demonstrated that NSR-priming in the first and second strand cDNA synthesis produces cDNA libraries with broad representation of known poly A+ and poly A− transcripts and dramatically reduced rRNA content when compared to conventional random-priming. The sequencing of NSR-primed libraries provides a global overview of transcription which includes evidence of widespread antisense expression and transcription from previously unannotated genomic sequences. Thus, the simplicity and flexibility of NSR priming technology makes it an ideal companion for ultra-high-throughput sequencing in transcriptome research across a wide range of experimental settings.

While illustrative embodiments have been illustrated and described, it will be appreciated that various changes can be made therein without departing from the spirit and scope of the invention.

1-16. (canceled)

17. A method of transcriptome profiling comprising:
(a) synthesizing a population of single-stranded primer extension products from a target population of nucleic acid molecules within a population of RNA template molecules in a sample isolated from a mammalian subject using reverse transcriptase enzyme and a first population of oligonucleotide primers comprising a hybridizing portion and a first PCR primer binding site located 5′ to the hybridizing portion;
(b) synthesizing double-stranded cDNA from the population of single-stranded primer extension products generated according to step (a) using a DNA polymerase and a second population of oligonucleotide primers comprising a hybridizing portion and a second PCR primer binding site located 5′ to the hybridizing portion, wherein the hybridizing portion is selected from all possible oligonucleotides having a length of 6 nucleotides that hybridize under defined conditions to the target population of nucleic acid molecules and do not hybridize under defined conditions to the non-target population of nucleic acid molecules in the population of single-stranded primer extension products, wherein the non-target population of nucleic acid molecules consists essentially of ribosomal RNA and mitochondrial ribosomal RNA of the same species as the mammalian subject; and
(c) PCR amplifying the double-stranded cDNA synthesized according to step (b) using a first PCR primer that binds to the first PCR primer binding site and a second PCR primer that binds to the second PCR primer binding site.

18. The method of claim 17, further comprising cloning the PCR products into a vector to generate a library representative of the transcriptome of the mammalian subject at the time the sample was isolated.

19. The method of claim 17, further comprising sequencing at least a portion of the PCR products.

20. The method of claim 17, wherein the PCR amplification is carried out using at least 2 cycles of amplification with an annealing temperature between 40 to 50 degrees followed by additional amplification cycles with an annealing temperature of greater than 50 degrees.

21. The method of claim 17, further comprising labeling at least a portion of the amplified PCR products.

22. The method of claim 17, wherein the first PCR primer binding site of each oligonucleotide in the first population comprises a region of at least 8 consecutive nucleotides that are identical to a region of at least 8 consecutive nucleotides in the second PCR primer binding site of each oligonucleotide in the second population of oligonucleotides.

23. The method of claim 17, wherein the PCR primer binding site of at least one of the first or second population of oligonucleotides comprises an RNA portion and a DNA portion, wherein the RNA portion is 5′ with respect to the DNA portion.

24. A population of amplified nucleic acid molecules generated using the method of claim 17.

25. A method of selectively amplifying a target population of nucleic acid molecules within a larger non-target population of nucleic acid molecules, the method comprising the steps of:
(a) synthesizing single-stranded cDNA from a sample comprising total RNA isolated from a mammalian subject using reverse transcriptase enzyme and a first population of oligonucleotide primers, and wherein each oligonucleotide within the first population of oligonucleotide primers comprises a hybridizing portion and a defined sequence portion located 5′ to the hybridizing portion, wherein the hybridizing portion is a member of the population of oligonucleotides comprising SEQ ID NOS:1-749; and
(b) synthesizing double-stranded cDNA from the single-stranded cDNA synthesized according to step (a) using a DNA polymerase and a second population of oligonucleotide primers, wherein each oligonucleotide within the second population of oligonucleotide primers comprises a hybridizing portion and a defined sequence portion located 5′ to the hybridizing portion, and wherein the hybridizing portion is a member of the population of oligonucleotides comprising SEQ ID NOS:750-1498.

26. The method of claim 25, wherein the population of hybridizing portions of the first population of oligonucleotide primers comprises at least 10% of the oligonucleotides comprising SEQ ID NOS:1-749.

27. The method of claim 25, wherein the population of hybridizing portions of the second population of oligonucleotide primers comprises at least 10% of the oligonucleotides comprising SEQ ID NOS:750-1498.

28. The method of claim 25, further comprising sequencing at least a portion of the PCR products.

29. The method of claim 25, further comprising labeling at least a portion of the PCR products.

30. A population of oligonucleotides comprising SEQ ID NOS:1-749 for use in first strand cDNA synthesis.

31. A population of oligonucleotides comprising SEQ ID NOS:750-1498 for use in second strand cDNA synthesis.

32. A reagent for selectively amplifying a target population of nucleic acid molecules, the reagent comprising at least 10% of the oligonucleotides comprising SEQ ID NOS:1-749.

33. A reagent for selectively amplifying a target population of nucleic acid molecules, the reagent comprising at least 10% of the oligonucleotides comprising SEQ ID NOS:750-1498.

34. A reagent for selectively amplifying a target population of nucleic acid molecules, the reagent comprising a population of oligonucleotides to prime the amplification of a target population of nucleic acid molecules wherein each oligonucleotide comprises a hybridizing portion and a defined sequence portion located 5′ to the hybridizing portion, wherein the hybridizing portion is a member of the population of oligonucleotides comprising SEQ ID NOS:1-749.

35. A reagent for selectively amplifying a target population of nucleic acid molecules, the reagent comprising a population of oligonucleotides to prime the amplification of a target population of nucleic acid molecules wherein each oligonucleotide comprises a hybridizing portion and a defined sequence portion located 5′ to the hybridizing portion, wherein the hybridizing portion is a member of the population of oligonucleotides comprising SEQ ID NOS:750-1498.

36. A kit for selectively amplifying a target population of nucleic acid molecules, the kit comprising a reagent comprising a first population of oligonucleotides for first strand cDNA synthesis wherein each oligonucleotide in the first population of oligonucleotides comprises a hybridizing portion and a defined sequence portion located 5′ to the hybridizing portion, wherein the hybridizing portion is a member of the population of oligonucleotides comprising SEQ ID NOS:1-749.

37. The kit of claim 36, wherein the population of hybridizing portions in the first population of oligonucleotides comprises at least 10% of the oligonucleotides comprising SEQ ID NOS:1-749.

38. The kit of claim 36, further comprising a second population of oligonucleotides for second strand cDNA synthesis, wherein each oligonucleotide in the second population of oligonucleotides comprises a hybridizing portion and a defined sequence portion located 5′ to the hybridizing portion, wherein the hybridizing portion is a member of the population of oligonucleotides comprising SEQ ID NOS:750-1498.

39. The kit of claim 38, wherein the population of hybridizing portions in the second population of oligonucleotides comprises at least 10% of the oligonucleotides comprising SEQ ID NOS:750-1498.

40. The kit of claim 38, wherein the population of hybridizing portions in the first population of oligonucleotides comprises the oligonucleotides consisting of SEQ ID NOS:1-749 and wherein the population of hybridizing portions in the second population of oligonucleotides comprises the oligonucleotides consisting of SEQ ID NOS:750-1498.

41. The kit of claim 38, further comprising at least one of the following components: a reverse transcriptase, a DNA polymerase, a DNA ligase, a RNase H enzyme, a Tris buffer, a potassium salt, a magnesium salt, an ammonium salt, a reducing agent, deoxynucleoside triphosphates, or a ribonuclease inhibitor.

42. A kit for selectively amplifying a target population of nucleic acid molecules within a population of RNA template molecules in a sample obtained from a mammalian subject, the kit comprising:
(a) a first population of oligonucleotide primers comprising a hybridizing portion consisting of 6 nucleotides selected from all possible oligonucleotides having a length of 6 nucleotides that do not hybridize under defined conditions to the non-target population of nucleic acid molecules in the population of RNA templatemolecules, and a defined sequence portion located 5′ to the hybridizing portion, wherein the non-target population of nucleic acid molecules consists essentially of the most abundant nucleic acid molecules in the population of RNA template molecules;
(b) a second population of oligonucleotide primers comprising a hybridizing portion consisting of 6 nucleotides selected from the reverse complement of the nucleotide sequence of the hybridizing portion of the first population of oligonucleotide primers, and a defined sequence portion located 5′ to the hybridizing portion;
(c) a first PCR primer that binds to the first defined sequence portion of the first population of oligonucleotides and a second PCR primer that binds to the second defined sequence portion of the second population of oligonucleotides.

43. The kit of claim 42, wherein the non-target population of nucleic acid molecules consists essentially of ribosomal RNA and mitochondrial ribosomal RNA of the same species as the mammalian subject.

44. The kit of claim 42, wherein the defined sequence portion of each oligonucleotide in the first and second population of oligonucleotides consists of a primer binding site for PCR amplification ranging in length from 10 nucleotides to 20 nucleotides.

45. The kit of claim 42, wherein the defined sequence portion of each oligonucleotide in the first population comprises a region of at least 8 consecutive nucleotides that are identical to a region of at least 8 consecutive nucleotides in the defined sequence portion of each oligonucleotide in the second population.

46. The kit of claim 42, wherein the defined sequence portion of at least one of the first or second population of oligonucleotides comprises an RNA portion and a DNA portion, wherein the RNA portion is 5′ with respect to the DNA portion.

47. A method of selectively amplifying a target population of nucleic acid molecules to generate amplified DNA molecules, the method comprising the steps of:
(a) providing a first population of oligonucleotides wherein each oligonucleotide comprises a hybridizing portion and a first PCR primer binding sitelocated 5′ to the hybridizing portion, wherein the hybridizing portion is a member of the population of oligonucleotides comprising SEQ ID NOS:1-749;
(b) annealing the first population of oligonucleotides to a sample comprising RNA isolated from a mammalian subject;
(c) synthesizing cDNA from the RNA using a reverse transcriptase enzyme;
(d) synthesizing double-stranded cDNA using a DNA polymerase and a second population of oligonucleotides, wherein each oligonucleotide comprises a hybridizing portion and a second PCR binding site located 5′ to the hybridizing portion, wherein the hybridizing portion is a member of the population of oligonucleotides comprising SEQ ID NOS:750-1498;
(e) PCR amplifying the double stranded cDNA using thermostable DNA polymerase, a first PCR primer that binds to the first PCR primer binding site and a second PCR primer that binds to the second PCR primer binding site to generate amplified double stranded DNA; and
(f) sequencing the amplified double-stranded PCR products.

48. A population of selectively amplified nucleic acid molecules consisting of a representation of a target population of nucleic acid molecules within a population of RNA template molecules in a cell sample isolated from a mammalian subject, each amplified nucleic acid molecule comprising:
a 5′ defined sequence portion flanking a member of the population of amplified nucleic acid sequences, and a 3′ defined sequence, wherein the population of selectively amplified sequences includes an amplified nucleic acid sequence corresponding to a target RNA molecule expressed in the mammalian cell, and is characterized by having the following properties with reference to the particular mammalian species:
(a) having greater than 75% polyadenylated and non-polyadenylated transcripts; and having less than 10% ribosomal RNA.

49. The population of claim 48 inserted into a cloning vector.

50. The population of claim 48, wherein each nucleic acid molecule in the population is labeled.

51. The population of claim 48 attached to a substrate.

52. The population of claim 48, wherein the defined sequence portion of at least one of the first or second population of oligonucleotides comprises an RNA portion and a DNA portion, wherein the RNA portion is 5′ with respect to the DNA portion.


SEQUENCE LISTING (1560 sequences)

SEQ ID NO: 1 - DNA, 6 bp, Artificial Sequence
  synthetic
acgttt

SEQ ID NO: 2 - DNA, 6 bp, Artificial Sequence
  synthetic
ccggtt

SEQ ID NO: 3 - DNA, 6 bp, Artificial Sequence
  synthetic
gtcgtt

SEQ ID NO: 4 - DNA, 6 bp, Artificial Sequence
  synthetic
tacgtt

SEQ ID NO: 5 - DNA, 6 bp, Artificial Sequence
  synthetic
aacgtt

SEQ ID NO: 6 - DNA, 6 bp, Artificial Sequence
  synthetic
tgtctt

SEQ ID NO: 7 - DNA, 6 bp, Artificial Sequence
  synthetic
gatctt

SEQ ID NO: 8 - DNA, 6 bp, Artificial Sequence
  synthetic
cccctt

SEQ ID NO: 9 - DNA, 6 bp, Artificial Sequence
  synthetic
ctactt

SEQ ID NO: 10 - DNA, 6 bp, Artificial Sequence
  synthetic
cgtatt

SEQ ID NO: 11 - DNA, 6 bp, Artificial Sequence
  synthetic
agtatt

SEQ ID NO: 12 - DNA, 6 bp, Artificial Sequence
  synthetic
catatt

SEQ ID NO: 13 - DNA, 6 bp, Artificial Sequence
  synthetic
atgatt

SEQ ID NO: 14 - DNA, 6 bp, Artificial Sequence
  synthetic
gcgatt

SEQ ID NO: 15 - DNA, 6 bp, Artificial Sequence
  synthetic
acgatt

SEQ ID NO: 16 - DNA, 6 bp, Artificial Sequence
  synthetic
cagatt

SEQ ID NO: 17 - DNA, 6 bp, Artificial Sequence
  synthetic
gtcatt

SEQ ID NO: 18 - DNA, 6 bp, Artificial Sequence
  synthetic
agcatt

SEQ ID NO: 19 - DNA, 6 bp, Artificial Sequence
  synthetic
accatt

SEQ ID NO: 20 - DNA, 6 bp, Artificial Sequence
  synthetic
gcaatt

SEQ ID NO: 21 - DNA, 6 bp, Artificial Sequence
  synthetic
acaatt

SEQ ID NO: 22 - DNA, 6 bp, Artificial Sequence
  synthetic
aaaatt

SEQ ID NO: 23 - DNA, 6 bp, Artificial Sequence
  synthetic
tgttgt

SEQ ID NO: 24 - DNA, 6 bp, Artificial Sequence
  synthetic
cgttgt

SEQ ID NO: 25 - DNA, 6 bp, Artificial Sequence
  synthetic
gcgtgt

SEQ ID NO: 26 - DNA, 6 bp, Artificial Sequence
  synthetic
gactgt

SEQ ID NO: 27 - DNA, 6 bp, Artificial Sequence
  synthetic
gtatgt

SEQ ID NO: 28 - DNA, 6 bp, Artificial Sequence
  synthetic
ctatgt

SEQ ID NO: 29 - DNA, 6 bp, Artificial Sequence
  synthetic
cgatgt

SEQ ID NO: 30 - DNA, 6 bp, Artificial Sequence
  synthetic
agatgt

SEQ ID NO: 31 - DNA, 6 bp, Artificial Sequence
  synthetic
taatgt

SEQ ID NO: 32 - DNA, 6 bp, Artificial Sequence
  synthetic
caatgt

SEQ ID NO: 33 - DNA, 6 bp, Artificial Sequence
  synthetic
gctggt

SEQ ID NO: 34 - DNA, 6 bp, Artificial Sequence
  synthetic
gtcggt

SEQ ID NO: 35 - DNA, 6 bp, Artificial Sequence
  synthetic
accggt

SEQ ID NO: 36 - DNA, 6 bp, Artificial Sequence
  synthetic
cttcgt

SEQ ID NO: 37 - DNA, 6 bp, Artificial Sequence
  synthetic
cgtcgt

SEQ ID NO: 38 - DNA, 6 bp, Artificial Sequence
  synthetic
agtcgt

SEQ ID NO: 39 - DNA, 6 bp, Artificial Sequence
  synthetic
ttgcgt

SEQ ID NO: 40 - DNA, 6 bp, Artificial Sequence
  synthetic
ccgcgt

SEQ ID NO: 41 - DNA, 6 bp, Artificial Sequence
  synthetic
acgcgt

SEQ ID NO: 42 - DNA, 6 bp, Artificial Sequence
  synthetic
ctccgt

SEQ ID NO: 43 - DNA, 6 bp, Artificial Sequence
  synthetic
atccgt

SEQ ID NO: 44 - DNA, 6 bp, Artificial Sequence
  synthetic
aaccgt

SEQ ID NO: 45 - DNA, 6 bp, Artificial Sequence
  synthetic
gtacgt

SEQ ID NO: 46 - DNA, 6 bp, Artificial Sequence
  synthetic
atacgt

SEQ ID NO: 47 - DNA, 6 bp, Artificial Sequence
  synthetic
ggacgt

SEQ ID NO: 48 - DNA, 6 bp, Artificial Sequence
  synthetic
caacgt

SEQ ID NO: 49 - DNA, 6 bp, Artificial Sequence
  synthetic
aaacgt

SEQ ID NO: 50 - DNA, 6 bp, Artificial Sequence
  synthetic
tgtagt

SEQ ID NO: 51 - DNA, 6 bp, Artificial Sequence
  synthetic
aatagt

SEQ ID NO: 52 - DNA, 6 bp, Artificial Sequence
  synthetic
atgagt

SEQ ID NO: 53 - DNA, 6 bp, Artificial Sequence
  synthetic
cggagt

SEQ ID NO: 54 - DNA, 6 bp, Artificial Sequence
  synthetic
tcgagt

SEQ ID NO: 55 - DNA, 6 bp, Artificial Sequence
  synthetic
acgagt

SEQ ID NO: 56 - DNA, 6 bp, Artificial Sequence
  synthetic
tacagt

SEQ ID NO: 57 - DNA, 6 bp, Artificial Sequence
  synthetic
gtaagt

SEQ ID NO: 58 - DNA, 6 bp, Artificial Sequence
  synthetic
ataagt

SEQ ID NO: 59 - DNA, 6 bp, Artificial Sequence
  synthetic
gcaagt

SEQ ID NO: 60 - DNA, 6 bp, Artificial Sequence
  synthetic
cgatct

SEQ ID NO: 61 - DNA, 6 bp, Artificial Sequence
  synthetic
catgct

SEQ ID NO: 62 - DNA, 6 bp, Artificial Sequence
  synthetic
ttgcct

SEQ ID NO: 63 - DNA, 6 bp, Artificial Sequence
  synthetic
acccct

SEQ ID NO: 64 - DNA, 6 bp, Artificial Sequence
  synthetic
gtacct

SEQ ID NO: 65 - DNA, 6 bp, Artificial Sequence
  synthetic
atacct

SEQ ID NO: 66 - DNA, 6 bp, Artificial Sequence
  synthetic
caacct

SEQ ID NO: 67 - DNA, 6 bp, Artificial Sequence
  synthetic
agtact

SEQ ID NO: 68 - DNA, 6 bp, Artificial Sequence
  synthetic
actact

SEQ ID NO: 69 - DNA, 6 bp, Artificial Sequence
  synthetic
gtgact

SEQ ID NO: 70 - DNA, 6 bp, Artificial Sequence
  synthetic
gagact

SEQ ID NO: 71 - DNA, 6 bp, Artificial Sequence
  synthetic
ctaact

SEQ ID NO: 72 - DNA, 6 bp, Artificial Sequence
  synthetic
cgaact

SEQ ID NO: 73 - DNA, 6 bp, Artificial Sequence
  synthetic
aattat

SEQ ID NO: 74 - DNA, 6 bp, Artificial Sequence
  synthetic
gtgtat

SEQ ID NO: 75 - DNA, 6 bp, Artificial Sequence
  synthetic
tcgtat

SEQ ID NO: 76 - DNA, 6 bp, Artificial Sequence
  synthetic
gcgtat

SEQ ID NO: 77 - DNA, 6 bp, Artificial Sequence
  synthetic
acgtat

SEQ ID NO: 78 - DNA, 6 bp, Artificial Sequence
  synthetic
cagtat

SEQ ID NO: 79 - DNA, 6 bp, Artificial Sequence
  synthetic
aactat

SEQ ID NO: 80 - DNA, 6 bp, Artificial Sequence
  synthetic
atatat

SEQ ID NO: 81 - DNA, 6 bp, Artificial Sequence
  synthetic
cgatat

SEQ ID NO: 82 - DNA, 6 bp, Artificial Sequence
  synthetic
tcatat

SEQ ID NO: 83 - DNA, 6 bp, Artificial Sequence
  synthetic
ccatat

SEQ ID NO: 84 - DNA, 6 bp, Artificial Sequence
  synthetic
acatat

SEQ ID NO: 85 - DNA, 6 bp, Artificial Sequence
  synthetic
caatat

SEQ ID NO: 86 - DNA, 6 bp, Artificial Sequence
  synthetic
aaatat

SEQ ID NO: 87 - DNA, 6 bp, Artificial Sequence
  synthetic
cgtgat

SEQ ID NO: 88 - DNA, 6 bp, Artificial Sequence
  synthetic
tatgat

SEQ ID NO: 89 - DNA, 6 bp, Artificial Sequence
  synthetic
gtggat

SEQ ID NO: 90 - DNA, 6 bp, Artificial Sequence
  synthetic
gaggat

SEQ ID NO: 91 - DNA, 6 bp, Artificial Sequence
  synthetic
gtcgat

SEQ ID NO: 92 - DNA, 6 bp, Artificial Sequence
  synthetic
agcgat

SEQ ID NO: 93 - DNA, 6 bp, Artificial Sequence
  synthetic
tccgat

SEQ ID NO: 94 - DNA, 6 bp, Artificial Sequence
  synthetic
tacgat

SEQ ID NO: 95 - DNA, 6 bp, Artificial Sequence
  synthetic
gacgat

SEQ ID NO: 96 - DNA, 6 bp, Artificial Sequence
  synthetic
cacgat

SEQ ID NO: 97 - DNA, 6 bp, Artificial Sequence
  synthetic
ggagat

SEQ ID NO: 98 - DNA, 6 bp, Artificial Sequence
  synthetic
agagat

SEQ ID NO: 99 - DNA, 6 bp, Artificial Sequence
  synthetic
gcagat

SEQ ID NO: 100 - DNA, 6 bp, Artificial Sequence
  synthetic
tgtcat

SEQ ID NO: 101 - DNA, 6 bp, Artificial Sequence
  synthetic
cgtcat

SEQ ID NO: 102 - DNA, 6 bp, Artificial Sequence
  synthetic
gctcat

SEQ ID NO: 103 - DNA, 6 bp, Artificial Sequence
  synthetic
gtgcat

SEQ ID NO: 104 - DNA, 6 bp, Artificial Sequence
  synthetic
gcgcat

SEQ ID NO: 105 - DNA, 6 bp, Artificial Sequence
  synthetic
tcccat

SEQ ID NO: 106 - DNA, 6 bp, Artificial Sequence
  synthetic
gaccat

SEQ ID NO: 107 - DNA, 6 bp, Artificial Sequence
  synthetic
ttacat

SEQ ID NO: 108 - DNA, 6 bp, Artificial Sequence
  synthetic
gtacat

SEQ ID NO: 109 - DNA, 6 bp, Artificial Sequence
  synthetic
atacat

SEQ ID NO: 110 - DNA, 6 bp, Artificial Sequence
  synthetic
cgtaat

SEQ ID NO: 111 - DNA, 6 bp, Artificial Sequence
  synthetic
cctaat

SEQ ID NO: 112 - DNA, 6 bp, Artificial Sequence
  synthetic
gataat

SEQ ID NO: 113 - DNA, 6 bp, Artificial Sequence
  synthetic
atgaat

SEQ ID NO: 114 - DNA, 6 bp, Artificial Sequence
  synthetic
ccgaat

SEQ ID NO: 115 - DNA, 6 bp, Artificial Sequence
  synthetic
ggcaat

SEQ ID NO: 116 - DNA, 6 bp, Artificial Sequence
  synthetic
agcaat

SEQ ID NO: 117 - DNA, 6 bp, Artificial Sequence
  synthetic
cccaat

SEQ ID NO: 118 - DNA, 6 bp, Artificial Sequence
  synthetic
accaat

SEQ ID NO: 119 - DNA, 6 bp, Artificial Sequence
  synthetic
tacaat

SEQ ID NO: 120 - DNA, 6 bp, Artificial Sequence
  synthetic
gacaat

SEQ ID NO: 121 - DNA, 6 bp, Artificial Sequence
  synthetic
taaaat

SEQ ID NO: 122 - DNA, 6 bp, Artificial Sequence
  synthetic
aaaaat

SEQ ID NO: 123 - DNA, 6 bp, Artificial Sequence
  synthetic
atgttg

SEQ ID NO: 124 - DNA, 6 bp, Artificial Sequence
  synthetic
cggttg

SEQ ID NO: 125 - DNA, 6 bp, Artificial Sequence
  synthetic
cgattg

SEQ ID NO: 126 - DNA, 6 bp, Artificial Sequence
  synthetic
gaattg

SEQ ID NO: 127 - DNA, 6 bp, Artificial Sequence
  synthetic
actgtg

SEQ ID NO: 128 - DNA, 6 bp, Artificial Sequence
  synthetic
tatgtg

SEQ ID NO: 129 - DNA, 6 bp, Artificial Sequence
  synthetic
aatgtg

SEQ ID NO: 130 - DNA, 6 bp, Artificial Sequence
  synthetic
tcggtg

SEQ ID NO: 131 - DNA, 6 bp, Artificial Sequence
  synthetic
taggtg

SEQ ID NO: 132 - DNA, 6 bp, Artificial Sequence
  synthetic
gtcgtg

SEQ ID NO: 133 - DNA, 6 bp, Artificial Sequence
  synthetic
tgagtg

SEQ ID NO: 134 - DNA, 6 bp, Artificial Sequence
  synthetic
agagtg

SEQ ID NO: 135 - DNA, 6 bp, Artificial Sequence
  synthetic
ccagtg

SEQ ID NO: 136 - DNA, 6 bp, Artificial Sequence
  synthetic
taagtg

SEQ ID NO: 137 - DNA, 6 bp, Artificial Sequence
  synthetic
actctg

SEQ ID NO: 138 - DNA, 6 bp, Artificial Sequence
  synthetic
catctg

SEQ ID NO: 139 - DNA, 6 bp, Artificial Sequence
  synthetic
atgctg

SEQ ID NO: 140 - DNA, 6 bp, Artificial Sequence
  synthetic
ttcctg

SEQ ID NO: 141 - DNA, 6 bp, Artificial Sequence
  synthetic
tacctg

SEQ ID NO: 142 - DNA, 6 bp, Artificial Sequence
  synthetic
agactg

SEQ ID NO: 143 - DNA, 6 bp, Artificial Sequence
  synthetic
gaactg

SEQ ID NO: 144 - DNA, 6 bp, Artificial Sequence
  synthetic
tgtatg

SEQ ID NO: 145 - DNA, 6 bp, Artificial Sequence
  synthetic
cgtatg

SEQ ID NO: 146 - DNA, 6 bp, Artificial Sequence
  synthetic
agtatg

SEQ ID NO: 147 - DNA, 6 bp, Artificial Sequence
  synthetic
tctatg

SEQ ID NO: 148 - DNA, 6 bp, Artificial Sequence
  synthetic
cctatg

SEQ ID NO: 149 - DNA, 6 bp, Artificial Sequence
  synthetic
cggatg

SEQ ID NO: 150 - DNA, 6 bp, Artificial Sequence
  synthetic
aggatg

SEQ ID NO: 151 - DNA, 6 bp, Artificial Sequence
  synthetic
tcgatg

SEQ ID NO: 152 - DNA, 6 bp, Artificial Sequence
  synthetic
ccgatg

SEQ ID NO: 153 - DNA, 6 bp, Artificial Sequence
  synthetic
acgatg

SEQ ID NO: 154 - DNA, 6 bp, Artificial Sequence
  synthetic
cgcatg

SEQ ID NO: 155 - DNA, 6 bp, Artificial Sequence
  synthetic
tacatg

SEQ ID NO: 156 - DNA, 6 bp, Artificial Sequence
  synthetic
gtaatg

SEQ ID NO: 157 - DNA, 6 bp, Artificial Sequence
  synthetic
ctaatg

SEQ ID NO: 158 - DNA, 6 bp, Artificial Sequence
  synthetic
tgaatg

SEQ ID NO: 159 - DNA, 6 bp, Artificial Sequence
  synthetic
gcaatg

SEQ ID NO: 160 - DNA, 6 bp, Artificial Sequence
  synthetic
ggatgg

SEQ ID NO: 161 - DNA, 6 bp, Artificial Sequence
  synthetic
cgatgg

SEQ ID NO: 162 - DNA, 6 bp, Artificial Sequence
  synthetic
taatgg

SEQ ID NO: 163 - DNA, 6 bp, Artificial Sequence
  synthetic
aagcgg

SEQ ID NO: 164 - DNA, 6 bp, Artificial Sequence
  synthetic
aaccgg

SEQ ID NO: 165 - DNA, 6 bp, Artificial Sequence
  synthetic
atacgg

SEQ ID NO: 166 - DNA, 6 bp, Artificial Sequence
  synthetic
tgtagg

SEQ ID NO: 167 - DNA, 6 bp, Artificial Sequence
  synthetic
tgaagg

SEQ ID NO: 168 - DNA, 6 bp, Artificial Sequence
  synthetic
atttcg

SEQ ID NO: 169 - DNA, 6 bp, Artificial Sequence
  synthetic
tgttcg

SEQ ID NO: 170 - DNA, 6 bp, Artificial Sequence
  synthetic
aattcg

SEQ ID NO: 171 - DNA, 6 bp, Artificial Sequence
  synthetic
ctgtcg

SEQ ID NO: 172 - DNA, 6 bp, Artificial Sequence
  synthetic
tagtcg

SEQ ID NO: 173 - DNA, 6 bp, Artificial Sequence
  synthetic
gagtcg

SEQ ID NO: 174 - DNA, 6 bp, Artificial Sequence
  synthetic
atatcg

SEQ ID NO: 175 - DNA, 6 bp, Artificial Sequence
  synthetic
tcatcg

SEQ ID NO: 176 - DNA, 6 bp, Artificial Sequence
  synthetic
gatgcg

SEQ ID NO: 177 - DNA, 6 bp, Artificial Sequence
  synthetic
aacgcg

SEQ ID NO: 178 - DNA, 6 bp, Artificial Sequence
  synthetic
catccg

SEQ ID NO: 179 - DNA, 6 bp, Artificial Sequence
  synthetic
aatccg

SEQ ID NO: 180 - DNA, 6 bp, Artificial Sequence
  synthetic
atgccg

SEQ ID NO: 181 - DNA, 6 bp, Artificial Sequence
  synthetic
aggccg

SEQ ID NO: 182 - DNA, 6 bp, Artificial Sequence
  synthetic
ataccg

SEQ ID NO: 183 - DNA, 6 bp, Artificial Sequence
  synthetic
agaccg

SEQ ID NO: 184 - DNA, 6 bp, Artificial Sequence
  synthetic
taaccg

SEQ ID NO: 185 - DNA, 6 bp, Artificial Sequence
  synthetic
attacg

SEQ ID NO: 186 - DNA, 6 bp, Artificial Sequence
  synthetic
agtacg

SEQ ID NO: 187 - DNA, 6 bp, Artificial Sequence
  synthetic
gatacg

SEQ ID NO: 188 - DNA, 6 bp, Artificial Sequence
  synthetic
catacg

SEQ ID NO: 189 - DNA, 6 bp, Artificial Sequence
  synthetic
tcgacg

SEQ ID NO: 190 - DNA, 6 bp, Artificial Sequence
  synthetic
gtcacg

SEQ ID NO: 191 - DNA, 6 bp, Artificial Sequence
  synthetic
tacacg

SEQ ID NO: 192 - DNA, 6 bp, Artificial Sequence
  synthetic
acaacg

SEQ ID NO: 193 - DNA, 6 bp, Artificial Sequence
  synthetic
gaaacg

SEQ ID NO: 194 - DNA, 6 bp, Artificial Sequence
  synthetic
ctttag

SEQ ID NO: 195 - DNA, 6 bp, Artificial Sequence
  synthetic
cgttag

SEQ ID NO: 196 - DNA, 6 bp, Artificial Sequence
  synthetic
ctgtag

SEQ ID NO: 197 - DNA, 6 bp, Artificial Sequence
  synthetic
ccgtag

SEQ ID NO: 198 - DNA, 6 bp, Artificial Sequence
  synthetic
gtctag

SEQ ID NO: 199 - DNA, 6 bp, Artificial Sequence
  synthetic
cgctag

SEQ ID NO: 200 - DNA, 6 bp, Artificial Sequence
  synthetic
agctag

SEQ ID NO: 201 - DNA, 6 bp, Artificial Sequence
  synthetic
gcctag

SEQ ID NO: 202 - DNA, 6 bp, Artificial Sequence
  synthetic
ttatag

SEQ ID NO: 203 - DNA, 6 bp, Artificial Sequence
  synthetic
cgatag

SEQ ID NO: 204 - DNA, 6 bp, Artificial Sequence
  synthetic
ttcgag

SEQ ID NO: 205 - DNA, 6 bp, Artificial Sequence
  synthetic
ctcgag

SEQ ID NO: 206 - DNA, 6 bp, Artificial Sequence
  synthetic
aacgag

SEQ ID NO: 207 - DNA, 6 bp, Artificial Sequence
  synthetic
gtagag

SEQ ID NO: 208 - DNA, 6 bp, Artificial Sequence
  synthetic
atagag

SEQ ID NO: 209 - DNA, 6 bp, Artificial Sequence
  synthetic
tgagag

SEQ ID NO: 210 - DNA, 6 bp, Artificial Sequence
  synthetic
acagag

SEQ ID NO: 211 - DNA, 6 bp, Artificial Sequence
  synthetic
aatcag

SEQ ID NO: 212 - DNA, 6 bp, Artificial Sequence
  synthetic
gcgcag

SEQ ID NO: 213 - DNA, 6 bp, Artificial Sequence
  synthetic
taccag

SEQ ID NO: 214 - DNA, 6 bp, Artificial Sequence
  synthetic
ctacag

SEQ ID NO: 215 - DNA, 6 bp, Artificial Sequence
  synthetic
cgacag

SEQ ID NO: 216 - DNA, 6 bp, Artificial Sequence
  synthetic
gcacag

SEQ ID NO: 217 - DNA, 6 bp, Artificial Sequence
  synthetic
gttaag

SEQ ID NO: 218 - DNA, 6 bp, Artificial Sequence
  synthetic
tgtaag

SEQ ID NO: 219 - DNA, 6 bp, Artificial Sequence
  synthetic
cgtaag

SEQ ID NO: 220 - DNA, 6 bp, Artificial Sequence
  synthetic
cctaag

SEQ ID NO: 221 - DNA, 6 bp, Artificial Sequence
  synthetic
tataag

SEQ ID NO: 222 - DNA, 6 bp, Artificial Sequence
  synthetic
gataag

SEQ ID NO: 223 - DNA, 6 bp, Artificial Sequence
  synthetic
aataag

SEQ ID NO: 224 - DNA, 6 bp, Artificial Sequence
  synthetic
tggaag

SEQ ID NO: 225 - DNA, 6 bp, Artificial Sequence
  synthetic
tagaag

SEQ ID NO: 226 - DNA, 6 bp, Artificial Sequence
  synthetic
gagaag

SEQ ID NO: 227 - DNA, 6 bp, Artificial Sequence
  synthetic
gtaaag

SEQ ID NO: 228 - DNA, 6 bp, Artificial Sequence
  synthetic
gatttc

SEQ ID NO: 229 - DNA, 6 bp, Artificial Sequence
  synthetic
atattc

SEQ ID NO: 230 - DNA, 6 bp, Artificial Sequence
  synthetic
cgattc

SEQ ID NO: 231 - DNA, 6 bp, Artificial Sequence
  synthetic
tacgtc

SEQ ID NO: 232 - DNA, 6 bp, Artificial Sequence
  synthetic
ctagtc

SEQ ID NO: 233 - DNA, 6 bp, Artificial Sequence
  synthetic
cgagtc

SEQ ID NO: 234 - DNA, 6 bp, Artificial Sequence
  synthetic
caagtc

SEQ ID NO: 235 - DNA, 6 bp, Artificial Sequence
  synthetic
aaagtc

SEQ ID NO: 236 - DNA, 6 bp, Artificial Sequence
  synthetic
attctc

SEQ ID NO: 237 - DNA, 6 bp, Artificial Sequence
  synthetic
cgtctc

SEQ ID NO: 238 - DNA, 6 bp, Artificial Sequence
  synthetic
tatctc

SEQ ID NO: 239 - DNA, 6 bp, Artificial Sequence
  synthetic
agcctc

SEQ ID NO: 240 - DNA, 6 bp, Artificial Sequence
  synthetic
gtactc

SEQ ID NO: 241 - DNA, 6 bp, Artificial Sequence
  synthetic
tgactc

SEQ ID NO: 242 - DNA, 6 bp, Artificial Sequence
  synthetic
taactc

SEQ ID NO: 243 - DNA, 6 bp, Artificial Sequence
  synthetic
attatc

SEQ ID NO: 244 - DNA, 6 bp, Artificial Sequence
  synthetic
tgtatc

SEQ ID NO: 245 - DNA, 6 bp, Artificial Sequence
  synthetic
agtatc

SEQ ID NO: 246 - DNA, 6 bp, Artificial Sequence
  synthetic
catatc

SEQ ID NO: 247 - DNA, 6 bp, Artificial Sequence
  synthetic
gtcatc

SEQ ID NO: 248 - DNA, 6 bp, Artificial Sequence
  synthetic
ctcatc

SEQ ID NO: 249 - DNA, 6 bp, Artificial Sequence
  synthetic
tccatc

SEQ ID NO: 250 - DNA, 6 bp, Artificial Sequence
  synthetic
tacatc

SEQ ID NO: 251 - DNA, 6 bp, Artificial Sequence
  synthetic
cgaatc

SEQ ID NO: 252 - DNA, 6 bp, Artificial Sequence
  synthetic
tgttgc

SEQ ID NO: 253 - DNA, 6 bp, Artificial Sequence
  synthetic
ctgtgc

SEQ ID NO: 254 - DNA, 6 bp, Artificial Sequence
  synthetic
tagtgc

SEQ ID NO: 255 - DNA, 6 bp, Artificial Sequence
  synthetic
gtatgc

SEQ ID NO: 256 - DNA, 6 bp, Artificial Sequence
  synthetic
ctatgc

SEQ ID NO: 257 - DNA, 6 bp, Artificial Sequence
  synthetic
caatgc

SEQ ID NO: 258 - DNA, 6 bp, Artificial Sequence
  synthetic
gttggc

SEQ ID NO: 259 - DNA, 6 bp, Artificial Sequence
  synthetic
aatggc

SEQ ID NO: 260 - DNA, 6 bp, Artificial Sequence
  synthetic
taaggc

SEQ ID NO: 261 - DNA, 6 bp, Artificial Sequence
  synthetic
agtcgc

SEQ ID NO: 262 - DNA, 6 bp, Artificial Sequence
  synthetic
aagcgc

SEQ ID NO: 263 - DNA, 6 bp, Artificial Sequence
  synthetic
ctacgc

SEQ ID NO: 264 - DNA, 6 bp, Artificial Sequence
  synthetic
gctagc

SEQ ID NO: 265 - DNA, 6 bp, Artificial Sequence
  synthetic
actagc

SEQ ID NO: 266 - DNA, 6 bp, Artificial Sequence
  synthetic
gatagc

SEQ ID NO: 267 - DNA, 6 bp, Artificial Sequence
  synthetic
catagc

SEQ ID NO: 268 - DNA, 6 bp, Artificial Sequence
  synthetic
tcgagc

SEQ ID NO: 269 - DNA, 6 bp, Artificial Sequence
  synthetic
atcagc

SEQ ID NO: 270 - DNA, 6 bp, Artificial Sequence
  synthetic
tacagc

SEQ ID NO: 271 - DNA, 6 bp, Artificial Sequence
  synthetic
cacagc

SEQ ID NO: 272 - DNA, 6 bp, Artificial Sequence
  synthetic
gtaagc

SEQ ID NO: 273 - DNA, 6 bp, Artificial Sequence
  synthetic
ataagc

SEQ ID NO: 274 - DNA, 6 bp, Artificial Sequence
  synthetic
gcttcc

SEQ ID NO: 275 - DNA, 6 bp, Artificial Sequence
  synthetic
acgtcc

SEQ ID NO: 276 - DNA, 6 bp, Artificial Sequence
  synthetic
aagtcc

SEQ ID NO: 277 - DNA, 6 bp, Artificial Sequence
  synthetic
gatgcc

SEQ ID NO: 278 - DNA, 6 bp, Artificial Sequence
  synthetic
ctagcc

SEQ ID NO: 279 - DNA, 6 bp, Artificial Sequence
  synthetic
atagcc

SEQ ID NO: 280 - DNA, 6 bp, Artificial Sequence
  synthetic
acagcc

SEQ ID NO: 281 - DNA, 6 bp, Artificial Sequence
  synthetic
agtccc

SEQ ID NO: 282 - DNA, 6 bp, Artificial Sequence
  synthetic
gtaccc

SEQ ID NO: 283 - DNA, 6 bp, Artificial Sequence
  synthetic
ctaccc

SEQ ID NO: 284 - DNA, 6 bp, Artificial Sequence
  synthetic
cgtacc

SEQ ID NO: 285 - DNA, 6 bp, Artificial Sequence
  synthetic
agtacc

SEQ ID NO: 286 - DNA, 6 bp, Artificial Sequence
  synthetic
cctacc

SEQ ID NO: 287 - DNA, 6 bp, Artificial Sequence
  synthetic
gatacc

SEQ ID NO: 288 - DNA, 6 bp, Artificial Sequence
  synthetic
aatacc

SEQ ID NO: 289 - DNA, 6 bp, Artificial Sequence
  synthetic
tggacc

SEQ ID NO: 290 - DNA, 6 bp, Artificial Sequence
  synthetic
gtaacc

SEQ ID NO: 291 - DNA, 6 bp, Artificial Sequence
  synthetic
tattac

SEQ ID NO: 292 - DNA, 6 bp, Artificial Sequence
  synthetic
cattac

SEQ ID NO: 293 - DNA, 6 bp, Artificial Sequence
  synthetic
ttgtac

SEQ ID NO: 294 - DNA, 6 bp, Artificial Sequence
  synthetic
tagtac

SEQ ID NO: 295 - DNA, 6 bp, Artificial Sequence
  synthetic
gagtac

SEQ ID NO: 296 - DNA, 6 bp, Artificial Sequence
  synthetic
aagtac

SEQ ID NO: 297 - DNA, 6 bp, Artificial Sequence
  synthetic
atctac

SEQ ID NO: 298 - DNA, 6 bp, Artificial Sequence
  synthetic
ccctac

SEQ ID NO: 299 - DNA, 6 bp, Artificial Sequence
  synthetic
ggatac

SEQ ID NO: 300 - DNA, 6 bp, Artificial Sequence
  synthetic
cgatac

SEQ ID NO: 301 - DNA, 6 bp, Artificial Sequence
  synthetic
agatac

SEQ ID NO: 302 - DNA, 6 bp, Artificial Sequence
  synthetic
gcatac

SEQ ID NO: 303 - DNA, 6 bp, Artificial Sequence
  synthetic
gaatac

SEQ ID NO: 304 - DNA, 6 bp, Artificial Sequence
  synthetic
aaatac

SEQ ID NO: 305 - DNA, 6 bp, Artificial Sequence
  synthetic
agtgac

SEQ ID NO: 306 - DNA, 6 bp, Artificial Sequence
  synthetic
cctgac

SEQ ID NO: 307 - DNA, 6 bp, Artificial Sequence
  synthetic
catgac

SEQ ID NO: 308 - DNA, 6 bp, Artificial Sequence
  synthetic
tgggac

SEQ ID NO: 309 - DNA, 6 bp, Artificial Sequence
  synthetic
gtcgac

SEQ ID NO: 310 - DNA, 6 bp, Artificial Sequence
  synthetic
atcgac

SEQ ID NO: 311 - DNA, 6 bp, Artificial Sequence
  synthetic
tgcgac

SEQ ID NO: 312 - DNA, 6 bp, Artificial Sequence
  synthetic
aacgac

SEQ ID NO: 313 - DNA, 6 bp, Artificial Sequence
  synthetic
ctagac

SEQ ID NO: 314 - DNA, 6 bp, Artificial Sequence
  synthetic
taagac

SEQ ID NO: 315 - DNA, 6 bp, Artificial Sequence
  synthetic
tcgcac

SEQ ID NO: 316 - DNA, 6 bp, Artificial Sequence
  synthetic
aaccac

SEQ ID NO: 317 - DNA, 6 bp, Artificial Sequence
  synthetic
agacac

SEQ ID NO: 318 - DNA, 6 bp, Artificial Sequence
  synthetic
gttaac

SEQ ID NO: 319 - DNA, 6 bp, Artificial Sequence
  synthetic
tctaac

SEQ ID NO: 320 - DNA, 6 bp, Artificial Sequence
  synthetic
gctaac

SEQ ID NO: 321 - DNA, 6 bp, Artificial Sequence
  synthetic
tataac

SEQ ID NO: 322 - DNA, 6 bp, Artificial Sequence
  synthetic
ccgaac

SEQ ID NO: 323 - DNA, 6 bp, Artificial Sequence
  synthetic
cgcaac

SEQ ID NO: 324 - DNA, 6 bp, Artificial Sequence
  synthetic
cacaac

SEQ ID NO: 325 - DNA, 6 bp, Artificial Sequence
  synthetic
ataaac

SEQ ID NO: 326 - DNA, 6 bp, Artificial Sequence
  synthetic
tgaaac

SEQ ID NO: 327 - DNA, 6 bp, Artificial Sequence
  synthetic
gcaaac

SEQ ID NO: 328 - DNA, 6 bp, Artificial Sequence
  synthetic
atttta

SEQ ID NO: 329 - DNA, 6 bp, Artificial Sequence
  synthetic
tgttta

SEQ ID NO: 330 - DNA, 6 bp, Artificial Sequence
  synthetic
acgtta

SEQ ID NO: 331 - DNA, 6 bp, Artificial Sequence
  synthetic
gagtta

SEQ ID NO: 332 - DNA, 6 bp, Artificial Sequence
  synthetic
aactta

SEQ ID NO: 333 - DNA, 6 bp, Artificial Sequence
  synthetic
agatta

SEQ ID NO: 334 - DNA, 6 bp, Artificial Sequence
  synthetic
gttgta

SEQ ID NO: 335 - DNA, 6 bp, Artificial Sequence
  synthetic
cttgta

SEQ ID NO: 336 - DNA, 6 bp, Artificial Sequence
  synthetic
cgtgta

SEQ ID NO: 337 - DNA, 6 bp, Artificial Sequence
  synthetic
tatgta

SEQ ID NO: 338 - DNA, 6 bp, Artificial Sequence
  synthetic
gatgta

SEQ ID NO: 339 - DNA, 6 bp, Artificial Sequence
  synthetic
gaggta

SEQ ID NO: 340 - DNA, 6 bp, Artificial Sequence
  synthetic
agcgta

SEQ ID NO: 341 - DNA, 6 bp, Artificial Sequence
  synthetic
cccgta

SEQ ID NO: 342 - DNA, 6 bp, Artificial Sequence
  synthetic
accgta

SEQ ID NO: 343 - DNA, 6 bp, Artificial Sequence
  synthetic
gacgta

SEQ ID NO: 344 - DNA, 6 bp, Artificial Sequence
  synthetic
aacgta

SEQ ID NO: 345 - DNA, 6 bp, Artificial Sequence
  synthetic
ctagta

SEQ ID NO: 346 - DNA, 6 bp, Artificial Sequence
  synthetic
ggagta

SEQ ID NO: 347 - DNA, 6 bp, Artificial Sequence
  synthetic
cgagta

SEQ ID NO: 348 - DNA, 6 bp, Artificial Sequence
  synthetic
acagta

SEQ ID NO: 349 - DNA, 6 bp, Artificial Sequence
  synthetic
taagta

SEQ ID NO: 350 - DNA, 6 bp, Artificial Sequence
  synthetic
gtgcta

SEQ ID NO: 351 - DNA, 6 bp, Artificial Sequence
  synthetic
gcgcta

SEQ ID NO: 352 - DNA, 6 bp, Artificial Sequence
  synthetic
aagcta

SEQ ID NO: 353 - DNA, 6 bp, Artificial Sequence
  synthetic
atccta

SEQ ID NO: 354 - DNA, 6 bp, Artificial Sequence
  synthetic
tgccta

SEQ ID NO: 355 - DNA, 6 bp, Artificial Sequence
  synthetic
gcacta

SEQ ID NO: 356 - DNA, 6 bp, Artificial Sequence
  synthetic
acacta

SEQ ID NO: 357 - DNA, 6 bp, Artificial Sequence
  synthetic
tttata

SEQ ID NO: 358 - DNA, 6 bp, Artificial Sequence
  synthetic
attata

SEQ ID NO: 359 - DNA, 6 bp, Artificial Sequence
  synthetic
tgtata

SEQ ID NO: 360 - DNA, 6 bp, Artificial Sequence
  synthetic
cgtata

SEQ ID NO: 361 - DNA, 6 bp, Artificial Sequence
  synthetic
gatata

SEQ ID NO: 362 - DNA, 6 bp, Artificial Sequence
  synthetic
catata

SEQ ID NO: 363 - DNA, 6 bp, Artificial Sequence
  synthetic
aggata

SEQ ID NO: 364 - DNA, 6 bp, Artificial Sequence
  synthetic
tcgata

SEQ ID NO: 365 - DNA, 6 bp, Artificial Sequence
  synthetic
gcgata

SEQ ID NO: 366 - DNA, 6 bp, Artificial Sequence
  synthetic
ccgata

SEQ ID NO: 367 - DNA, 6 bp, Artificial Sequence
  synthetic
acgata

SEQ ID NO: 368 - DNA, 6 bp, Artificial Sequence
  synthetic
gagata

SEQ ID NO: 369 - DNA, 6 bp, Artificial Sequence
  synthetic
aagata

SEQ ID NO: 370 - DNA, 6 bp, Artificial Sequence
  synthetic
ctcata

SEQ ID NO: 371 - DNA, 6 bp, Artificial Sequence
  synthetic
atcata

SEQ ID NO: 372 - DNA, 6 bp, Artificial Sequence
  synthetic
tgcata

SEQ ID NO: 373 - DNA, 6 bp, Artificial Sequence
  synthetic
cgcata

SEQ ID NO: 374 - DNA, 6 bp, Artificial Sequence
  synthetic
gacata

SEQ ID NO: 375 - DNA, 6 bp, Artificial Sequence
  synthetic
aacata

SEQ ID NO: 376 - DNA, 6 bp, Artificial Sequence
  synthetic
cgaata

SEQ ID NO: 377 - DNA, 6 bp, Artificial Sequence
  synthetic
ccaata

SEQ ID NO: 378 - DNA, 6 bp, Artificial Sequence
  synthetic
acaata

SEQ ID NO: 379 - DNA, 6 bp, Artificial Sequence
  synthetic
taaata

SEQ ID NO: 380 - DNA, 6 bp, Artificial Sequence
  synthetic
caaata

SEQ ID NO: 381 - DNA, 6 bp, Artificial Sequence
  synthetic
gattga

SEQ ID NO: 382 - DNA, 6 bp, Artificial Sequence
  synthetic
atgtga

SEQ ID NO: 383 - DNA, 6 bp, Artificial Sequence
  synthetic
cggtga

SEQ ID NO: 384 - DNA, 6 bp, Artificial Sequence
  synthetic
ccgtga

SEQ ID NO: 385 - DNA, 6 bp, Artificial Sequence
  synthetic
acgtga

SEQ ID NO: 386 - DNA, 6 bp, Artificial Sequence
  synthetic
gagtga

SEQ ID NO: 387 - DNA, 6 bp, Artificial Sequence
  synthetic
acctga

SEQ ID NO: 388 - DNA, 6 bp, Artificial Sequence
  synthetic
cactga

SEQ ID NO: 389 - DNA, 6 bp, Artificial Sequence
  synthetic
ggatga

SEQ ID NO: 390 - DNA, 6 bp, Artificial Sequence
  synthetic
cgatga

SEQ ID NO: 391 - DNA, 6 bp, Artificial Sequence
  synthetic
tcatga

SEQ ID NO: 392 - DNA, 6 bp, Artificial Sequence
  synthetic
gcatga

SEQ ID NO: 393 - DNA, 6 bp, Artificial Sequence
  synthetic
acatga

SEQ ID NO: 394 - DNA, 6 bp, Artificial Sequence
  synthetic
gaatga

SEQ ID NO: 395 - DNA, 6 bp, Artificial Sequence
  synthetic
tgtgga

SEQ ID NO: 396 - DNA, 6 bp, Artificial Sequence
  synthetic
ctggga

SEQ ID NO: 397 - DNA, 6 bp, Artificial Sequence
  synthetic
attcga

SEQ ID NO: 398 - DNA, 6 bp, Artificial Sequence
  synthetic
cgtcga

SEQ ID NO: 399 - DNA, 6 bp, Artificial Sequence
  synthetic
agtcga

SEQ ID NO: 400 - DNA, 6 bp, Artificial Sequence
  synthetic
gctcga

SEQ ID NO: 401 - DNA, 6 bp, Artificial Sequence
  synthetic
actcga

SEQ ID NO: 402 - DNA, 6 bp, Artificial Sequence
  synthetic
gatcga

SEQ ID NO: 403 - DNA, 6 bp, Artificial Sequence
  synthetic
ttgcga

SEQ ID NO: 404 - DNA, 6 bp, Artificial Sequence
  synthetic
atgcga

SEQ ID NO: 405 - DNA, 6 bp, Artificial Sequence
  synthetic
acgcga

SEQ ID NO: 406 - DNA, 6 bp, Artificial Sequence
  synthetic
gtccga

SEQ ID NO: 407 - DNA, 6 bp, Artificial Sequence
  synthetic
cgacga

SEQ ID NO: 408 - DNA, 6 bp, Artificial Sequence
  synthetic
agacga

SEQ ID NO: 409 - DNA, 6 bp, Artificial Sequence
  synthetic
acacga

SEQ ID NO: 410 - DNA, 6 bp, Artificial Sequence
  synthetic
taacga

SEQ ID NO: 411 - DNA, 6 bp, Artificial Sequence
  synthetic
gaacga

SEQ ID NO: 412 - DNA, 6 bp, Artificial Sequence
  synthetic
caacga

SEQ ID NO: 413 - DNA, 6 bp, Artificial Sequence
  synthetic
cgtaga

SEQ ID NO: 414 - DNA, 6 bp, Artificial Sequence
  synthetic
cctaga

SEQ ID NO: 415 - DNA, 6 bp, Artificial Sequence
  synthetic
tataga

SEQ ID NO: 416 - DNA, 6 bp, Artificial Sequence
  synthetic
gtgaga

SEQ ID NO: 417 - DNA, 6 bp, Artificial Sequence
  synthetic
atgaga

SEQ ID NO: 418 - DNA, 6 bp, Artificial Sequence
  synthetic
acgaga

SEQ ID NO: 419 - DNA, 6 bp, Artificial Sequence
  synthetic
tagaga

SEQ ID NO: 420 - DNA, 6 bp, Artificial Sequence
  synthetic
cagaga

SEQ ID NO: 421 - DNA, 6 bp, Artificial Sequence
  synthetic
cgcaga

SEQ ID NO: 422 - DNA, 6 bp, Artificial Sequence
  synthetic
aacaga

SEQ ID NO: 423 - DNA, 6 bp, Artificial Sequence
  synthetic
ataaga

SEQ ID NO: 424 - DNA, 6 bp, Artificial Sequence
  synthetic
cgaaga

SEQ ID NO: 425 - DNA, 6 bp, Artificial Sequence
  synthetic
acaaga

SEQ ID NO: 426 - DNA, 6 bp, Artificial Sequence
  synthetic
taaaga

SEQ ID NO: 427 - DNA, 6 bp, Artificial Sequence
  synthetic
gattca

SEQ ID NO: 428 - DNA, 6 bp, Artificial Sequence
  synthetic
ccctca

SEQ ID NO: 429 - DNA, 6 bp, Artificial Sequence
  synthetic
tactca

SEQ ID NO: 430 - DNA, 6 bp, Artificial Sequence
  synthetic
gtatca

SEQ ID NO: 431 - DNA, 6 bp, Artificial Sequence
  synthetic
tgatca

SEQ ID NO: 432 - DNA, 6 bp, Artificial Sequence
  synthetic
caatca

SEQ ID NO: 433 - DNA, 6 bp, Artificial Sequence
  synthetic
gttgca

SEQ ID NO: 434 - DNA, 6 bp, Artificial Sequence
  synthetic
tgtgca

SEQ ID NO: 435 - DNA, 6 bp, Artificial Sequence
  synthetic
ccggca

SEQ ID NO: 436 - DNA, 6 bp, Artificial Sequence
  synthetic
gtcgca

SEQ ID NO: 437 - DNA, 6 bp, Artificial Sequence
  synthetic
tgcgca

SEQ ID NO: 438 - DNA, 6 bp, Artificial Sequence
  synthetic
agcgca

SEQ ID NO: 439 - DNA, 6 bp, Artificial Sequence
  synthetic
tacgca

SEQ ID NO: 440 - DNA, 6 bp, Artificial Sequence
  synthetic
gtagca

SEQ ID NO: 441 - DNA, 6 bp, Artificial Sequence
  synthetic
atagca

SEQ ID NO: 442 - DNA, 6 bp, Artificial Sequence
  synthetic
ggagca

SEQ ID NO: 443 - DNA, 6 bp, Artificial Sequence
  synthetic
aaagca

SEQ ID NO: 444 - DNA, 6 bp, Artificial Sequence
  synthetic
gtccca

SEQ ID NO: 445 - DNA, 6 bp, Artificial Sequence
  synthetic
gtacca

SEQ ID NO: 446 - DNA, 6 bp, Artificial Sequence
  synthetic
atacca

SEQ ID NO: 447 - DNA, 6 bp, Artificial Sequence
  synthetic
cttaca

SEQ ID NO: 448 - DNA, 6 bp, Artificial Sequence
  synthetic
ggtaca

SEQ ID NO: 449 - DNA, 6 bp, Artificial Sequence
  synthetic
actaca

SEQ ID NO: 450 - DNA, 6 bp, Artificial Sequence
  synthetic
tataca

SEQ ID NO: 451 - DNA, 6 bp, Artificial Sequence
  synthetic
gataca

SEQ ID NO: 452 - DNA, 6 bp, Artificial Sequence
  synthetic
aataca

SEQ ID NO: 453 - DNA, 6 bp, Artificial Sequence
  synthetic
gtgaca

SEQ ID NO: 454 - DNA, 6 bp, Artificial Sequence
  synthetic
atgaca

SEQ ID NO: 455 - DNA, 6 bp, Artificial Sequence
  synthetic
tcgaca

SEQ ID NO: 456 - DNA, 6 bp, Artificial Sequence
  synthetic
gcgaca

SEQ ID NO: 457 - DNA, 6 bp, Artificial Sequence
  synthetic
acgaca

SEQ ID NO: 458 - DNA, 6 bp, Artificial Sequence
  synthetic
aagaca

SEQ ID NO: 459 - DNA, 6 bp, Artificial Sequence
  synthetic
tgcaca

SEQ ID NO: 460 - DNA, 6 bp, Artificial Sequence
  synthetic
gacaca

SEQ ID NO: 461 - DNA, 6 bp, Artificial Sequence
  synthetic
ttaaca

SEQ ID NO: 462 - DNA, 6 bp, Artificial Sequence
  synthetic
cgaaca

SEQ ID NO: 463 - DNA, 6 bp, Artificial Sequence
  synthetic
caaaca

SEQ ID NO: 464 - DNA, 6 bp, Artificial Sequence
  synthetic
gtttaa

SEQ ID NO: 465 - DNA, 6 bp, Artificial Sequence
  synthetic
tattaa

SEQ ID NO: 466 - DNA, 6 bp, Artificial Sequence
  synthetic
ttgtaa

SEQ ID NO: 467 - DNA, 6 bp, Artificial Sequence
  synthetic
atgtaa

SEQ ID NO: 468 - DNA, 6 bp, Artificial Sequence
  synthetic
cggtaa

SEQ ID NO: 469 - DNA, 6 bp, Artificial Sequence
  synthetic
aggtaa

SEQ ID NO: 470 - DNA, 6 bp, Artificial Sequence
  synthetic
ccgtaa

SEQ ID NO: 471 - DNA, 6 bp, Artificial Sequence
  synthetic
acgtaa

SEQ ID NO: 472 - DNA, 6 bp, Artificial Sequence
  synthetic
gagtaa

SEQ ID NO: 473 - DNA, 6 bp, Artificial Sequence
  synthetic
cgctaa

SEQ ID NO: 474 - DNA, 6 bp, Artificial Sequence
  synthetic
gcctaa

SEQ ID NO: 475 - DNA, 6 bp, Artificial Sequence
  synthetic
ccctaa

SEQ ID NO: 476 - DNA, 6 bp, Artificial Sequence
  synthetic
cgataa

SEQ ID NO: 477 - DNA, 6 bp, Artificial Sequence
  synthetic
agataa

SEQ ID NO: 478 - DNA, 6 bp, Artificial Sequence
  synthetic
gcataa

SEQ ID NO: 479 - DNA, 6 bp, Artificial Sequence
  synthetic
acataa

SEQ ID NO: 480 - DNA, 6 bp, Artificial Sequence
  synthetic
caataa

SEQ ID NO: 481 - DNA, 6 bp, Artificial Sequence
  synthetic
cgtgaa

SEQ ID NO: 482 - DNA, 6 bp, Artificial Sequence
  synthetic
gatgaa

SEQ ID NO: 483 - DNA, 6 bp, Artificial Sequence
  synthetic
catgaa

SEQ ID NO: 484 - DNA, 6 bp, Artificial Sequence
  synthetic
ttggaa

SEQ ID NO: 485 - DNA, 6 bp, Artificial Sequence
  synthetic
tgcgaa

SEQ ID NO: 486 - DNA, 6 bp, Artificial Sequence
  synthetic
agcgaa

SEQ ID NO: 487 - DNA, 6 bp, Artificial Sequence
  synthetic
ttagaa

SEQ ID NO: 488 - DNA, 6 bp, Artificial Sequence
  synthetic
cctcaa

SEQ ID NO: 489 - DNA, 6 bp, Artificial Sequence
  synthetic
catcaa

SEQ ID NO: 490 - DNA, 6 bp, Artificial Sequence
  synthetic
ctgcaa

SEQ ID NO: 491 - DNA, 6 bp, Artificial Sequence
  synthetic
atgcaa

SEQ ID NO: 492 - DNA, 6 bp, Artificial Sequence
  synthetic
cggcaa

SEQ ID NO: 493 - DNA, 6 bp, Artificial Sequence
  synthetic
tcgcaa

SEQ ID NO: 494 - DNA, 6 bp, Artificial Sequence
  synthetic
ccgcaa

SEQ ID NO: 495 - DNA, 6 bp, Artificial Sequence
  synthetic
tagcaa

SEQ ID NO: 496 - DNA, 6 bp, Artificial Sequence
  synthetic
atacaa

SEQ ID NO: 497 - DNA, 6 bp, Artificial Sequence
  synthetic
tgacaa

SEQ ID NO: 498 - DNA, 6 bp, Artificial Sequence
  synthetic
cgacaa

SEQ ID NO: 499 - DNA, 6 bp, Artificial Sequence
  synthetic
gcacaa

SEQ ID NO: 500 - DNA, 6 bp, Artificial Sequence
  synthetic
acacaa

SEQ ID NO: 501 - DNA, 6 bp, Artificial Sequence
  synthetic
taacaa

SEQ ID NO: 502 - DNA, 6 bp, Artificial Sequence
  synthetic
aaacaa

SEQ ID NO: 503 - DNA, 6 bp, Artificial Sequence
  synthetic
tgtaaa

SEQ ID NO: 504 - DNA, 6 bp, Artificial Sequence
  synthetic
cctaaa

SEQ ID NO: 505 - DNA, 6 bp, Artificial Sequence
  synthetic
cataaa

SEQ ID NO: 506 - DNA, 6 bp, Artificial Sequence
  synthetic
gcgaaa

SEQ ID NO: 507 - DNA, 6 bp, Artificial Sequence
  synthetic
cgcaaa

SEQ ID NO: 508 - DNA, 6 bp, Artificial Sequence
  synthetic
ggaaaa

SEQ ID NO: 509 - DNA, 6 bp, Artificial Sequence
  synthetic
gcaaaa

SEQ ID NO: 510 - DNA, 6 bp, Artificial Sequence
  synthetic
taaaaa

SEQ ID NO: 511 - DNA, 6 bp, Artificial Sequence
  synthetic
acattt

SEQ ID NO: 512 - DNA, 6 bp, Artificial Sequence
  synthetic
tctgtt

SEQ ID NO: 513 - DNA, 6 bp, Artificial Sequence
  synthetic
ttgctt

SEQ ID NO: 514 - DNA, 6 bp, Artificial Sequence
  synthetic
gacctt

SEQ ID NO: 515 - DNA, 6 bp, Artificial Sequence
  synthetic
gaactt

SEQ ID NO: 516 - DNA, 6 bp, Artificial Sequence
  synthetic
cacatt

SEQ ID NO: 517 - DNA, 6 bp, Artificial Sequence
  synthetic
atttgt

SEQ ID NO: 518 - DNA, 6 bp, Artificial Sequence
  synthetic
tggtgt

SEQ ID NO: 519 - DNA, 6 bp, Artificial Sequence
  synthetic
gagtgt

SEQ ID NO: 520 - DNA, 6 bp, Artificial Sequence
  synthetic
aagtgt

SEQ ID NO: 521 - DNA, 6 bp, Artificial Sequence
  synthetic
ctctgt

SEQ ID NO: 522 - DNA, 6 bp, Artificial Sequence
  synthetic
ttatgt

SEQ ID NO: 523 - DNA, 6 bp, Artificial Sequence
  synthetic
ctgggt

SEQ ID NO: 524 - DNA, 6 bp, Artificial Sequence
  synthetic
aagggt

SEQ ID NO: 525 - DNA, 6 bp, Artificial Sequence
  synthetic
tgtcgt

SEQ ID NO: 526 - DNA, 6 bp, Artificial Sequence
  synthetic
tggcgt

SEQ ID NO: 527 - DNA, 6 bp, Artificial Sequence
  synthetic
cagcgt

SEQ ID NO: 528 - DNA, 6 bp, Artificial Sequence
  synthetic
tgacgt

SEQ ID NO: 529 - DNA, 6 bp, Artificial Sequence
  synthetic
ctgagt

SEQ ID NO: 530 - DNA, 6 bp, Artificial Sequence
  synthetic
tgcagt

SEQ ID NO: 531 - DNA, 6 bp, Artificial Sequence
  synthetic
ggcagt

SEQ ID NO: 532 - DNA, 6 bp, Artificial Sequence
  synthetic
ggaagt

SEQ ID NO: 533 - DNA, 6 bp, Artificial Sequence
  synthetic
acttct

SEQ ID NO: 534 - DNA, 6 bp, Artificial Sequence
  synthetic
gtgtct

SEQ ID NO: 535 - DNA, 6 bp, Artificial Sequence
  synthetic
tggtct

SEQ ID NO: 536 - DNA, 6 bp, Artificial Sequence
  synthetic
aggtct

SEQ ID NO: 537 - DNA, 6 bp, Artificial Sequence
  synthetic
gcgtct

SEQ ID NO: 538 - DNA, 6 bp, Artificial Sequence
  synthetic
cagtct

SEQ ID NO: 539 - DNA, 6 bp, Artificial Sequence
  synthetic
gcatct

SEQ ID NO: 540 - DNA, 6 bp, Artificial Sequence
  synthetic
gttgct

SEQ ID NO: 541 - DNA, 6 bp, Artificial Sequence
  synthetic
ggtgct

SEQ ID NO: 542 - DNA, 6 bp, Artificial Sequence
  synthetic
acggct

SEQ ID NO: 543 - DNA, 6 bp, Artificial Sequence
  synthetic
catcct

SEQ ID NO: 544 - DNA, 6 bp, Artificial Sequence
  synthetic
gagcct

SEQ ID NO: 545 - DNA, 6 bp, Artificial Sequence
  synthetic
cagcct

SEQ ID NO: 546 - DNA, 6 bp, Artificial Sequence
  synthetic
aagcct

SEQ ID NO: 547 - DNA, 6 bp, Artificial Sequence
  synthetic
taccct

SEQ ID NO: 548 - DNA, 6 bp, Artificial Sequence
  synthetic
gatact

SEQ ID NO: 549 - DNA, 6 bp, Artificial Sequence
  synthetic
accact

SEQ ID NO: 550 - DNA, 6 bp, Artificial Sequence
  synthetic
ttttat

SEQ ID NO: 551 - DNA, 6 bp, Artificial Sequence
  synthetic
atttat

SEQ ID NO: 552 - DNA, 6 bp, Artificial Sequence
  synthetic
tcttat

SEQ ID NO: 553 - DNA, 6 bp, Artificial Sequence
  synthetic
ttgtat

SEQ ID NO: 554 - DNA, 6 bp, Artificial Sequence
  synthetic
attgat

SEQ ID NO: 555 - DNA, 6 bp, Artificial Sequence
  synthetic
tgtgat

SEQ ID NO: 556 - DNA, 6 bp, Artificial Sequence
  synthetic
catgat

SEQ ID NO: 557 - DNA, 6 bp, Artificial Sequence
  synthetic
ccagat

SEQ ID NO: 558 - DNA, 6 bp, Artificial Sequence
  synthetic
gatcat

SEQ ID NO: 559 - DNA, 6 bp, Artificial Sequence
  synthetic
tggcat

SEQ ID NO: 560 - DNA, 6 bp, Artificial Sequence
  synthetic
cagcat

SEQ ID NO: 561 - DNA, 6 bp, Artificial Sequence
  synthetic
gtccat

SEQ ID NO: 562 - DNA, 6 bp, Artificial Sequence
  synthetic
tgccat

SEQ ID NO: 563 - DNA, 6 bp, Artificial Sequence
  synthetic
gaacat

SEQ ID NO: 564 - DNA, 6 bp, Artificial Sequence
  synthetic
agtaat

SEQ ID NO: 565 - DNA, 6 bp, Artificial Sequence
  synthetic
gtgaat

SEQ ID NO: 566 - DNA, 6 bp, Artificial Sequence
  synthetic
ctgaat

SEQ ID NO: 567 - DNA, 6 bp, Artificial Sequence
  synthetic
cagaat

SEQ ID NO: 568 - DNA, 6 bp, Artificial Sequence
  synthetic
tgaaat

SEQ ID NO: 569 - DNA, 6 bp, Artificial Sequence
  synthetic
gcgttg

SEQ ID NO: 570 - DNA, 6 bp, Artificial Sequence
  synthetic
acgttg

SEQ ID NO: 571 - DNA, 6 bp, Artificial Sequence
  synthetic
cagttg

SEQ ID NO: 572 - DNA, 6 bp, Artificial Sequence
  synthetic
gccttg

SEQ ID NO: 573 - DNA, 6 bp, Artificial Sequence
  synthetic
gttgtg

SEQ ID NO: 574 - DNA, 6 bp, Artificial Sequence
  synthetic
agtgtg

SEQ ID NO: 575 - DNA, 6 bp, Artificial Sequence
  synthetic
atggtg

SEQ ID NO: 576 - DNA, 6 bp, Artificial Sequence
  synthetic
acggtg

SEQ ID NO: 577 - DNA, 6 bp, Artificial Sequence
  synthetic
agcgtg

SEQ ID NO: 578 - DNA, 6 bp, Artificial Sequence
  synthetic
gcagtg

SEQ ID NO: 579 - DNA, 6 bp, Artificial Sequence
  synthetic
gaagtg

SEQ ID NO: 580 - DNA, 6 bp, Artificial Sequence
  synthetic
agtctg

SEQ ID NO: 581 - DNA, 6 bp, Artificial Sequence
  synthetic
tctctg

SEQ ID NO: 582 - DNA, 6 bp, Artificial Sequence
  synthetic
agcctg

SEQ ID NO: 583 - DNA, 6 bp, Artificial Sequence
  synthetic
ccactg

SEQ ID NO: 584 - DNA, 6 bp, Artificial Sequence
  synthetic
acactg

SEQ ID NO: 585 - DNA, 6 bp, Artificial Sequence
  synthetic
atgatg

SEQ ID NO: 586 - DNA, 6 bp, Artificial Sequence
  synthetic
tcaatg

SEQ ID NO: 587 - DNA, 6 bp, Artificial Sequence
  synthetic
ttgtgg

SEQ ID NO: 588 - DNA, 6 bp, Artificial Sequence
  synthetic
atctgg

SEQ ID NO: 589 - DNA, 6 bp, Artificial Sequence
  synthetic
tgatgg

SEQ ID NO: 590 - DNA, 6 bp, Artificial Sequence
  synthetic
gatggg

SEQ ID NO: 591 - DNA, 6 bp, Artificial Sequence
  synthetic
cagggg

SEQ ID NO: 592 - DNA, 6 bp, Artificial Sequence
  synthetic
tgcggg

SEQ ID NO: 593 - DNA, 6 bp, Artificial Sequence
  synthetic
tgtcgg

SEQ ID NO: 594 - DNA, 6 bp, Artificial Sequence
  synthetic
aaacgg

SEQ ID NO: 595 - DNA, 6 bp, Artificial Sequence
  synthetic
attagg

SEQ ID NO: 596 - DNA, 6 bp, Artificial Sequence
  synthetic
tctagg

SEQ ID NO: 597 - DNA, 6 bp, Artificial Sequence
  synthetic
cacagg

SEQ ID NO: 598 - DNA, 6 bp, Artificial Sequence
  synthetic
atgtcg

SEQ ID NO: 599 - DNA, 6 bp, Artificial Sequence
  synthetic
aactcg

SEQ ID NO: 600 - DNA, 6 bp, Artificial Sequence
  synthetic
gttgcg

SEQ ID NO: 601 - DNA, 6 bp, Artificial Sequence
  synthetic
tgtgcg

SEQ ID NO: 602 - DNA, 6 bp, Artificial Sequence
  synthetic
agtgcg

SEQ ID NO: 603 - DNA, 6 bp, Artificial Sequence
  synthetic
acagcg

SEQ ID NO: 604 - DNA, 6 bp, Artificial Sequence
  synthetic
ttgacg

SEQ ID NO: 605 - DNA, 6 bp, Artificial Sequence
  synthetic
agcacg

SEQ ID NO: 606 - DNA, 6 bp, Artificial Sequence
  synthetic
accacg

SEQ ID NO: 607 - DNA, 6 bp, Artificial Sequence
  synthetic
gtaacg

SEQ ID NO: 608 - DNA, 6 bp, Artificial Sequence
  synthetic
acctag

SEQ ID NO: 609 - DNA, 6 bp, Artificial Sequence
  synthetic
tgtgag

SEQ ID NO: 610 - DNA, 6 bp, Artificial Sequence
  synthetic
catgag

SEQ ID NO: 611 - DNA, 6 bp, Artificial Sequence
  synthetic
caggag

SEQ ID NO: 612 - DNA, 6 bp, Artificial Sequence
  synthetic
aaggag

SEQ ID NO: 613 - DNA, 6 bp, Artificial Sequence
  synthetic
gcagag

SEQ ID NO: 614 - DNA, 6 bp, Artificial Sequence
  synthetic
gctcag

SEQ ID NO: 615 - DNA, 6 bp, Artificial Sequence
  synthetic
tatcag

SEQ ID NO: 616 - DNA, 6 bp, Artificial Sequence
  synthetic
ttgcag

SEQ ID NO: 617 - DNA, 6 bp, Artificial Sequence
  synthetic
aggcag

SEQ ID NO: 618 - DNA, 6 bp, Artificial Sequence
  synthetic
tagcag

SEQ ID NO: 619 - DNA, 6 bp, Artificial Sequence
  synthetic
cagcag

SEQ ID NO: 620 - DNA, 6 bp, Artificial Sequence
  synthetic
gaccag

SEQ ID NO: 621 - DNA, 6 bp, Artificial Sequence
  synthetic
acacag

SEQ ID NO: 622 - DNA, 6 bp, Artificial Sequence
  synthetic
ctcaag

SEQ ID NO: 623 - DNA, 6 bp, Artificial Sequence
  synthetic
tgcaag

SEQ ID NO: 624 - DNA, 6 bp, Artificial Sequence
  synthetic
ataaag

SEQ ID NO: 625 - DNA, 6 bp, Artificial Sequence
  synthetic
tgaaag

SEQ ID NO: 626 - DNA, 6 bp, Artificial Sequence
  synthetic
ggtgtc

SEQ ID NO: 627 - DNA, 6 bp, Artificial Sequence
  synthetic
tatgtc

SEQ ID NO: 628 - DNA, 6 bp, Artificial Sequence
  synthetic
taggtc

SEQ ID NO: 629 - DNA, 6 bp, Artificial Sequence
  synthetic
ggcgtc

SEQ ID NO: 630 - DNA, 6 bp, Artificial Sequence
  synthetic
ggagtc

SEQ ID NO: 631 - DNA, 6 bp, Artificial Sequence
  synthetic
gcagtc

SEQ ID NO: 632 - DNA, 6 bp, Artificial Sequence
  synthetic
gatctc

SEQ ID NO: 633 - DNA, 6 bp, Artificial Sequence
  synthetic
atgctc

SEQ ID NO: 634 - DNA, 6 bp, Artificial Sequence
  synthetic
cctatc

SEQ ID NO: 635 - DNA, 6 bp, Artificial Sequence
  synthetic
aatatc

SEQ ID NO: 636 - DNA, 6 bp, Artificial Sequence
  synthetic
tgcatc

SEQ ID NO: 637 - DNA, 6 bp, Artificial Sequence
  synthetic
agaatc

SEQ ID NO: 638 - DNA, 6 bp, Artificial Sequence
  synthetic
ggttgc

SEQ ID NO: 639 - DNA, 6 bp, Artificial Sequence
  synthetic
cgttgc

SEQ ID NO: 640 - DNA, 6 bp, Artificial Sequence
  synthetic
agttgc

SEQ ID NO: 641 - DNA, 6 bp, Artificial Sequence
  synthetic
ttgtgc

SEQ ID NO: 642 - DNA, 6 bp, Artificial Sequence
  synthetic
atgtgc

SEQ ID NO: 643 - DNA, 6 bp, Artificial Sequence
  synthetic
aggtgc

SEQ ID NO: 644 - DNA, 6 bp, Artificial Sequence
  synthetic
cagtgc

SEQ ID NO: 645 - DNA, 6 bp, Artificial Sequence
  synthetic
agatgc

SEQ ID NO: 646 - DNA, 6 bp, Artificial Sequence
  synthetic
tatggc

SEQ ID NO: 647 - DNA, 6 bp, Artificial Sequence
  synthetic
gtgagc

SEQ ID NO: 648 - DNA, 6 bp, Artificial Sequence
  synthetic
ggcagc

SEQ ID NO: 649 - DNA, 6 bp, Artificial Sequence
  synthetic
agcagc

SEQ ID NO: 650 - DNA, 6 bp, Artificial Sequence
  synthetic
aacagc

SEQ ID NO: 651 - DNA, 6 bp, Artificial Sequence
  synthetic
cgaagc

SEQ ID NO: 652 - DNA, 6 bp, Artificial Sequence
  synthetic
gaaagc

SEQ ID NO: 653 - DNA, 6 bp, Artificial Sequence
  synthetic
atttcc

SEQ ID NO: 654 - DNA, 6 bp, Artificial Sequence
  synthetic
atatcc

SEQ ID NO: 655 - DNA, 6 bp, Artificial Sequence
  synthetic
acatcc

SEQ ID NO: 656 - DNA, 6 bp, Artificial Sequence
  synthetic
gttgcc

SEQ ID NO: 657 - DNA, 6 bp, Artificial Sequence
  synthetic
attgcc

SEQ ID NO: 658 - DNA, 6 bp, Artificial Sequence
  synthetic
tgtgcc

SEQ ID NO: 659 - DNA, 6 bp, Artificial Sequence
  synthetic
agtgcc

SEQ ID NO: 660 - DNA, 6 bp, Artificial Sequence
  synthetic
tctgcc

SEQ ID NO: 661 - DNA, 6 bp, Artificial Sequence
  synthetic
ctggcc

SEQ ID NO: 662 - DNA, 6 bp, Artificial Sequence
  synthetic
caggcc

SEQ ID NO: 663 - DNA, 6 bp, Artificial Sequence
  synthetic
aaggcc

SEQ ID NO: 664 - DNA, 6 bp, Artificial Sequence
  synthetic
gaagcc

SEQ ID NO: 665 - DNA, 6 bp, Artificial Sequence
  synthetic
tacccc

SEQ ID NO: 666 - DNA, 6 bp, Artificial Sequence
  synthetic
catacc

SEQ ID NO: 667 - DNA, 6 bp, Artificial Sequence
  synthetic
tagacc

SEQ ID NO: 668 - DNA, 6 bp, Artificial Sequence
  synthetic
ataacc

SEQ ID NO: 669 - DNA, 6 bp, Artificial Sequence
  synthetic
tggtac

SEQ ID NO: 670 - DNA, 6 bp, Artificial Sequence
  synthetic
tgatac

SEQ ID NO: 671 - DNA, 6 bp, Artificial Sequence
  synthetic
gtagac

SEQ ID NO: 672 - DNA, 6 bp, Artificial Sequence
  synthetic
tcagac

SEQ ID NO: 673 - DNA, 6 bp, Artificial Sequence
  synthetic
attcac

SEQ ID NO: 674 - DNA, 6 bp, Artificial Sequence
  synthetic
tagcac

SEQ ID NO: 675 - DNA, 6 bp, Artificial Sequence
  synthetic
cagcac

SEQ ID NO: 676 - DNA, 6 bp, Artificial Sequence
  synthetic
gaccac

SEQ ID NO: 677 - DNA, 6 bp, Artificial Sequence
  synthetic
agtaac

SEQ ID NO: 678 - DNA, 6 bp, Artificial Sequence
  synthetic
gataac

SEQ ID NO: 679 - DNA, 6 bp, Artificial Sequence
  synthetic
caaaac

SEQ ID NO: 680 - DNA, 6 bp, Artificial Sequence
  synthetic
ttatta

SEQ ID NO: 681 - DNA, 6 bp, Artificial Sequence
  synthetic
gcagta

SEQ ID NO: 682 - DNA, 6 bp, Artificial Sequence
  synthetic
aatcta

SEQ ID NO: 683 - DNA, 6 bp, Artificial Sequence
  synthetic
agccta

SEQ ID NO: 684 - DNA, 6 bp, Artificial Sequence
  synthetic
gccata

SEQ ID NO: 685 - DNA, 6 bp, Artificial Sequence
  synthetic
cccata

SEQ ID NO: 686 - DNA, 6 bp, Artificial Sequence
  synthetic
gaaata

SEQ ID NO: 687 - DNA, 6 bp, Artificial Sequence
  synthetic
ctgtga

SEQ ID NO: 688 - DNA, 6 bp, Artificial Sequence
  synthetic
tagtga

SEQ ID NO: 689 - DNA, 6 bp, Artificial Sequence
  synthetic
ctctga

SEQ ID NO: 690 - DNA, 6 bp, Artificial Sequence
  synthetic
gcctga

SEQ ID NO: 691 - DNA, 6 bp, Artificial Sequence
  synthetic
ccatga

SEQ ID NO: 692 - DNA, 6 bp, Artificial Sequence
  synthetic
aaatga

SEQ ID NO: 693 - DNA, 6 bp, Artificial Sequence
  synthetic
gttgga

SEQ ID NO: 694 - DNA, 6 bp, Artificial Sequence
  synthetic
tctgga

SEQ ID NO: 695 - DNA, 6 bp, Artificial Sequence
  synthetic
acagga

SEQ ID NO: 696 - DNA, 6 bp, Artificial Sequence
  synthetic
caagga

SEQ ID NO: 697 - DNA, 6 bp, Artificial Sequence
  synthetic
ggtcga

SEQ ID NO: 698 - DNA, 6 bp, Artificial Sequence
  synthetic
taccga

SEQ ID NO: 699 - DNA, 6 bp, Artificial Sequence
  synthetic
caccga

SEQ ID NO: 700 - DNA, 6 bp, Artificial Sequence
  synthetic
ctgaga

SEQ ID NO: 701 - DNA, 6 bp, Artificial Sequence
  synthetic
agcaga

SEQ ID NO: 702 - DNA, 6 bp, Artificial Sequence
  synthetic
gacaga

SEQ ID NO: 703 - DNA, 6 bp, Artificial Sequence
  synthetic
agaaga

SEQ ID NO: 704 - DNA, 6 bp, Artificial Sequence
  synthetic
acttca

SEQ ID NO: 705 - DNA, 6 bp, Artificial Sequence
  synthetic
tattca

SEQ ID NO: 706 - DNA, 6 bp, Artificial Sequence
  synthetic
atgtca

SEQ ID NO: 707 - DNA, 6 bp, Artificial Sequence
  synthetic
cggtca

SEQ ID NO: 708 - DNA, 6 bp, Artificial Sequence
  synthetic
aagtca

SEQ ID NO: 709 - DNA, 6 bp, Artificial Sequence
  synthetic
atctca

SEQ ID NO: 710 - DNA, 6 bp, Artificial Sequence
  synthetic
cgctca

SEQ ID NO: 711 - DNA, 6 bp, Artificial Sequence
  synthetic
ttatca

SEQ ID NO: 712 - DNA, 6 bp, Artificial Sequence
  synthetic
gaatca

SEQ ID NO: 713 - DNA, 6 bp, Artificial Sequence
  synthetic
ggtgca

SEQ ID NO: 714 - DNA, 6 bp, Artificial Sequence
  synthetic
cctgca

SEQ ID NO: 715 - DNA, 6 bp, Artificial Sequence
  synthetic
gatgca

SEQ ID NO: 716 - DNA, 6 bp, Artificial Sequence
  synthetic
gtggca

SEQ ID NO: 717 - DNA, 6 bp, Artificial Sequence
  synthetic
acggca

SEQ ID NO: 718 - DNA, 6 bp, Artificial Sequence
  synthetic
ctagca

SEQ ID NO: 719 - DNA, 6 bp, Artificial Sequence
  synthetic
tcagca

SEQ ID NO: 720 - DNA, 6 bp, Artificial Sequence
  synthetic
ccagca

SEQ ID NO: 721 - DNA, 6 bp, Artificial Sequence
  synthetic
acagca

SEQ ID NO: 722 - DNA, 6 bp, Artificial Sequence
  synthetic
agtcca

SEQ ID NO: 723 - DNA, 6 bp, Artificial Sequence
  synthetic
actcca

SEQ ID NO: 724 - DNA, 6 bp, Artificial Sequence
  synthetic
ctgcca

SEQ ID NO: 725 - DNA, 6 bp, Artificial Sequence
  synthetic
tagcca

SEQ ID NO: 726 - DNA, 6 bp, Artificial Sequence
  synthetic
agacca

SEQ ID NO: 727 - DNA, 6 bp, Artificial Sequence
  synthetic
gtcaca

SEQ ID NO: 728 - DNA, 6 bp, Artificial Sequence
  synthetic
tccaca

SEQ ID NO: 729 - DNA, 6 bp, Artificial Sequence
  synthetic
cacaca

SEQ ID NO: 730 - DNA, 6 bp, Artificial Sequence
  synthetic
ataaca

SEQ ID NO: 731 - DNA, 6 bp, Artificial Sequence
  synthetic
gaaaca

SEQ ID NO: 732 - DNA, 6 bp, Artificial Sequence
  synthetic
cagtaa

SEQ ID NO: 733 - DNA, 6 bp, Artificial Sequence
  synthetic
aaataa

SEQ ID NO: 734 - DNA, 6 bp, Artificial Sequence
  synthetic
cctgaa

SEQ ID NO: 735 - DNA, 6 bp, Artificial Sequence
  synthetic
caggaa

SEQ ID NO: 736 - DNA, 6 bp, Artificial Sequence
  synthetic
gtcgaa

SEQ ID NO: 737 - DNA, 6 bp, Artificial Sequence
  synthetic
gccgaa

SEQ ID NO: 738 - DNA, 6 bp, Artificial Sequence
  synthetic
gaagaa

SEQ ID NO: 739 - DNA, 6 bp, Artificial Sequence
  synthetic
attcaa

SEQ ID NO: 740 - DNA, 6 bp, Artificial Sequence
  synthetic
tctcaa

SEQ ID NO: 741 - DNA, 6 bp, Artificial Sequence
  synthetic
actcaa

SEQ ID NO: 742 - DNA, 6 bp, Artificial Sequence
  synthetic
gtgcaa

SEQ ID NO: 743 - DNA, 6 bp, Artificial Sequence
  synthetic
tgccaa

SEQ ID NO: 744 - DNA, 6 bp, Artificial Sequence
  synthetic
gcccaa

SEQ ID NO: 745 - DNA, 6 bp, Artificial Sequence
  synthetic
ttgaaa

SEQ ID NO: 746 - DNA, 6 bp, Artificial Sequence
  synthetic
aggaaa

SEQ ID NO: 747 - DNA, 6 bp, Artificial Sequence
  synthetic
ctcaaa

SEQ ID NO: 748 - DNA, 6 bp, Artificial Sequence
  synthetic
agcaaa

SEQ ID NO: 749 - DNA, 6 bp, Artificial Sequence
  synthetic
gccaaa

SEQ ID NO: 750 - DNA, 6 bp, Artificial Sequence
  synthetic
aaacgt

SEQ ID NO: 751 - DNA, 6 bp, Artificial Sequence
  synthetic
aaccgg

SEQ ID NO: 752 - DNA, 6 bp, Artificial Sequence
  synthetic
aacgac

SEQ ID NO: 753 - DNA, 6 bp, Artificial Sequence
  synthetic
aacgta

SEQ ID NO: 754 - DNA, 6 bp, Artificial Sequence
  synthetic
aacgtt

SEQ ID NO: 755 - DNA, 6 bp, Artificial Sequence
  synthetic
aagaca

SEQ ID NO: 756 - DNA, 6 bp, Artificial Sequence
  synthetic
aagatc

SEQ ID NO: 757 - DNA, 6 bp, Artificial Sequence
  synthetic
aagggg

SEQ ID NO: 758 - DNA, 6 bp, Artificial Sequence
  synthetic
aagtag

SEQ ID NO: 759 - DNA, 6 bp, Artificial Sequence
  synthetic
aatacg

SEQ ID NO: 760 - DNA, 6 bp, Artificial Sequence
  synthetic
aatact

SEQ ID NO: 761 - DNA, 6 bp, Artificial Sequence
  synthetic
aatatg

SEQ ID NO: 762 - DNA, 6 bp, Artificial Sequence
  synthetic
aatcat

SEQ ID NO: 763 - DNA, 6 bp, Artificial Sequence
  synthetic
aatcgc

SEQ ID NO: 764 - DNA, 6 bp, Artificial Sequence
  synthetic
aatcgt

SEQ ID NO: 765 - DNA, 6 bp, Artificial Sequence
  synthetic
aatctg

SEQ ID NO: 766 - DNA, 6 bp, Artificial Sequence
  synthetic
aatgac

SEQ ID NO: 767 - DNA, 6 bp, Artificial Sequence
  synthetic
aatgct

SEQ ID NO: 768 - DNA, 6 bp, Artificial Sequence
  synthetic
aatggt

SEQ ID NO: 769 - DNA, 6 bp, Artificial Sequence
  synthetic
aattgc

SEQ ID NO: 770 - DNA, 6 bp, Artificial Sequence
  synthetic
aattgt

SEQ ID NO: 771 - DNA, 6 bp, Artificial Sequence
  synthetic
aatttt

SEQ ID NO: 772 - DNA, 6 bp, Artificial Sequence
  synthetic
acaaca

SEQ ID NO: 773 - DNA, 6 bp, Artificial Sequence
  synthetic
acaacg

SEQ ID NO: 774 - DNA, 6 bp, Artificial Sequence
  synthetic
acacgc

SEQ ID NO: 775 - DNA, 6 bp, Artificial Sequence
  synthetic
acagtc

SEQ ID NO: 776 - DNA, 6 bp, Artificial Sequence
  synthetic
acatac

SEQ ID NO: 777 - DNA, 6 bp, Artificial Sequence
  synthetic
acatag

SEQ ID NO: 778 - DNA, 6 bp, Artificial Sequence
  synthetic
acatcg

SEQ ID NO: 779 - DNA, 6 bp, Artificial Sequence
  synthetic
acatct

SEQ ID NO: 780 - DNA, 6 bp, Artificial Sequence
  synthetic
acatta

SEQ ID NO: 781 - DNA, 6 bp, Artificial Sequence
  synthetic
acattg

SEQ ID NO: 782 - DNA, 6 bp, Artificial Sequence
  synthetic
accagc

SEQ ID NO: 783 - DNA, 6 bp, Artificial Sequence
  synthetic
accgac

SEQ ID NO: 784 - DNA, 6 bp, Artificial Sequence
  synthetic
accggt

SEQ ID NO: 785 - DNA, 6 bp, Artificial Sequence
  synthetic
acgaag

SEQ ID NO: 786 - DNA, 6 bp, Artificial Sequence
  synthetic
acgacg

SEQ ID NO: 787 - DNA, 6 bp, Artificial Sequence
  synthetic
acgact

SEQ ID NO: 788 - DNA, 6 bp, Artificial Sequence
  synthetic
acgcaa

SEQ ID NO: 789 - DNA, 6 bp, Artificial Sequence
  synthetic
acgcgg

SEQ ID NO: 790 - DNA, 6 bp, Artificial Sequence
  synthetic
acgcgt

SEQ ID NO: 791 - DNA, 6 bp, Artificial Sequence
  synthetic
acggag

SEQ ID NO: 792 - DNA, 6 bp, Artificial Sequence
  synthetic
acggat

SEQ ID NO: 793 - DNA, 6 bp, Artificial Sequence
  synthetic
acggtt

SEQ ID NO: 794 - DNA, 6 bp, Artificial Sequence
  synthetic
acgtac

SEQ ID NO: 795 - DNA, 6 bp, Artificial Sequence
  synthetic
acgtat

SEQ ID NO: 796 - DNA, 6 bp, Artificial Sequence
  synthetic
acgtcc

SEQ ID NO: 797 - DNA, 6 bp, Artificial Sequence
  synthetic
acgttg

SEQ ID NO: 798 - DNA, 6 bp, Artificial Sequence
  synthetic
acgttt

SEQ ID NO: 799 - DNA, 6 bp, Artificial Sequence
  synthetic
actaca

SEQ ID NO: 800 - DNA, 6 bp, Artificial Sequence
  synthetic
actatt

SEQ ID NO: 801 - DNA, 6 bp, Artificial Sequence
  synthetic
actcat

SEQ ID NO: 802 - DNA, 6 bp, Artificial Sequence
  synthetic
actccg

SEQ ID NO: 803 - DNA, 6 bp, Artificial Sequence
  synthetic
actcga

SEQ ID NO: 804 - DNA, 6 bp, Artificial Sequence
  synthetic
actcgt

SEQ ID NO: 805 - DNA, 6 bp, Artificial Sequence
  synthetic
actgta

SEQ ID NO: 806 - DNA, 6 bp, Artificial Sequence
  synthetic
acttac

SEQ ID NO: 807 - DNA, 6 bp, Artificial Sequence
  synthetic
acttat

SEQ ID NO: 808 - DNA, 6 bp, Artificial Sequence
  synthetic
acttgc

SEQ ID NO: 809 - DNA, 6 bp, Artificial Sequence
  synthetic
agatcg

SEQ ID NO: 810 - DNA, 6 bp, Artificial Sequence
  synthetic
agcatg

SEQ ID NO: 811 - DNA, 6 bp, Artificial Sequence
  synthetic
aggcaa

SEQ ID NO: 812 - DNA, 6 bp, Artificial Sequence
  synthetic
aggggt

SEQ ID NO: 813 - DNA, 6 bp, Artificial Sequence
  synthetic
aggtac

SEQ ID NO: 814 - DNA, 6 bp, Artificial Sequence
  synthetic
aggtat

SEQ ID NO: 815 - DNA, 6 bp, Artificial Sequence
  synthetic
aggttg

SEQ ID NO: 816 - DNA, 6 bp, Artificial Sequence
  synthetic
agtact

SEQ ID NO: 817 - DNA, 6 bp, Artificial Sequence
  synthetic
agtagt

SEQ ID NO: 818 - DNA, 6 bp, Artificial Sequence
  synthetic
agtcac

SEQ ID NO: 819 - DNA, 6 bp, Artificial Sequence
  synthetic
agtctc

SEQ ID NO: 820 - DNA, 6 bp, Artificial Sequence
  synthetic
agttag

SEQ ID NO: 821 - DNA, 6 bp, Artificial Sequence
  synthetic
agttcg

SEQ ID NO: 822 - DNA, 6 bp, Artificial Sequence
  synthetic
ataatt

SEQ ID NO: 823 - DNA, 6 bp, Artificial Sequence
  synthetic
atacac

SEQ ID NO: 824 - DNA, 6 bp, Artificial Sequence
  synthetic
atacga

SEQ ID NO: 825 - DNA, 6 bp, Artificial Sequence
  synthetic
atacgc

SEQ ID NO: 826 - DNA, 6 bp, Artificial Sequence
  synthetic
atacgt

SEQ ID NO: 827 - DNA, 6 bp, Artificial Sequence
  synthetic
atactg

SEQ ID NO: 828 - DNA, 6 bp, Artificial Sequence
  synthetic
atagtt

SEQ ID NO: 829 - DNA, 6 bp, Artificial Sequence
  synthetic
atatat

SEQ ID NO: 830 - DNA, 6 bp, Artificial Sequence
  synthetic
atatcg

SEQ ID NO: 831 - DNA, 6 bp, Artificial Sequence
  synthetic
atatga

SEQ ID NO: 832 - DNA, 6 bp, Artificial Sequence
  synthetic
atatgg

SEQ ID NO: 833 - DNA, 6 bp, Artificial Sequence
  synthetic
atatgt

SEQ ID NO: 834 - DNA, 6 bp, Artificial Sequence
  synthetic
atattg

SEQ ID NO: 835 - DNA, 6 bp, Artificial Sequence
  synthetic
atattt

SEQ ID NO: 836 - DNA, 6 bp, Artificial Sequence
  synthetic
atcacg

SEQ ID NO: 837 - DNA, 6 bp, Artificial Sequence
  synthetic
atcata

SEQ ID NO: 838 - DNA, 6 bp, Artificial Sequence
  synthetic
atccac

SEQ ID NO: 839 - DNA, 6 bp, Artificial Sequence
  synthetic
atcctc

SEQ ID NO: 840 - DNA, 6 bp, Artificial Sequence
  synthetic
atcgac

SEQ ID NO: 841 - DNA, 6 bp, Artificial Sequence
  synthetic
atcgct

SEQ ID NO: 842 - DNA, 6 bp, Artificial Sequence
  synthetic
atcgga

SEQ ID NO: 843 - DNA, 6 bp, Artificial Sequence
  synthetic
atcgta

SEQ ID NO: 844 - DNA, 6 bp, Artificial Sequence
  synthetic
atcgtc

SEQ ID NO: 845 - DNA, 6 bp, Artificial Sequence
  synthetic
atcgtg

SEQ ID NO: 846 - DNA, 6 bp, Artificial Sequence
  synthetic
atctcc

SEQ ID NO: 847 - DNA, 6 bp, Artificial Sequence
  synthetic
atctct

SEQ ID NO: 848 - DNA, 6 bp, Artificial Sequence
  synthetic
atctgc

SEQ ID NO: 849 - DNA, 6 bp, Artificial Sequence
  synthetic
atgaca

SEQ ID NO: 850 - DNA, 6 bp, Artificial Sequence
  synthetic
atgacg

SEQ ID NO: 851 - DNA, 6 bp, Artificial Sequence
  synthetic
atgagc

SEQ ID NO: 852 - DNA, 6 bp, Artificial Sequence
  synthetic
atgcac

SEQ ID NO: 853 - DNA, 6 bp, Artificial Sequence
  synthetic
atgcgc

SEQ ID NO: 854 - DNA, 6 bp, Artificial Sequence
  synthetic
atggga

SEQ ID NO: 855 - DNA, 6 bp, Artificial Sequence
  synthetic
atggtc

SEQ ID NO: 856 - DNA, 6 bp, Artificial Sequence
  synthetic
atgtaa

SEQ ID NO: 857 - DNA, 6 bp, Artificial Sequence
  synthetic
atgtac

SEQ ID NO: 858 - DNA, 6 bp, Artificial Sequence
  synthetic
atgtat

SEQ ID NO: 859 - DNA, 6 bp, Artificial Sequence
  synthetic
attacg

SEQ ID NO: 860 - DNA, 6 bp, Artificial Sequence
  synthetic
attagg

SEQ ID NO: 861 - DNA, 6 bp, Artificial Sequence
  synthetic
attatc

SEQ ID NO: 862 - DNA, 6 bp, Artificial Sequence
  synthetic
attcat

SEQ ID NO: 863 - DNA, 6 bp, Artificial Sequence
  synthetic
attcgg

SEQ ID NO: 864 - DNA, 6 bp, Artificial Sequence
  synthetic
attgcc

SEQ ID NO: 865 - DNA, 6 bp, Artificial Sequence
  synthetic
attgct

SEQ ID NO: 866 - DNA, 6 bp, Artificial Sequence
  synthetic
attggg

SEQ ID NO: 867 - DNA, 6 bp, Artificial Sequence
  synthetic
attggt

SEQ ID NO: 868 - DNA, 6 bp, Artificial Sequence
  synthetic
attgta

SEQ ID NO: 869 - DNA, 6 bp, Artificial Sequence
  synthetic
attgtc

SEQ ID NO: 870 - DNA, 6 bp, Artificial Sequence
  synthetic
atttta

SEQ ID NO: 871 - DNA, 6 bp, Artificial Sequence
  synthetic
attttt

SEQ ID NO: 872 - DNA, 6 bp, Artificial Sequence
  synthetic
caacat

SEQ ID NO: 873 - DNA, 6 bp, Artificial Sequence
  synthetic
caaccg

SEQ ID NO: 874 - DNA, 6 bp, Artificial Sequence
  synthetic
caatcg

SEQ ID NO: 875 - DNA, 6 bp, Artificial Sequence
  synthetic
caattc

SEQ ID NO: 876 - DNA, 6 bp, Artificial Sequence
  synthetic
cacagt

SEQ ID NO: 877 - DNA, 6 bp, Artificial Sequence
  synthetic
cacata

SEQ ID NO: 878 - DNA, 6 bp, Artificial Sequence
  synthetic
cacatt

SEQ ID NO: 879 - DNA, 6 bp, Artificial Sequence
  synthetic
caccga

SEQ ID NO: 880 - DNA, 6 bp, Artificial Sequence
  synthetic
caccta

SEQ ID NO: 881 - DNA, 6 bp, Artificial Sequence
  synthetic
cacgac

SEQ ID NO: 882 - DNA, 6 bp, Artificial Sequence
  synthetic
cactca

SEQ ID NO: 883 - DNA, 6 bp, Artificial Sequence
  synthetic
cactct

SEQ ID NO: 884 - DNA, 6 bp, Artificial Sequence
  synthetic
cactgg

SEQ ID NO: 885 - DNA, 6 bp, Artificial Sequence
  synthetic
cactta

SEQ ID NO: 886 - DNA, 6 bp, Artificial Sequence
  synthetic
cagagt

SEQ ID NO: 887 - DNA, 6 bp, Artificial Sequence
  synthetic
cagatg

SEQ ID NO: 888 - DNA, 6 bp, Artificial Sequence
  synthetic
cagcat

SEQ ID NO: 889 - DNA, 6 bp, Artificial Sequence
  synthetic
caggaa

SEQ ID NO: 890 - DNA, 6 bp, Artificial Sequence
  synthetic
caggta

SEQ ID NO: 891 - DNA, 6 bp, Artificial Sequence
  synthetic
cagtct

SEQ ID NO: 892 - DNA, 6 bp, Artificial Sequence
  synthetic
cagttc

SEQ ID NO: 893 - DNA, 6 bp, Artificial Sequence
  synthetic
cataca

SEQ ID NO: 894 - DNA, 6 bp, Artificial Sequence
  synthetic
catacg

SEQ ID NO: 895 - DNA, 6 bp, Artificial Sequence
  synthetic
catact

SEQ ID NO: 896 - DNA, 6 bp, Artificial Sequence
  synthetic
cataga

SEQ ID NO: 897 - DNA, 6 bp, Artificial Sequence
  synthetic
catagg

SEQ ID NO: 898 - DNA, 6 bp, Artificial Sequence
  synthetic
catccg

SEQ ID NO: 899 - DNA, 6 bp, Artificial Sequence
  synthetic
catcct

SEQ ID NO: 900 - DNA, 6 bp, Artificial Sequence
  synthetic
catcga

SEQ ID NO: 901 - DNA, 6 bp, Artificial Sequence
  synthetic
catcgg

SEQ ID NO: 902 - DNA, 6 bp, Artificial Sequence
  synthetic
catcgt

SEQ ID NO: 903 - DNA, 6 bp, Artificial Sequence
  synthetic
catgcg

SEQ ID NO: 904 - DNA, 6 bp, Artificial Sequence
  synthetic
catgta

SEQ ID NO: 905 - DNA, 6 bp, Artificial Sequence
  synthetic
cattac

SEQ ID NO: 906 - DNA, 6 bp, Artificial Sequence
  synthetic
cattag

SEQ ID NO: 907 - DNA, 6 bp, Artificial Sequence
  synthetic
cattca

SEQ ID NO: 908 - DNA, 6 bp, Artificial Sequence
  synthetic
cattgc

SEQ ID NO: 909 - DNA, 6 bp, Artificial Sequence
  synthetic
ccatcc

SEQ ID NO: 910 - DNA, 6 bp, Artificial Sequence
  synthetic
ccatcg

SEQ ID NO: 911 - DNA, 6 bp, Artificial Sequence
  synthetic
ccatta

SEQ ID NO: 912 - DNA, 6 bp, Artificial Sequence
  synthetic
ccgctt

SEQ ID NO: 913 - DNA, 6 bp, Artificial Sequence
  synthetic
ccggtt

SEQ ID NO: 914 - DNA, 6 bp, Artificial Sequence
  synthetic
ccgtat

SEQ ID NO: 915 - DNA, 6 bp, Artificial Sequence
  synthetic
cctaca

SEQ ID NO: 916 - DNA, 6 bp, Artificial Sequence
  synthetic
ccttca

SEQ ID NO: 917 - DNA, 6 bp, Artificial Sequence
  synthetic
cgaaat

SEQ ID NO: 918 - DNA, 6 bp, Artificial Sequence
  synthetic
cgaaca

SEQ ID NO: 919 - DNA, 6 bp, Artificial Sequence
  synthetic
cgaatt

SEQ ID NO: 920 - DNA, 6 bp, Artificial Sequence
  synthetic
cgacag

SEQ ID NO: 921 - DNA, 6 bp, Artificial Sequence
  synthetic
cgacta

SEQ ID NO: 922 - DNA, 6 bp, Artificial Sequence
  synthetic
cgactc

SEQ ID NO: 923 - DNA, 6 bp, Artificial Sequence
  synthetic
cgatat

SEQ ID NO: 924 - DNA, 6 bp, Artificial Sequence
  synthetic
cgatga

SEQ ID NO: 925 - DNA, 6 bp, Artificial Sequence
  synthetic
cgcatc

SEQ ID NO: 926 - DNA, 6 bp, Artificial Sequence
  synthetic
cgcgtt

SEQ ID NO: 927 - DNA, 6 bp, Artificial Sequence
  synthetic
cggatg

SEQ ID NO: 928 - DNA, 6 bp, Artificial Sequence
  synthetic
cggatt

SEQ ID NO: 929 - DNA, 6 bp, Artificial Sequence
  synthetic
cggcat

SEQ ID NO: 930 - DNA, 6 bp, Artificial Sequence
  synthetic
cggcct

SEQ ID NO: 931 - DNA, 6 bp, Artificial Sequence
  synthetic
cggtat

SEQ ID NO: 932 - DNA, 6 bp, Artificial Sequence
  synthetic
cggtct

SEQ ID NO: 933 - DNA, 6 bp, Artificial Sequence
  synthetic
cggtta

SEQ ID NO: 934 - DNA, 6 bp, Artificial Sequence
  synthetic
cgtaat

SEQ ID NO: 935 - DNA, 6 bp, Artificial Sequence
  synthetic
cgtact

SEQ ID NO: 936 - DNA, 6 bp, Artificial Sequence
  synthetic
cgtatc

SEQ ID NO: 937 - DNA, 6 bp, Artificial Sequence
  synthetic
cgtatg

SEQ ID NO: 938 - DNA, 6 bp, Artificial Sequence
  synthetic
cgtcga

SEQ ID NO: 939 - DNA, 6 bp, Artificial Sequence
  synthetic
cgtgac

SEQ ID NO: 940 - DNA, 6 bp, Artificial Sequence
  synthetic
cgtgta

SEQ ID NO: 941 - DNA, 6 bp, Artificial Sequence
  synthetic
cgttgt

SEQ ID NO: 942 - DNA, 6 bp, Artificial Sequence
  synthetic
cgtttc

SEQ ID NO: 943 - DNA, 6 bp, Artificial Sequence
  synthetic
ctaaag

SEQ ID NO: 944 - DNA, 6 bp, Artificial Sequence
  synthetic
ctaacg

SEQ ID NO: 945 - DNA, 6 bp, Artificial Sequence
  synthetic
ctacag

SEQ ID NO: 946 - DNA, 6 bp, Artificial Sequence
  synthetic
ctacgg

SEQ ID NO: 947 - DNA, 6 bp, Artificial Sequence
  synthetic
ctagac

SEQ ID NO: 948 - DNA, 6 bp, Artificial Sequence
  synthetic
ctagcg

SEQ ID NO: 949 - DNA, 6 bp, Artificial Sequence
  synthetic
ctagct

SEQ ID NO: 950 - DNA, 6 bp, Artificial Sequence
  synthetic
ctaggc

SEQ ID NO: 951 - DNA, 6 bp, Artificial Sequence
  synthetic
ctataa

SEQ ID NO: 952 - DNA, 6 bp, Artificial Sequence
  synthetic
ctatcg

SEQ ID NO: 953 - DNA, 6 bp, Artificial Sequence
  synthetic
ctcgaa

SEQ ID NO: 954 - DNA, 6 bp, Artificial Sequence
  synthetic
ctcgag

SEQ ID NO: 955 - DNA, 6 bp, Artificial Sequence
  synthetic
ctcgtt

SEQ ID NO: 956 - DNA, 6 bp, Artificial Sequence
  synthetic
ctctac

SEQ ID NO: 957 - DNA, 6 bp, Artificial Sequence
  synthetic
ctctat

SEQ ID NO: 958 - DNA, 6 bp, Artificial Sequence
  synthetic
ctctca

SEQ ID NO: 959 - DNA, 6 bp, Artificial Sequence
  synthetic
ctctgt

SEQ ID NO: 960 - DNA, 6 bp, Artificial Sequence
  synthetic
ctgatt

SEQ ID NO: 961 - DNA, 6 bp, Artificial Sequence
  synthetic
ctgcgc

SEQ ID NO: 962 - DNA, 6 bp, Artificial Sequence
  synthetic
ctggta

SEQ ID NO: 963 - DNA, 6 bp, Artificial Sequence
  synthetic
ctgtag

SEQ ID NO: 964 - DNA, 6 bp, Artificial Sequence
  synthetic
ctgtcg

SEQ ID NO: 965 - DNA, 6 bp, Artificial Sequence
  synthetic
ctgtgc

SEQ ID NO: 966 - DNA, 6 bp, Artificial Sequence
  synthetic
cttaac

SEQ ID NO: 967 - DNA, 6 bp, Artificial Sequence
  synthetic
cttaca

SEQ ID NO: 968 - DNA, 6 bp, Artificial Sequence
  synthetic
cttacg

SEQ ID NO: 969 - DNA, 6 bp, Artificial Sequence
  synthetic
cttagg

SEQ ID NO: 970 - DNA, 6 bp, Artificial Sequence
  synthetic
cttata

SEQ ID NO: 971 - DNA, 6 bp, Artificial Sequence
  synthetic
cttatc

SEQ ID NO: 972 - DNA, 6 bp, Artificial Sequence
  synthetic
cttatt

SEQ ID NO: 973 - DNA, 6 bp, Artificial Sequence
  synthetic
cttcca

SEQ ID NO: 974 - DNA, 6 bp, Artificial Sequence
  synthetic
cttcta

SEQ ID NO: 975 - DNA, 6 bp, Artificial Sequence
  synthetic
cttctc

SEQ ID NO: 976 - DNA, 6 bp, Artificial Sequence
  synthetic
ctttac

SEQ ID NO: 977 - DNA, 6 bp, Artificial Sequence
  synthetic
gaaatc

SEQ ID NO: 978 - DNA, 6 bp, Artificial Sequence
  synthetic
gaatat

SEQ ID NO: 979 - DNA, 6 bp, Artificial Sequence
  synthetic
gaatcg

SEQ ID NO: 980 - DNA, 6 bp, Artificial Sequence
  synthetic
gacgta

SEQ ID NO: 981 - DNA, 6 bp, Artificial Sequence
  synthetic
gactag

SEQ ID NO: 982 - DNA, 6 bp, Artificial Sequence
  synthetic
gactcg

SEQ ID NO: 983 - DNA, 6 bp, Artificial Sequence
  synthetic
gacttg

SEQ ID NO: 984 - DNA, 6 bp, Artificial Sequence
  synthetic
gacttt

SEQ ID NO: 985 - DNA, 6 bp, Artificial Sequence
  synthetic
gagaat

SEQ ID NO: 986 - DNA, 6 bp, Artificial Sequence
  synthetic
gagacg

SEQ ID NO: 987 - DNA, 6 bp, Artificial Sequence
  synthetic
gagata

SEQ ID NO: 988 - DNA, 6 bp, Artificial Sequence
  synthetic
gaggct

SEQ ID NO: 989 - DNA, 6 bp, Artificial Sequence
  synthetic
gagtac

SEQ ID NO: 990 - DNA, 6 bp, Artificial Sequence
  synthetic
gagtca

SEQ ID NO: 991 - DNA, 6 bp, Artificial Sequence
  synthetic
gagtta

SEQ ID NO: 992 - DNA, 6 bp, Artificial Sequence
  synthetic
gataat

SEQ ID NO: 993 - DNA, 6 bp, Artificial Sequence
  synthetic
gataca

SEQ ID NO: 994 - DNA, 6 bp, Artificial Sequence
  synthetic
gatact

SEQ ID NO: 995 - DNA, 6 bp, Artificial Sequence
  synthetic
gatatg

SEQ ID NO: 996 - DNA, 6 bp, Artificial Sequence
  synthetic
gatgac

SEQ ID NO: 997 - DNA, 6 bp, Artificial Sequence
  synthetic
gatgag

SEQ ID NO: 998 - DNA, 6 bp, Artificial Sequence
  synthetic
gatgga

SEQ ID NO: 999 - DNA, 6 bp, Artificial Sequence
  synthetic
gatgta

SEQ ID NO: 1000 - DNA, 6 bp, Artificial Sequence
  synthetic
gattcg

SEQ ID NO: 1001 - DNA, 6 bp, Artificial Sequence
  synthetic
gcaaca

SEQ ID NO: 1002 - DNA, 6 bp, Artificial Sequence
  synthetic
gcacag

SEQ ID NO: 1003 - DNA, 6 bp, Artificial Sequence
  synthetic
gcacta

SEQ ID NO: 1004 - DNA, 6 bp, Artificial Sequence
  synthetic
gcatac

SEQ ID NO: 1005 - DNA, 6 bp, Artificial Sequence
  synthetic
gcatag

SEQ ID NO: 1006 - DNA, 6 bp, Artificial Sequence
  synthetic
gcattg

SEQ ID NO: 1007 - DNA, 6 bp, Artificial Sequence
  synthetic
gccaac

SEQ ID NO: 1008 - DNA, 6 bp, Artificial Sequence
  synthetic
gccatt

SEQ ID NO: 1009 - DNA, 6 bp, Artificial Sequence
  synthetic
gcctta

SEQ ID NO: 1010 - DNA, 6 bp, Artificial Sequence
  synthetic
gcgact

SEQ ID NO: 1011 - DNA, 6 bp, Artificial Sequence
  synthetic
gcgctt

SEQ ID NO: 1012 - DNA, 6 bp, Artificial Sequence
  synthetic
gcgtag

SEQ ID NO: 1013 - DNA, 6 bp, Artificial Sequence
  synthetic
gctagc

SEQ ID NO: 1014 - DNA, 6 bp, Artificial Sequence
  synthetic
gctagt

SEQ ID NO: 1015 - DNA, 6 bp, Artificial Sequence
  synthetic
gctatc

SEQ ID NO: 1016 - DNA, 6 bp, Artificial Sequence
  synthetic
gctatg

SEQ ID NO: 1017 - DNA, 6 bp, Artificial Sequence
  synthetic
gctcga

SEQ ID NO: 1018 - DNA, 6 bp, Artificial Sequence
  synthetic
gctgat

SEQ ID NO: 1019 - DNA, 6 bp, Artificial Sequence
  synthetic
gctgta

SEQ ID NO: 1020 - DNA, 6 bp, Artificial Sequence
  synthetic
gctgtg

SEQ ID NO: 1021 - DNA, 6 bp, Artificial Sequence
  synthetic
gcttac

SEQ ID NO: 1022 - DNA, 6 bp, Artificial Sequence
  synthetic
gcttat

SEQ ID NO: 1023 - DNA, 6 bp, Artificial Sequence
  synthetic
ggaagc

SEQ ID NO: 1024 - DNA, 6 bp, Artificial Sequence
  synthetic
ggacgt

SEQ ID NO: 1025 - DNA, 6 bp, Artificial Sequence
  synthetic
ggactt

SEQ ID NO: 1026 - DNA, 6 bp, Artificial Sequence
  synthetic
ggcatc

SEQ ID NO: 1027 - DNA, 6 bp, Artificial Sequence
  synthetic
ggctag

SEQ ID NO: 1028 - DNA, 6 bp, Artificial Sequence
  synthetic
ggctat

SEQ ID NO: 1029 - DNA, 6 bp, Artificial Sequence
  synthetic
ggctgt

SEQ ID NO: 1030 - DNA, 6 bp, Artificial Sequence
  synthetic
gggact

SEQ ID NO: 1031 - DNA, 6 bp, Artificial Sequence
  synthetic
gggtac

SEQ ID NO: 1032 - DNA, 6 bp, Artificial Sequence
  synthetic
gggtag

SEQ ID NO: 1033 - DNA, 6 bp, Artificial Sequence
  synthetic
ggtacg

SEQ ID NO: 1034 - DNA, 6 bp, Artificial Sequence
  synthetic
ggtact

SEQ ID NO: 1035 - DNA, 6 bp, Artificial Sequence
  synthetic
ggtagg

SEQ ID NO: 1036 - DNA, 6 bp, Artificial Sequence
  synthetic
ggtatc

SEQ ID NO: 1037 - DNA, 6 bp, Artificial Sequence
  synthetic
ggtatt

SEQ ID NO: 1038 - DNA, 6 bp, Artificial Sequence
  synthetic
ggtcca

SEQ ID NO: 1039 - DNA, 6 bp, Artificial Sequence
  synthetic
ggttac

SEQ ID NO: 1040 - DNA, 6 bp, Artificial Sequence
  synthetic
gtaata

SEQ ID NO: 1041 - DNA, 6 bp, Artificial Sequence
  synthetic
gtaatg

SEQ ID NO: 1042 - DNA, 6 bp, Artificial Sequence
  synthetic
gtacaa

SEQ ID NO: 1043 - DNA, 6 bp, Artificial Sequence
  synthetic
gtacta

SEQ ID NO: 1044 - DNA, 6 bp, Artificial Sequence
  synthetic
gtactc

SEQ ID NO: 1045 - DNA, 6 bp, Artificial Sequence
  synthetic
gtactt

SEQ ID NO: 1046 - DNA, 6 bp, Artificial Sequence
  synthetic
gtagat

SEQ ID NO: 1047 - DNA, 6 bp, Artificial Sequence
  synthetic
gtaggg

SEQ ID NO: 1048 - DNA, 6 bp, Artificial Sequence
  synthetic
gtatcc

SEQ ID NO: 1049 - DNA, 6 bp, Artificial Sequence
  synthetic
gtatcg

SEQ ID NO: 1050 - DNA, 6 bp, Artificial Sequence
  synthetic
gtatct

SEQ ID NO: 1051 - DNA, 6 bp, Artificial Sequence
  synthetic
gtatgc

SEQ ID NO: 1052 - DNA, 6 bp, Artificial Sequence
  synthetic
gtattc

SEQ ID NO: 1053 - DNA, 6 bp, Artificial Sequence
  synthetic
gtattt

SEQ ID NO: 1054 - DNA, 6 bp, Artificial Sequence
  synthetic
gtcact

SEQ ID NO: 1055 - DNA, 6 bp, Artificial Sequence
  synthetic
gtcagg

SEQ ID NO: 1056 - DNA, 6 bp, Artificial Sequence
  synthetic
gtcatg

SEQ ID NO: 1057 - DNA, 6 bp, Artificial Sequence
  synthetic
gtccca

SEQ ID NO: 1058 - DNA, 6 bp, Artificial Sequence
  synthetic
gtcgac

SEQ ID NO: 1059 - DNA, 6 bp, Artificial Sequence
  synthetic
gtcgat

SEQ ID NO: 1060 - DNA, 6 bp, Artificial Sequence
  synthetic
gtcgca

SEQ ID NO: 1061 - DNA, 6 bp, Artificial Sequence
  synthetic
gtcgtt

SEQ ID NO: 1062 - DNA, 6 bp, Artificial Sequence
  synthetic
gtctag

SEQ ID NO: 1063 - DNA, 6 bp, Artificial Sequence
  synthetic
gtctta

SEQ ID NO: 1064 - DNA, 6 bp, Artificial Sequence
  synthetic
gtgcga

SEQ ID NO: 1065 - DNA, 6 bp, Artificial Sequence
  synthetic
gtggtt

SEQ ID NO: 1066 - DNA, 6 bp, Artificial Sequence
  synthetic
gtgtct

SEQ ID NO: 1067 - DNA, 6 bp, Artificial Sequence
  synthetic
gttaac

SEQ ID NO: 1068 - DNA, 6 bp, Artificial Sequence
  synthetic
gttaga

SEQ ID NO: 1069 - DNA, 6 bp, Artificial Sequence
  synthetic
gttagc

SEQ ID NO: 1070 - DNA, 6 bp, Artificial Sequence
  synthetic
gttata

SEQ ID NO: 1071 - DNA, 6 bp, Artificial Sequence
  synthetic
gttcgg

SEQ ID NO: 1072 - DNA, 6 bp, Artificial Sequence
  synthetic
gttgcg

SEQ ID NO: 1073 - DNA, 6 bp, Artificial Sequence
  synthetic
gttgtg

SEQ ID NO: 1074 - DNA, 6 bp, Artificial Sequence
  synthetic
gtttat

SEQ ID NO: 1075 - DNA, 6 bp, Artificial Sequence
  synthetic
gtttca

SEQ ID NO: 1076 - DNA, 6 bp, Artificial Sequence
  synthetic
gtttgc

SEQ ID NO: 1077 - DNA, 6 bp, Artificial Sequence
  synthetic
taaaat

SEQ ID NO: 1078 - DNA, 6 bp, Artificial Sequence
  synthetic
taaaca

SEQ ID NO: 1079 - DNA, 6 bp, Artificial Sequence
  synthetic
taacgt

SEQ ID NO: 1080 - DNA, 6 bp, Artificial Sequence
  synthetic
taactc

SEQ ID NO: 1081 - DNA, 6 bp, Artificial Sequence
  synthetic
taagtt

SEQ ID NO: 1082 - DNA, 6 bp, Artificial Sequence
  synthetic
taatct

SEQ ID NO: 1083 - DNA, 6 bp, Artificial Sequence
  synthetic
tacaac

SEQ ID NO: 1084 - DNA, 6 bp, Artificial Sequence
  synthetic
tacaag

SEQ ID NO: 1085 - DNA, 6 bp, Artificial Sequence
  synthetic
tacacg

SEQ ID NO: 1086 - DNA, 6 bp, Artificial Sequence
  synthetic
tacata

SEQ ID NO: 1087 - DNA, 6 bp, Artificial Sequence
  synthetic
tacatc

SEQ ID NO: 1088 - DNA, 6 bp, Artificial Sequence
  synthetic
tacctc

SEQ ID NO: 1089 - DNA, 6 bp, Artificial Sequence
  synthetic
tacgct

SEQ ID NO: 1090 - DNA, 6 bp, Artificial Sequence
  synthetic
tacggg

SEQ ID NO: 1091 - DNA, 6 bp, Artificial Sequence
  synthetic
tacggt

SEQ ID NO: 1092 - DNA, 6 bp, Artificial Sequence
  synthetic
tacgtc

SEQ ID NO: 1093 - DNA, 6 bp, Artificial Sequence
  synthetic
tacgtt

SEQ ID NO: 1094 - DNA, 6 bp, Artificial Sequence
  synthetic
tactag

SEQ ID NO: 1095 - DNA, 6 bp, Artificial Sequence
  synthetic
tactcc

SEQ ID NO: 1096 - DNA, 6 bp, Artificial Sequence
  synthetic
tactcg

SEQ ID NO: 1097 - DNA, 6 bp, Artificial Sequence
  synthetic
tactgt

SEQ ID NO: 1098 - DNA, 6 bp, Artificial Sequence
  synthetic
tactta

SEQ ID NO: 1099 - DNA, 6 bp, Artificial Sequence
  synthetic
tagcac

SEQ ID NO: 1100 - DNA, 6 bp, Artificial Sequence
  synthetic
tagcgc

SEQ ID NO: 1101 - DNA, 6 bp, Artificial Sequence
  synthetic
tagctt

SEQ ID NO: 1102 - DNA, 6 bp, Artificial Sequence
  synthetic
taggat

SEQ ID NO: 1103 - DNA, 6 bp, Artificial Sequence
  synthetic
taggca

SEQ ID NO: 1104 - DNA, 6 bp, Artificial Sequence
  synthetic
tagtgc

SEQ ID NO: 1105 - DNA, 6 bp, Artificial Sequence
  synthetic
tagtgt

SEQ ID NO: 1106 - DNA, 6 bp, Artificial Sequence
  synthetic
tataaa

SEQ ID NO: 1107 - DNA, 6 bp, Artificial Sequence
  synthetic
tataat

SEQ ID NO: 1108 - DNA, 6 bp, Artificial Sequence
  synthetic
tataca

SEQ ID NO: 1109 - DNA, 6 bp, Artificial Sequence
  synthetic
tatacg

SEQ ID NO: 1110 - DNA, 6 bp, Artificial Sequence
  synthetic
tatatc

SEQ ID NO: 1111 - DNA, 6 bp, Artificial Sequence
  synthetic
tatatg

SEQ ID NO: 1112 - DNA, 6 bp, Artificial Sequence
  synthetic
tatcct

SEQ ID NO: 1113 - DNA, 6 bp, Artificial Sequence
  synthetic
tatcga

SEQ ID NO: 1114 - DNA, 6 bp, Artificial Sequence
  synthetic
tatcgc

SEQ ID NO: 1115 - DNA, 6 bp, Artificial Sequence
  synthetic
tatcgg

SEQ ID NO: 1116 - DNA, 6 bp, Artificial Sequence
  synthetic
tatcgt

SEQ ID NO: 1117 - DNA, 6 bp, Artificial Sequence
  synthetic
tatctc

SEQ ID NO: 1118 - DNA, 6 bp, Artificial Sequence
  synthetic
tatctt

SEQ ID NO: 1119 - DNA, 6 bp, Artificial Sequence
  synthetic
tatgag

SEQ ID NO: 1120 - DNA, 6 bp, Artificial Sequence
  synthetic
tatgat

SEQ ID NO: 1121 - DNA, 6 bp, Artificial Sequence
  synthetic
tatgca

SEQ ID NO: 1122 - DNA, 6 bp, Artificial Sequence
  synthetic
tatgcg

SEQ ID NO: 1123 - DNA, 6 bp, Artificial Sequence
  synthetic
tatgtc

SEQ ID NO: 1124 - DNA, 6 bp, Artificial Sequence
  synthetic
tatgtt

SEQ ID NO: 1125 - DNA, 6 bp, Artificial Sequence
  synthetic
tattcg

SEQ ID NO: 1126 - DNA, 6 bp, Artificial Sequence
  synthetic
tattgg

SEQ ID NO: 1127 - DNA, 6 bp, Artificial Sequence
  synthetic
tattgt

SEQ ID NO: 1128 - DNA, 6 bp, Artificial Sequence
  synthetic
tattta

SEQ ID NO: 1129 - DNA, 6 bp, Artificial Sequence
  synthetic
tatttg

SEQ ID NO: 1130 - DNA, 6 bp, Artificial Sequence
  synthetic
tcaatc

SEQ ID NO: 1131 - DNA, 6 bp, Artificial Sequence
  synthetic
tcacat

SEQ ID NO: 1132 - DNA, 6 bp, Artificial Sequence
  synthetic
tcaccg

SEQ ID NO: 1133 - DNA, 6 bp, Artificial Sequence
  synthetic
tcacgg

SEQ ID NO: 1134 - DNA, 6 bp, Artificial Sequence
  synthetic
tcacgt

SEQ ID NO: 1135 - DNA, 6 bp, Artificial Sequence
  synthetic
tcactc

SEQ ID NO: 1136 - DNA, 6 bp, Artificial Sequence
  synthetic
tcaggt

SEQ ID NO: 1137 - DNA, 6 bp, Artificial Sequence
  synthetic
tcagtg

SEQ ID NO: 1138 - DNA, 6 bp, Artificial Sequence
  synthetic
tcatcc

SEQ ID NO: 1139 - DNA, 6 bp, Artificial Sequence
  synthetic
tcatcg

SEQ ID NO: 1140 - DNA, 6 bp, Artificial Sequence
  synthetic
tcatga

SEQ ID NO: 1141 - DNA, 6 bp, Artificial Sequence
  synthetic
tcatgc

SEQ ID NO: 1142 - DNA, 6 bp, Artificial Sequence
  synthetic
tcatgt

SEQ ID NO: 1143 - DNA, 6 bp, Artificial Sequence
  synthetic
tcattc

SEQ ID NO: 1144 - DNA, 6 bp, Artificial Sequence
  synthetic
tccaca

SEQ ID NO: 1145 - DNA, 6 bp, Artificial Sequence
  synthetic
tcccag

SEQ ID NO: 1146 - DNA, 6 bp, Artificial Sequence
  synthetic
tcgaat

SEQ ID NO: 1147 - DNA, 6 bp, Artificial Sequence
  synthetic
tcgacg

SEQ ID NO: 1148 - DNA, 6 bp, Artificial Sequence
  synthetic
tcgact

SEQ ID NO: 1149 - DNA, 6 bp, Artificial Sequence
  synthetic
tcgagc

SEQ ID NO: 1150 - DNA, 6 bp, Artificial Sequence
  synthetic
tcgagt

SEQ ID NO: 1151 - DNA, 6 bp, Artificial Sequence
  synthetic
tcgatc

SEQ ID NO: 1152 - DNA, 6 bp, Artificial Sequence
  synthetic
tcgcaa

SEQ ID NO: 1153 - DNA, 6 bp, Artificial Sequence
  synthetic
tcgcat

SEQ ID NO: 1154 - DNA, 6 bp, Artificial Sequence
  synthetic
tcgcgt

SEQ ID NO: 1155 - DNA, 6 bp, Artificial Sequence
  synthetic
tcggac

SEQ ID NO: 1156 - DNA, 6 bp, Artificial Sequence
  synthetic
tcgtcg

SEQ ID NO: 1157 - DNA, 6 bp, Artificial Sequence
  synthetic
tcgtct

SEQ ID NO: 1158 - DNA, 6 bp, Artificial Sequence
  synthetic
tcgtgt

SEQ ID NO: 1159 - DNA, 6 bp, Artificial Sequence
  synthetic
tcgtta

SEQ ID NO: 1160 - DNA, 6 bp, Artificial Sequence
  synthetic
tcgttc

SEQ ID NO: 1161 - DNA, 6 bp, Artificial Sequence
  synthetic
tcgttg

SEQ ID NO: 1162 - DNA, 6 bp, Artificial Sequence
  synthetic
tctacg

SEQ ID NO: 1163 - DNA, 6 bp, Artificial Sequence
  synthetic
tctagg

SEQ ID NO: 1164 - DNA, 6 bp, Artificial Sequence
  synthetic
tctata

SEQ ID NO: 1165 - DNA, 6 bp, Artificial Sequence
  synthetic
tctcac

SEQ ID NO: 1166 - DNA, 6 bp, Artificial Sequence
  synthetic
tctcat

SEQ ID NO: 1167 - DNA, 6 bp, Artificial Sequence
  synthetic
tctcgt

SEQ ID NO: 1168 - DNA, 6 bp, Artificial Sequence
  synthetic
tctcta

SEQ ID NO: 1169 - DNA, 6 bp, Artificial Sequence
  synthetic
tctctg

SEQ ID NO: 1170 - DNA, 6 bp, Artificial Sequence
  synthetic
tctgcg

SEQ ID NO: 1171 - DNA, 6 bp, Artificial Sequence
  synthetic
tctgtt

SEQ ID NO: 1172 - DNA, 6 bp, Artificial Sequence
  synthetic
tcttat

SEQ ID NO: 1173 - DNA, 6 bp, Artificial Sequence
  synthetic
tcttcg

SEQ ID NO: 1174 - DNA, 6 bp, Artificial Sequence
  synthetic
tcttgt

SEQ ID NO: 1175 - DNA, 6 bp, Artificial Sequence
  synthetic
tcttta

SEQ ID NO: 1176 - DNA, 6 bp, Artificial Sequence
  synthetic
tgaatc

SEQ ID NO: 1177 - DNA, 6 bp, Artificial Sequence
  synthetic
tgaggg

SEQ ID NO: 1178 - DNA, 6 bp, Artificial Sequence
  synthetic
tgagta

SEQ ID NO: 1179 - DNA, 6 bp, Artificial Sequence
  synthetic
tgatac

SEQ ID NO: 1180 - DNA, 6 bp, Artificial Sequence
  synthetic
tgatca

SEQ ID NO: 1181 - DNA, 6 bp, Artificial Sequence
  synthetic
tgattg

SEQ ID NO: 1182 - DNA, 6 bp, Artificial Sequence
  synthetic
tgcaac

SEQ ID NO: 1183 - DNA, 6 bp, Artificial Sequence
  synthetic
tgcaca

SEQ ID NO: 1184 - DNA, 6 bp, Artificial Sequence
  synthetic
tgccgg

SEQ ID NO: 1185 - DNA, 6 bp, Artificial Sequence
  synthetic
tgcgac

SEQ ID NO: 1186 - DNA, 6 bp, Artificial Sequence
  synthetic
tgcgca

SEQ ID NO: 1187 - DNA, 6 bp, Artificial Sequence
  synthetic
tgcgct

SEQ ID NO: 1188 - DNA, 6 bp, Artificial Sequence
  synthetic
tgcgta

SEQ ID NO: 1189 - DNA, 6 bp, Artificial Sequence
  synthetic
tgctac

SEQ ID NO: 1190 - DNA, 6 bp, Artificial Sequence
  synthetic
tgctat

SEQ ID NO: 1191 - DNA, 6 bp, Artificial Sequence
  synthetic
tgctcc

SEQ ID NO: 1192 - DNA, 6 bp, Artificial Sequence
  synthetic
tgcttt

SEQ ID NO: 1193 - DNA, 6 bp, Artificial Sequence
  synthetic
tgggac

SEQ ID NO: 1194 - DNA, 6 bp, Artificial Sequence
  synthetic
tggtac

SEQ ID NO: 1195 - DNA, 6 bp, Artificial Sequence
  synthetic
tggtat

SEQ ID NO: 1196 - DNA, 6 bp, Artificial Sequence
  synthetic
tgtaag

SEQ ID NO: 1197 - DNA, 6 bp, Artificial Sequence
  synthetic
tgtacc

SEQ ID NO: 1198 - DNA, 6 bp, Artificial Sequence
  synthetic
tgtagt

SEQ ID NO: 1199 - DNA, 6 bp, Artificial Sequence
  synthetic
tgtata

SEQ ID NO: 1200 - DNA, 6 bp, Artificial Sequence
  synthetic
tgtatc

SEQ ID NO: 1201 - DNA, 6 bp, Artificial Sequence
  synthetic
tgtatt

SEQ ID NO: 1202 - DNA, 6 bp, Artificial Sequence
  synthetic
tgtcac

SEQ ID NO: 1203 - DNA, 6 bp, Artificial Sequence
  synthetic
tgtcat

SEQ ID NO: 1204 - DNA, 6 bp, Artificial Sequence
  synthetic
tgtcga

SEQ ID NO: 1205 - DNA, 6 bp, Artificial Sequence
  synthetic
tgtcgc

SEQ ID NO: 1206 - DNA, 6 bp, Artificial Sequence
  synthetic
tgtcgt

SEQ ID NO: 1207 - DNA, 6 bp, Artificial Sequence
  synthetic
tgtctt

SEQ ID NO: 1208 - DNA, 6 bp, Artificial Sequence
  synthetic
tgtgca

SEQ ID NO: 1209 - DNA, 6 bp, Artificial Sequence
  synthetic
tgtgtc

SEQ ID NO: 1210 - DNA, 6 bp, Artificial Sequence
  synthetic
tgttaa

SEQ ID NO: 1211 - DNA, 6 bp, Artificial Sequence
  synthetic
tgttcg

SEQ ID NO: 1212 - DNA, 6 bp, Artificial Sequence
  synthetic
tgtttg

SEQ ID NO: 1213 - DNA, 6 bp, Artificial Sequence
  synthetic
ttaaac

SEQ ID NO: 1214 - DNA, 6 bp, Artificial Sequence
  synthetic
ttaata

SEQ ID NO: 1215 - DNA, 6 bp, Artificial Sequence
  synthetic
ttacaa

SEQ ID NO: 1216 - DNA, 6 bp, Artificial Sequence
  synthetic
ttacat

SEQ ID NO: 1217 - DNA, 6 bp, Artificial Sequence
  synthetic
ttaccg

SEQ ID NO: 1218 - DNA, 6 bp, Artificial Sequence
  synthetic
ttacct

SEQ ID NO: 1219 - DNA, 6 bp, Artificial Sequence
  synthetic
ttacgg

SEQ ID NO: 1220 - DNA, 6 bp, Artificial Sequence
  synthetic
ttacgt

SEQ ID NO: 1221 - DNA, 6 bp, Artificial Sequence
  synthetic
ttactc

SEQ ID NO: 1222 - DNA, 6 bp, Artificial Sequence
  synthetic
ttagcg

SEQ ID NO: 1223 - DNA, 6 bp, Artificial Sequence
  synthetic
ttaggc

SEQ ID NO: 1224 - DNA, 6 bp, Artificial Sequence
  synthetic
ttaggg

SEQ ID NO: 1225 - DNA, 6 bp, Artificial Sequence
  synthetic
ttatcg

SEQ ID NO: 1226 - DNA, 6 bp, Artificial Sequence
  synthetic
ttatct

SEQ ID NO: 1227 - DNA, 6 bp, Artificial Sequence
  synthetic
ttatgc

SEQ ID NO: 1228 - DNA, 6 bp, Artificial Sequence
  synthetic
ttatgt

SEQ ID NO: 1229 - DNA, 6 bp, Artificial Sequence
  synthetic
ttattg

SEQ ID NO: 1230 - DNA, 6 bp, Artificial Sequence
  synthetic
ttcacg

SEQ ID NO: 1231 - DNA, 6 bp, Artificial Sequence
  synthetic
ttcatc

SEQ ID NO: 1232 - DNA, 6 bp, Artificial Sequence
  synthetic
ttcatg

SEQ ID NO: 1233 - DNA, 6 bp, Artificial Sequence
  synthetic
ttccaa

SEQ ID NO: 1234 - DNA, 6 bp, Artificial Sequence
  synthetic
ttcgca

SEQ ID NO: 1235 - DNA, 6 bp, Artificial Sequence
  synthetic
ttcgct

SEQ ID NO: 1236 - DNA, 6 bp, Artificial Sequence
  synthetic
ttctaa

SEQ ID NO: 1237 - DNA, 6 bp, Artificial Sequence
  synthetic
ttgagg

SEQ ID NO: 1238 - DNA, 6 bp, Artificial Sequence
  synthetic
ttgatg

SEQ ID NO: 1239 - DNA, 6 bp, Artificial Sequence
  synthetic
ttgcag

SEQ ID NO: 1240 - DNA, 6 bp, Artificial Sequence
  synthetic
ttgcat

SEQ ID NO: 1241 - DNA, 6 bp, Artificial Sequence
  synthetic
ttgccg

SEQ ID NO: 1242 - DNA, 6 bp, Artificial Sequence
  synthetic
ttgcga

SEQ ID NO: 1243 - DNA, 6 bp, Artificial Sequence
  synthetic
ttgcgg

SEQ ID NO: 1244 - DNA, 6 bp, Artificial Sequence
  synthetic
ttgcta

SEQ ID NO: 1245 - DNA, 6 bp, Artificial Sequence
  synthetic
ttgtat

SEQ ID NO: 1246 - DNA, 6 bp, Artificial Sequence
  synthetic
ttgtca

SEQ ID NO: 1247 - DNA, 6 bp, Artificial Sequence
  synthetic
ttgtcg

SEQ ID NO: 1248 - DNA, 6 bp, Artificial Sequence
  synthetic
ttgtgc

SEQ ID NO: 1249 - DNA, 6 bp, Artificial Sequence
  synthetic
ttgtgt

SEQ ID NO: 1250 - DNA, 6 bp, Artificial Sequence
  synthetic
ttgtta

SEQ ID NO: 1251 - DNA, 6 bp, Artificial Sequence
  synthetic
ttgttt

SEQ ID NO: 1252 - DNA, 6 bp, Artificial Sequence
  synthetic
tttaca

SEQ ID NO: 1253 - DNA, 6 bp, Artificial Sequence
  synthetic
tttagg

SEQ ID NO: 1254 - DNA, 6 bp, Artificial Sequence
  synthetic
tttatg

SEQ ID NO: 1255 - DNA, 6 bp, Artificial Sequence
  synthetic
tttcgc

SEQ ID NO: 1256 - DNA, 6 bp, Artificial Sequence
  synthetic
tttgcg

SEQ ID NO: 1257 - DNA, 6 bp, Artificial Sequence
  synthetic
ttttcc

SEQ ID NO: 1258 - DNA, 6 bp, Artificial Sequence
  synthetic
ttttgc

SEQ ID NO: 1259 - DNA, 6 bp, Artificial Sequence
  synthetic
ttttta

SEQ ID NO: 1260 - DNA, 6 bp, Artificial Sequence
  synthetic
aaatgt

SEQ ID NO: 1261 - DNA, 6 bp, Artificial Sequence
  synthetic
aacaga

SEQ ID NO: 1262 - DNA, 6 bp, Artificial Sequence
  synthetic
aagcaa

SEQ ID NO: 1263 - DNA, 6 bp, Artificial Sequence
  synthetic
aaggtc

SEQ ID NO: 1264 - DNA, 6 bp, Artificial Sequence
  synthetic
aagttc

SEQ ID NO: 1265 - DNA, 6 bp, Artificial Sequence
  synthetic
aatgtg

SEQ ID NO: 1266 - DNA, 6 bp, Artificial Sequence
  synthetic
acaaat

SEQ ID NO: 1267 - DNA, 6 bp, Artificial Sequence
  synthetic
acacca

SEQ ID NO: 1268 - DNA, 6 bp, Artificial Sequence
  synthetic
acactc

SEQ ID NO: 1269 - DNA, 6 bp, Artificial Sequence
  synthetic
acactt

SEQ ID NO: 1270 - DNA, 6 bp, Artificial Sequence
  synthetic
acagag

SEQ ID NO: 1271 - DNA, 6 bp, Artificial Sequence
  synthetic
acataa

SEQ ID NO: 1272 - DNA, 6 bp, Artificial Sequence
  synthetic
acccag

SEQ ID NO: 1273 - DNA, 6 bp, Artificial Sequence
  synthetic
accctt

SEQ ID NO: 1274 - DNA, 6 bp, Artificial Sequence
  synthetic
acgaca

SEQ ID NO: 1275 - DNA, 6 bp, Artificial Sequence
  synthetic
acgcca

SEQ ID NO: 1276 - DNA, 6 bp, Artificial Sequence
  synthetic
acgctg

SEQ ID NO: 1277 - DNA, 6 bp, Artificial Sequence
  synthetic
acgtca

SEQ ID NO: 1278 - DNA, 6 bp, Artificial Sequence
  synthetic
actcag

SEQ ID NO: 1279 - DNA, 6 bp, Artificial Sequence
  synthetic
actgca

SEQ ID NO: 1280 - DNA, 6 bp, Artificial Sequence
  synthetic
actgcc

SEQ ID NO: 1281 - DNA, 6 bp, Artificial Sequence
  synthetic
acttcc

SEQ ID NO: 1282 - DNA, 6 bp, Artificial Sequence
  synthetic
agaagt

SEQ ID NO: 1283 - DNA, 6 bp, Artificial Sequence
  synthetic
agacac

SEQ ID NO: 1284 - DNA, 6 bp, Artificial Sequence
  synthetic
agacca

SEQ ID NO: 1285 - DNA, 6 bp, Artificial Sequence
  synthetic
agacct

SEQ ID NO: 1286 - DNA, 6 bp, Artificial Sequence
  synthetic
agacgc

SEQ ID NO: 1287 - DNA, 6 bp, Artificial Sequence
  synthetic
agactg

SEQ ID NO: 1288 - DNA, 6 bp, Artificial Sequence
  synthetic
agatgc

SEQ ID NO: 1289 - DNA, 6 bp, Artificial Sequence
  synthetic
agcaac

SEQ ID NO: 1290 - DNA, 6 bp, Artificial Sequence
  synthetic
agcacc

SEQ ID NO: 1291 - DNA, 6 bp, Artificial Sequence
  synthetic
agccgt

SEQ ID NO: 1292 - DNA, 6 bp, Artificial Sequence
  synthetic
aggatg

SEQ ID NO: 1293 - DNA, 6 bp, Artificial Sequence
  synthetic
aggctc

SEQ ID NO: 1294 - DNA, 6 bp, Artificial Sequence
  synthetic
aggctg

SEQ ID NO: 1295 - DNA, 6 bp, Artificial Sequence
  synthetic
aggctt

SEQ ID NO: 1296 - DNA, 6 bp, Artificial Sequence
  synthetic
agggta

SEQ ID NO: 1297 - DNA, 6 bp, Artificial Sequence
  synthetic
agtatc

SEQ ID NO: 1298 - DNA, 6 bp, Artificial Sequence
  synthetic
agtggt

SEQ ID NO: 1299 - DNA, 6 bp, Artificial Sequence
  synthetic
ataaaa

SEQ ID NO: 1300 - DNA, 6 bp, Artificial Sequence
  synthetic
ataaat

SEQ ID NO: 1301 - DNA, 6 bp, Artificial Sequence
  synthetic
ataaga

SEQ ID NO: 1302 - DNA, 6 bp, Artificial Sequence
  synthetic
atacaa

SEQ ID NO: 1303 - DNA, 6 bp, Artificial Sequence
  synthetic
atcaat

SEQ ID NO: 1304 - DNA, 6 bp, Artificial Sequence
  synthetic
atcaca

SEQ ID NO: 1305 - DNA, 6 bp, Artificial Sequence
  synthetic
atcatg

SEQ ID NO: 1306 - DNA, 6 bp, Artificial Sequence
  synthetic
atctgg

SEQ ID NO: 1307 - DNA, 6 bp, Artificial Sequence
  synthetic
atgatc

SEQ ID NO: 1308 - DNA, 6 bp, Artificial Sequence
  synthetic
atgcca

SEQ ID NO: 1309 - DNA, 6 bp, Artificial Sequence
  synthetic
atgctg

SEQ ID NO: 1310 - DNA, 6 bp, Artificial Sequence
  synthetic
atggac

SEQ ID NO: 1311 - DNA, 6 bp, Artificial Sequence
  synthetic
atggca

SEQ ID NO: 1312 - DNA, 6 bp, Artificial Sequence
  synthetic
atgttc

SEQ ID NO: 1313 - DNA, 6 bp, Artificial Sequence
  synthetic
attact

SEQ ID NO: 1314 - DNA, 6 bp, Artificial Sequence
  synthetic
attcac

SEQ ID NO: 1315 - DNA, 6 bp, Artificial Sequence
  synthetic
attcag

SEQ ID NO: 1316 - DNA, 6 bp, Artificial Sequence
  synthetic
attctg

SEQ ID NO: 1317 - DNA, 6 bp, Artificial Sequence
  synthetic
atttca

SEQ ID NO: 1318 - DNA, 6 bp, Artificial Sequence
  synthetic
caacgc

SEQ ID NO: 1319 - DNA, 6 bp, Artificial Sequence
  synthetic
caacgt

SEQ ID NO: 1320 - DNA, 6 bp, Artificial Sequence
  synthetic
caactg

SEQ ID NO: 1321 - DNA, 6 bp, Artificial Sequence
  synthetic
caaggc

SEQ ID NO: 1322 - DNA, 6 bp, Artificial Sequence
  synthetic
cacaac

SEQ ID NO: 1323 - DNA, 6 bp, Artificial Sequence
  synthetic
cacact

SEQ ID NO: 1324 - DNA, 6 bp, Artificial Sequence
  synthetic
caccat

SEQ ID NO: 1325 - DNA, 6 bp, Artificial Sequence
  synthetic
caccgt

SEQ ID NO: 1326 - DNA, 6 bp, Artificial Sequence
  synthetic
cacgct

SEQ ID NO: 1327 - DNA, 6 bp, Artificial Sequence
  synthetic
cactgc

SEQ ID NO: 1328 - DNA, 6 bp, Artificial Sequence
  synthetic
cacttc

SEQ ID NO: 1329 - DNA, 6 bp, Artificial Sequence
  synthetic
cagact

SEQ ID NO: 1330 - DNA, 6 bp, Artificial Sequence
  synthetic
cagaga

SEQ ID NO: 1331 - DNA, 6 bp, Artificial Sequence
  synthetic
caggct

SEQ ID NO: 1332 - DNA, 6 bp, Artificial Sequence
  synthetic
cagtgg

SEQ ID NO: 1333 - DNA, 6 bp, Artificial Sequence
  synthetic
cagtgt

SEQ ID NO: 1334 - DNA, 6 bp, Artificial Sequence
  synthetic
catcat

SEQ ID NO: 1335 - DNA, 6 bp, Artificial Sequence
  synthetic
cattga

SEQ ID NO: 1336 - DNA, 6 bp, Artificial Sequence
  synthetic
ccacaa

SEQ ID NO: 1337 - DNA, 6 bp, Artificial Sequence
  synthetic
ccagat

SEQ ID NO: 1338 - DNA, 6 bp, Artificial Sequence
  synthetic
ccatca

SEQ ID NO: 1339 - DNA, 6 bp, Artificial Sequence
  synthetic
cccatc

SEQ ID NO: 1340 - DNA, 6 bp, Artificial Sequence
  synthetic
cccctg

SEQ ID NO: 1341 - DNA, 6 bp, Artificial Sequence
  synthetic
cccgca

SEQ ID NO: 1342 - DNA, 6 bp, Artificial Sequence
  synthetic
ccgaca

SEQ ID NO: 1343 - DNA, 6 bp, Artificial Sequence
  synthetic
ccgttt

SEQ ID NO: 1344 - DNA, 6 bp, Artificial Sequence
  synthetic
cctaat

SEQ ID NO: 1345 - DNA, 6 bp, Artificial Sequence
  synthetic
cctaga

SEQ ID NO: 1346 - DNA, 6 bp, Artificial Sequence
  synthetic
cctgtg

SEQ ID NO: 1347 - DNA, 6 bp, Artificial Sequence
  synthetic
cgacat

SEQ ID NO: 1348 - DNA, 6 bp, Artificial Sequence
  synthetic
cgagtt

SEQ ID NO: 1349 - DNA, 6 bp, Artificial Sequence
  synthetic
cgcaac

SEQ ID NO: 1350 - DNA, 6 bp, Artificial Sequence
  synthetic
cgcaca

SEQ ID NO: 1351 - DNA, 6 bp, Artificial Sequence
  synthetic
cgcact

SEQ ID NO: 1352 - DNA, 6 bp, Artificial Sequence
  synthetic
cgctgt

SEQ ID NO: 1353 - DNA, 6 bp, Artificial Sequence
  synthetic
cgtcaa

SEQ ID NO: 1354 - DNA, 6 bp, Artificial Sequence
  synthetic
cgtgct

SEQ ID NO: 1355 - DNA, 6 bp, Artificial Sequence
  synthetic
cgtggt

SEQ ID NO: 1356 - DNA, 6 bp, Artificial Sequence
  synthetic
cgttac

SEQ ID NO: 1357 - DNA, 6 bp, Artificial Sequence
  synthetic
ctaggt

SEQ ID NO: 1358 - DNA, 6 bp, Artificial Sequence
  synthetic
ctcaca

SEQ ID NO: 1359 - DNA, 6 bp, Artificial Sequence
  synthetic
ctcatg

SEQ ID NO: 1360 - DNA, 6 bp, Artificial Sequence
  synthetic
ctcctg

SEQ ID NO: 1361 - DNA, 6 bp, Artificial Sequence
  synthetic
ctcctt

SEQ ID NO: 1362 - DNA, 6 bp, Artificial Sequence
  synthetic
ctctgc

SEQ ID NO: 1363 - DNA, 6 bp, Artificial Sequence
  synthetic
ctgagc

SEQ ID NO: 1364 - DNA, 6 bp, Artificial Sequence
  synthetic
ctgata

SEQ ID NO: 1365 - DNA, 6 bp, Artificial Sequence
  synthetic
ctgcaa

SEQ ID NO: 1366 - DNA, 6 bp, Artificial Sequence
  synthetic
ctgcct

SEQ ID NO: 1367 - DNA, 6 bp, Artificial Sequence
  synthetic
ctgcta

SEQ ID NO: 1368 - DNA, 6 bp, Artificial Sequence
  synthetic
ctgctg

SEQ ID NO: 1369 - DNA, 6 bp, Artificial Sequence
  synthetic
ctggtc

SEQ ID NO: 1370 - DNA, 6 bp, Artificial Sequence
  synthetic
ctgtgt

SEQ ID NO: 1371 - DNA, 6 bp, Artificial Sequence
  synthetic
cttgag

SEQ ID NO: 1372 - DNA, 6 bp, Artificial Sequence
  synthetic
cttgca

SEQ ID NO: 1373 - DNA, 6 bp, Artificial Sequence
  synthetic
ctttat

SEQ ID NO: 1374 - DNA, 6 bp, Artificial Sequence
  synthetic
ctttca

SEQ ID NO: 1375 - DNA, 6 bp, Artificial Sequence
  synthetic
gacacc

SEQ ID NO: 1376 - DNA, 6 bp, Artificial Sequence
  synthetic
gacata

SEQ ID NO: 1377 - DNA, 6 bp, Artificial Sequence
  synthetic
gaccta

SEQ ID NO: 1378 - DNA, 6 bp, Artificial Sequence
  synthetic
gacgcc

SEQ ID NO: 1379 - DNA, 6 bp, Artificial Sequence
  synthetic
gactcc

SEQ ID NO: 1380 - DNA, 6 bp, Artificial Sequence
  synthetic
gactgc

SEQ ID NO: 1381 - DNA, 6 bp, Artificial Sequence
  synthetic
gagatc

SEQ ID NO: 1382 - DNA, 6 bp, Artificial Sequence
  synthetic
gagcat

SEQ ID NO: 1383 - DNA, 6 bp, Artificial Sequence
  synthetic
gatagg

SEQ ID NO: 1384 - DNA, 6 bp, Artificial Sequence
  synthetic
gatatt

SEQ ID NO: 1385 - DNA, 6 bp, Artificial Sequence
  synthetic
gatgca

SEQ ID NO: 1386 - DNA, 6 bp, Artificial Sequence
  synthetic
gattct

SEQ ID NO: 1387 - DNA, 6 bp, Artificial Sequence
  synthetic
gcaacc

SEQ ID NO: 1388 - DNA, 6 bp, Artificial Sequence
  synthetic
gcaacg

SEQ ID NO: 1389 - DNA, 6 bp, Artificial Sequence
  synthetic
gcaact

SEQ ID NO: 1390 - DNA, 6 bp, Artificial Sequence
  synthetic
gcacaa

SEQ ID NO: 1391 - DNA, 6 bp, Artificial Sequence
  synthetic
gcacat

SEQ ID NO: 1392 - DNA, 6 bp, Artificial Sequence
  synthetic
gcacct

SEQ ID NO: 1393 - DNA, 6 bp, Artificial Sequence
  synthetic
gcactg

SEQ ID NO: 1394 - DNA, 6 bp, Artificial Sequence
  synthetic
gcatct

SEQ ID NO: 1395 - DNA, 6 bp, Artificial Sequence
  synthetic
gccata

SEQ ID NO: 1396 - DNA, 6 bp, Artificial Sequence
  synthetic
gctcac

SEQ ID NO: 1397 - DNA, 6 bp, Artificial Sequence
  synthetic
gctgcc

SEQ ID NO: 1398 - DNA, 6 bp, Artificial Sequence
  synthetic
gctgct

SEQ ID NO: 1399 - DNA, 6 bp, Artificial Sequence
  synthetic
gctgtt

SEQ ID NO: 1400 - DNA, 6 bp, Artificial Sequence
  synthetic
gcttcg

SEQ ID NO: 1401 - DNA, 6 bp, Artificial Sequence
  synthetic
gctttc

SEQ ID NO: 1402 - DNA, 6 bp, Artificial Sequence
  synthetic
ggaaat

SEQ ID NO: 1403 - DNA, 6 bp, Artificial Sequence
  synthetic
ggatat

SEQ ID NO: 1404 - DNA, 6 bp, Artificial Sequence
  synthetic
ggatgt

SEQ ID NO: 1405 - DNA, 6 bp, Artificial Sequence
  synthetic
ggcaac

SEQ ID NO: 1406 - DNA, 6 bp, Artificial Sequence
  synthetic
ggcaat

SEQ ID NO: 1407 - DNA, 6 bp, Artificial Sequence
  synthetic
ggcaca

SEQ ID NO: 1408 - DNA, 6 bp, Artificial Sequence
  synthetic
ggcact

SEQ ID NO: 1409 - DNA, 6 bp, Artificial Sequence
  synthetic
ggcaga

SEQ ID NO: 1410 - DNA, 6 bp, Artificial Sequence
  synthetic
ggccag

SEQ ID NO: 1411 - DNA, 6 bp, Artificial Sequence
  synthetic
ggcctg

SEQ ID NO: 1412 - DNA, 6 bp, Artificial Sequence
  synthetic
ggcctt

SEQ ID NO: 1413 - DNA, 6 bp, Artificial Sequence
  synthetic
ggcttc

SEQ ID NO: 1414 - DNA, 6 bp, Artificial Sequence
  synthetic
ggggta

SEQ ID NO: 1415 - DNA, 6 bp, Artificial Sequence
  synthetic
ggtatg

SEQ ID NO: 1416 - DNA, 6 bp, Artificial Sequence
  synthetic
ggtcta

SEQ ID NO: 1417 - DNA, 6 bp, Artificial Sequence
  synthetic
ggttat

SEQ ID NO: 1418 - DNA, 6 bp, Artificial Sequence
  synthetic
gtacca

SEQ ID NO: 1419 - DNA, 6 bp, Artificial Sequence
  synthetic
gtatca

SEQ ID NO: 1420 - DNA, 6 bp, Artificial Sequence
  synthetic
gtctac

SEQ ID NO: 1421 - DNA, 6 bp, Artificial Sequence
  synthetic
gtctga

SEQ ID NO: 1422 - DNA, 6 bp, Artificial Sequence
  synthetic
gtgaat

SEQ ID NO: 1423 - DNA, 6 bp, Artificial Sequence
  synthetic
gtgcta

SEQ ID NO: 1424 - DNA, 6 bp, Artificial Sequence
  synthetic
gtgctg

SEQ ID NO: 1425 - DNA, 6 bp, Artificial Sequence
  synthetic
gtggtc

SEQ ID NO: 1426 - DNA, 6 bp, Artificial Sequence
  synthetic
gttact

SEQ ID NO: 1427 - DNA, 6 bp, Artificial Sequence
  synthetic
gttatc

SEQ ID NO: 1428 - DNA, 6 bp, Artificial Sequence
  synthetic
gttttg

SEQ ID NO: 1429 - DNA, 6 bp, Artificial Sequence
  synthetic
taataa

SEQ ID NO: 1430 - DNA, 6 bp, Artificial Sequence
  synthetic
tactgc

SEQ ID NO: 1431 - DNA, 6 bp, Artificial Sequence
  synthetic
tagatt

SEQ ID NO: 1432 - DNA, 6 bp, Artificial Sequence
  synthetic
taggct

SEQ ID NO: 1433 - DNA, 6 bp, Artificial Sequence
  synthetic
tatggc

SEQ ID NO: 1434 - DNA, 6 bp, Artificial Sequence
  synthetic
tatggg

SEQ ID NO: 1435 - DNA, 6 bp, Artificial Sequence
  synthetic
tatttc

SEQ ID NO: 1436 - DNA, 6 bp, Artificial Sequence
  synthetic
tcacag

SEQ ID NO: 1437 - DNA, 6 bp, Artificial Sequence
  synthetic
tcacta

SEQ ID NO: 1438 - DNA, 6 bp, Artificial Sequence
  synthetic
tcagag

SEQ ID NO: 1439 - DNA, 6 bp, Artificial Sequence
  synthetic
tcaggc

SEQ ID NO: 1440 - DNA, 6 bp, Artificial Sequence
  synthetic
tcatgg

SEQ ID NO: 1441 - DNA, 6 bp, Artificial Sequence
  synthetic
tcattt

SEQ ID NO: 1442 - DNA, 6 bp, Artificial Sequence
  synthetic
tccaac

SEQ ID NO: 1443 - DNA, 6 bp, Artificial Sequence
  synthetic
tccaga

SEQ ID NO: 1444 - DNA, 6 bp, Artificial Sequence
  synthetic
tcctgt

SEQ ID NO: 1445 - DNA, 6 bp, Artificial Sequence
  synthetic
tccttg

SEQ ID NO: 1446 - DNA, 6 bp, Artificial Sequence
  synthetic
tcgacc

SEQ ID NO: 1447 - DNA, 6 bp, Artificial Sequence
  synthetic
tcggta

SEQ ID NO: 1448 - DNA, 6 bp, Artificial Sequence
  synthetic
tcggtg

SEQ ID NO: 1449 - DNA, 6 bp, Artificial Sequence
  synthetic
tctcag

SEQ ID NO: 1450 - DNA, 6 bp, Artificial Sequence
  synthetic
tctgct

SEQ ID NO: 1451 - DNA, 6 bp, Artificial Sequence
  synthetic
tctgtc

SEQ ID NO: 1452 - DNA, 6 bp, Artificial Sequence
  synthetic
tcttct

SEQ ID NO: 1453 - DNA, 6 bp, Artificial Sequence
  synthetic
tgaagt

SEQ ID NO: 1454 - DNA, 6 bp, Artificial Sequence
  synthetic
tgaata

SEQ ID NO: 1455 - DNA, 6 bp, Artificial Sequence
  synthetic
tgacat

SEQ ID NO: 1456 - DNA, 6 bp, Artificial Sequence
  synthetic
tgaccg

SEQ ID NO: 1457 - DNA, 6 bp, Artificial Sequence
  synthetic
tgactt

SEQ ID NO: 1458 - DNA, 6 bp, Artificial Sequence
  synthetic
tgagat

SEQ ID NO: 1459 - DNA, 6 bp, Artificial Sequence
  synthetic
tgagcg

SEQ ID NO: 1460 - DNA, 6 bp, Artificial Sequence
  synthetic
tgataa

SEQ ID NO: 1461 - DNA, 6 bp, Artificial Sequence
  synthetic
tgattc

SEQ ID NO: 1462 - DNA, 6 bp, Artificial Sequence
  synthetic
tgcacc

SEQ ID NO: 1463 - DNA, 6 bp, Artificial Sequence
  synthetic
tgcagg

SEQ ID NO: 1464 - DNA, 6 bp, Artificial Sequence
  synthetic
tgcatc

SEQ ID NO: 1465 - DNA, 6 bp, Artificial Sequence
  synthetic
tgccac

SEQ ID NO: 1466 - DNA, 6 bp, Artificial Sequence
  synthetic
tgccgt

SEQ ID NO: 1467 - DNA, 6 bp, Artificial Sequence
  synthetic
tgctag

SEQ ID NO: 1468 - DNA, 6 bp, Artificial Sequence
  synthetic
tgctga

SEQ ID NO: 1469 - DNA, 6 bp, Artificial Sequence
  synthetic
tgctgg

SEQ ID NO: 1470 - DNA, 6 bp, Artificial Sequence
  synthetic
tgctgt

SEQ ID NO: 1471 - DNA, 6 bp, Artificial Sequence
  synthetic
tggact

SEQ ID NO: 1472 - DNA, 6 bp, Artificial Sequence
  synthetic
tggagt

SEQ ID NO: 1473 - DNA, 6 bp, Artificial Sequence
  synthetic
tggcag

SEQ ID NO: 1474 - DNA, 6 bp, Artificial Sequence
  synthetic
tggcta

SEQ ID NO: 1475 - DNA, 6 bp, Artificial Sequence
  synthetic
tggtct

SEQ ID NO: 1476 - DNA, 6 bp, Artificial Sequence
  synthetic
tgtgac

SEQ ID NO: 1477 - DNA, 6 bp, Artificial Sequence
  synthetic
tgtgga

SEQ ID NO: 1478 - DNA, 6 bp, Artificial Sequence
  synthetic
tgtgtg

SEQ ID NO: 1479 - DNA, 6 bp, Artificial Sequence
  synthetic
tgttat

SEQ ID NO: 1480 - DNA, 6 bp, Artificial Sequence
  synthetic
tgtttc

SEQ ID NO: 1481 - DNA, 6 bp, Artificial Sequence
  synthetic
ttactg

SEQ ID NO: 1482 - DNA, 6 bp, Artificial Sequence
  synthetic
ttattt

SEQ ID NO: 1483 - DNA, 6 bp, Artificial Sequence
  synthetic
ttcagg

SEQ ID NO: 1484 - DNA, 6 bp, Artificial Sequence
  synthetic
ttcctg

SEQ ID NO: 1485 - DNA, 6 bp, Artificial Sequence
  synthetic
ttcgac

SEQ ID NO: 1486 - DNA, 6 bp, Artificial Sequence
  synthetic
ttcggc

SEQ ID NO: 1487 - DNA, 6 bp, Artificial Sequence
  synthetic
ttcttc

SEQ ID NO: 1488 - DNA, 6 bp, Artificial Sequence
  synthetic
ttgaat

SEQ ID NO: 1489 - DNA, 6 bp, Artificial Sequence
  synthetic
ttgaga

SEQ ID NO: 1490 - DNA, 6 bp, Artificial Sequence
  synthetic
ttgagt

SEQ ID NO: 1491 - DNA, 6 bp, Artificial Sequence
  synthetic
ttgcac

SEQ ID NO: 1492 - DNA, 6 bp, Artificial Sequence
  synthetic
ttggca

SEQ ID NO: 1493 - DNA, 6 bp, Artificial Sequence
  synthetic
ttgggc

SEQ ID NO: 1494 - DNA, 6 bp, Artificial Sequence
  synthetic
tttcaa

SEQ ID NO: 1495 - DNA, 6 bp, Artificial Sequence
  synthetic
tttcct

SEQ ID NO: 1496 - DNA, 6 bp, Artificial Sequence
  synthetic
tttgag

SEQ ID NO: 1497 - DNA, 6 bp, Artificial Sequence
  synthetic
tttgct

SEQ ID NO: 1498 - DNA, 6 bp, Artificial Sequence
  synthetic
tttggc

SEQ ID NO: 1499 - DNA, 10 bp, Artificial Sequence
  synthetic
tccgatctct

SEQ ID NO: 1500 - DNA, 10 bp, Artificial Sequence
  synthetic
tccgatctga

SEQ ID NO: 1501 - DNA, 11 bp, Artificial Sequence
  synthetic
ntccgatctct

SEQ ID NO: 1502 - DNA, 11 bp, Artificial Sequence
  synthetic
ntccgatctga

SEQ ID NO: 1503 - DNA, 27 bp, Artificial Sequence
  synthetic
ccgaactacccacttgcattnnnnnnn

SEQ ID NO: 1504 - DNA, 21 bp, Artificial Sequence
  synthetic
ccgaactacccacttgcattn

SEQ ID NO: 1505 - DNA, 29 bp, Artificial Sequence
  synthetic
ccactccatttgttcgtgtgnnnnnnnnn

SEQ ID NO: 1506 - DNA, 20 bp, Artificial Sequence
  synthetic
ccactccatttgttcgtgtg

SEQ ID NO: 1507 - DNA, 20 bp, Artificial Sequence
  synthetic
ccgaactacccacttgcatt

SEQ ID NO: 1508 - DNA, 26 bp, Artificial Sequence
  synthetic
aattaatacgactcactatagggaga

SEQ ID NO: 1509 - DNA, 23 bp, Artificial Sequence
  synthetic
atttaggtgacactatagaagng

SEQ ID NO: 1510 - DNA, 23 bp, Artificial Sequence
  synthetic
aattaaccctcactaaagggaga

SEQ ID NO: 1511 - DNA, 16 bp, Artificial Sequence
  synthetic
ggttcgccccgagaga

SEQ ID NO: 1512 - DNA, 14 bp, Artificial Sequence
  synthetic
ggacgccgccggaa

SEQ ID NO: 1513 - DNA, 16 bp, Artificial Sequence
  synthetic
ccgcgacgctttccaa

SEQ ID NO: 1514 - DNA, 21 bp, Artificial Sequence
  synthetic
gtagccaaatgcctcgtcatc

SEQ ID NO: 1515 - DNA, 24 bp, Artificial Sequence
  synthetic
cagtgggaatctcgttcatccatt

SEQ ID NO: 1516 - DNA, 17 bp, Artificial Sequence
  synthetic
atgcgcgtcactaatta

SEQ ID NO: 1517 - DNA, 25 bp, Artificial Sequence
  synthetic
ccgaaacgatctcaacctattctca

SEQ ID NO: 1518 - DNA, 15 bp, Artificial Sequence
  synthetic
gctccacgccagcga

SEQ ID NO: 1519 - DNA, 15 bp, Artificial Sequence
  synthetic
ccgggcttcttaccc

SEQ ID NO: 1520 - DNA, 23 bp, Artificial Sequence
  synthetic
gcgggtggtaaactccatctaag

SEQ ID NO: 1521 - DNA, 25 bp, Artificial Sequence
  synthetic
cccttacggtacttgttgactatcg

SEQ ID NO: 1522 - DNA, 16 bp, Artificial Sequence
  synthetic
tcgtgccggtatttag

SEQ ID NO: 1523 - DNA, 17 bp, Artificial Sequence
  synthetic
ggtgaccacgggtgacg

SEQ ID NO: 1524 - DNA, 21 bp, Artificial Sequence
  synthetic
ggatgtggtagccgtttctca

SEQ ID NO: 1525 - DNA, 16 bp, Artificial Sequence
  synthetic
tccctctccggaatcg

SEQ ID NO: 1526 - DNA, 28 bp, Artificial Sequence
  synthetic
accaagcataatatagcaaggactaacc

SEQ ID NO: 1527 - DNA, 25 bp, Artificial Sequence
  synthetic
tggctctccttgcaaagttatttct

SEQ ID NO: 1528 - DNA, 20 bp, Artificial Sequence
  synthetic
ccttctgcataatgaattaa

SEQ ID NO: 1529 - DNA, 19 bp, Artificial Sequence
  synthetic
gacaagcatcaagcacgca

SEQ ID NO: 1530 - DNA, 26 bp, Artificial Sequence
  synthetic
ctaaaggttaatcactgctgtttccc

SEQ ID NO: 1531 - DNA, 17 bp, Artificial Sequence
  synthetic
caatgcagctcaaaacg

SEQ ID NO: 1532 - DNA, 25 bp, Artificial Sequence
  synthetic
gtcgaaggtggatttagcagtaaac

SEQ ID NO: 1533 - DNA, 17 bp, Artificial Sequence
  synthetic
tgtacgcgcttcagggc

SEQ ID NO: 1534 - DNA, 21 bp, Artificial Sequence
  synthetic
cctgttcaactaagcactcta

SEQ ID NO: 1535 - DNA, 20 bp, Artificial Sequence
  synthetic
aagcgttcaagctcaacacc

SEQ ID NO: 1536 - DNA, 20 bp, Artificial Sequence
  synthetic
ggtccaattgggtatgagga

SEQ ID NO: 1537 - DNA, 20 bp, Artificial Sequence
  synthetic
gcataagcctgcgtcagatt

SEQ ID NO: 1538 - DNA, 24 bp, Artificial Sequence
  synthetic
ggttgattgtagatattgggctgt

SEQ ID NO: 1539 - DNA, 20 bp, Artificial Sequence
  synthetic
tacctgaccgctgagatcct

SEQ ID NO: 1540 - DNA, 20 bp, Artificial Sequence
  synthetic
agcttgttgagctcctcgtc

SEQ ID NO: 1541 - DNA, 20 bp, Artificial Sequence
  synthetic
gacatctgtcaccccattga

SEQ ID NO: 1542 - DNA, 20 bp, Artificial Sequence
  synthetic
ctcctctatcggggatggtc

SEQ ID NO: 1543 - DNA, 20 bp, Artificial Sequence
  synthetic
ggagttctgggctgtagtgc

SEQ ID NO: 1544 - DNA, 20 bp, Artificial Sequence
  synthetic
gttttgacctgctccgtttc

SEQ ID NO: 1545 - DNA, 20 bp, Artificial Sequence
  synthetic
gctaagaggcgggaggatag

SEQ ID NO: 1546 - DNA, 20 bp, Artificial Sequence
  synthetic
ggttgttgctttgagggaag

SEQ ID NO: 1547 - DNA, 20 bp, Artificial Sequence
  synthetic
gctggtccgaaggtagtgag

SEQ ID NO: 1548 - DNA, 20 bp, Artificial Sequence
  synthetic
atgccaggagagtggaaact

SEQ ID NO: 1549 - DNA, 20 bp, Artificial Sequence
  synthetic
tccgagtgcagtggtgttta

SEQ ID NO: 1550 - DNA, 20 bp, Artificial Sequence
  synthetic
gtgggagtggagaaggaaca

SEQ ID NO: 1551 - DNA, 20 bp, Artificial Sequence
  synthetic
ggtccgatggtagtgggtta

SEQ ID NO: 1552 - DNA, 22 bp, Artificial Sequence
  synthetic
aaaaagccagtcaaatttagca

SEQ ID NO: 1553 - DNA, 20 bp, Artificial Sequence
  synthetic
tggcagtatcgtagccaatg

SEQ ID NO: 1554 - DNA, 20 bp, Artificial Sequence
  synthetic
ctgtcaaaaattgccaatgc

SEQ ID NO: 1555 - DNA, 20 bp, Artificial Sequence
  synthetic
cgcttcggcagcacatatac

SEQ ID NO: 1556 - DNA, 21 bp, Artificial Sequence
  synthetic
aaaatatggaacgcttcacga

SEQ ID NO: 1557 - RNA, 14 nt, Artificial Sequence
  Synthetic
gacggaugcggucu

SEQ ID NO: 1558 - DNA, 14 bp, Artificial Sequence
  RNA/DNA Hybrid Synthetic
gacggaugcggtgt

SEQ ID NO: 1559 - DNA, 53 bp, Artificial Sequence
  Synthetic
atgatacggcgaccaccgacactctttccctacacgacgctcttccgatctct

SEQ ID NO: 1560 - DNA, 36 bp, Artificial Sequence
  Synthetic
caagcagaagacggcatacgagctcttccgatctga